(19) United States
(12) Patent Application Publication
(10) Pub. No.: US 2025/0113725 A1
(43) Pub. Date: Apr. 3, 2025

(54) PLATINUM COMPLEX AND ORGANIC PHOTOELECTRONIC DEVICE COMPRISING THE SAME

(71) Applicant: GUANGDONG AGLAIA OPTOELECTRONIC MATERIALS CO., LTD, Foshan (CN)

(72) Inventors: Xinwei Wu, Foshan (CN); Huiyang Li, Foshan (CN); Lei Dai, Foshan (CN); Lifei Cai, Foshan (CN)

(21) Appl. No.: 18/714,904

(22) PCT Filed: Dec. 10, 2022
(86) PCT No.: PCT/CN2022/138233

§ 371 Date: May 30, 2024

(30) Foreign Application Priority Data
Dec. 14, 2021 (CN) 202111523121.0
Jun. 15, 2022 (CN) 202210671687.6

(51) Int. Cl. H10K 85/30; C07F 15/00; C09K 11/06; H10K 50/11; H10K 50/16

(52) U.S. Cl. CPC H10K 85/346; C07F 15/0086; C09K 11/06; C09K 2211/1007; C09K 2211/1011; C09K 2211/1014; C09K 2211/1029; C09K 2211/1044; C09K 2211/185; H10K 50/11; H10K 50/16

(57) ABSTRACT

The present application relates to a platinum complex and a device comprising the same. The platinum complex is a compound of a structure of a chemical formula (I). The device is an organic light-emitting diode, an organic thin film transistor, an organic photovoltaic device, a luminescent electrochemical cell, or a chemical sensor.

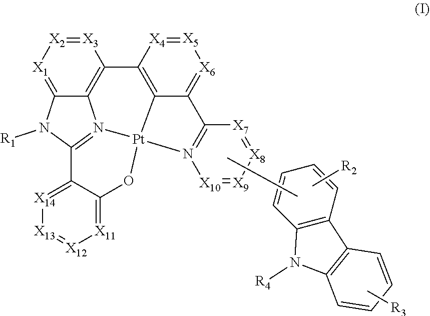

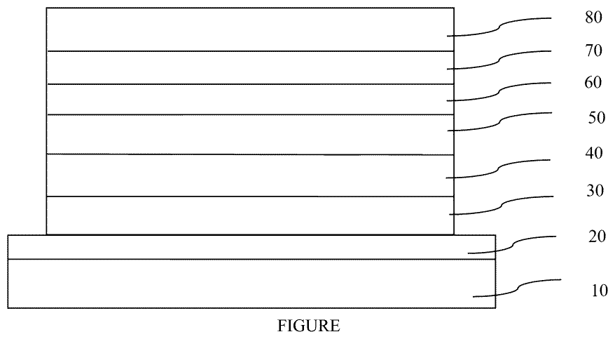

### CROSS-REFERENCE TO RELATED APPLICATIONS

This application is an U.S. national phase application under 35 U.S.C. § 371 based upon international patent application No. PCT/CN2022/138233 filed on Dec. 10, 2022, which itself claims priorities to Chinese patent application No. 202111523121.0 filed on Dec. 14, 2021 and Chinese patent application No. 202210671687.6 filed on Jun. 15, 2022. The contents of the above-identified applications are hereby incorporated herein in their entireties by reference.

### TECHNICAL FIELD

The present application relates to the field of luminescent materials, in particular to a carbazole-modified ONCN tetradentate ligand-containing platinum complex luminescent material and an application thereof.

### BACKGROUND

The organic light-emitting diode (OLED) was pioneered by Chinese-American professor Ching W. Tang in 1979 in the laboratory, and has received extensive attention and research due to its advantages of self-luminous, wide viewing angle, nearly infinite contrast, low power consumption, high response speed, potential for flexible and foldable applications, etc. In the field of OLED materials, the development of the doping material for the phosphorescent OLED light-emitting layer, primarily based on heavy metal organic complexes, has progressed rapidly and matured. The phosphorescent material can efficiently utilize the energy of the singlet and triplet excitons in the light-emitting process, with a theoretical internal quantum efficiency of 100%, and is a widely used luminescent material in the industry at present. The metal complex luminescent material has been applied in the industry, among which the metal iridium complex is a main conventional and industrialized doping material for the phosphorescent OLED light-emitting layer. The metal platinum offers a natural price advantage over iridium, and the complex thereof has also been greatly developed in recent years due to its excellent material stability resulted from its planarity. In recent years, the metal platinum complex has shown properties comparable to the iridium complex, but further improvements in luminous efficiency, service life, and the like are still required. There is an urgent need to have a luminescent material with a higher luminous efficiency and a longer service life in the industry. The ONCN tetradentate ligand-containing platinum complex molecule is simple in synthetic procedure, has multiple modification sites, and has a large space for modification. In addition, carbazole, one of the most commonly used efficient conjugate modification groups in the OLED material, can effectively address the concern in the material efficiency.

### SUMMARY

In view of the above-mentioned problems in the prior art, the present application provides a carbazole-modified ONCN tetradentate ligand-based platinum complex luminescent material. The material contains a fused aza-ring that accelerates the triplet radiation transition by changing the spin density distribution. The carbazole modification inhibits the molecular aggregation and reduces the intermolecular π-π effect in the material, thereby enhancing the thermal stability of the molecules and improving the luminous efficiency of the molecules.

The present application further provides an organic light-emitting diode including the platinum complex luminescent material. The platinum complex exhibits good photoelectric property and device lifespan when applied to the organic light-emitting diode.

The carbazole-modified ONCN tetradentate ligand-based platinum complex is a compound having a structure of formula (I):

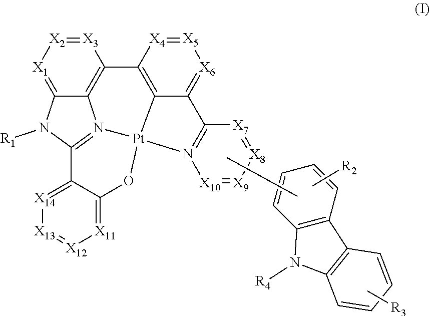

wherein,
X₁ to X₁₄ are each independently selected from the group consisting of N, C, and CR₀;
R₀ to R₄ are each independently selected from the group consisting of hydrogen, deuterium, halogen, amino, carbonyl, carboxyl, thioalkyl, cyano, sulfonyl, phosphino, a substituted or unsubstituted alkyl having 1 to 20 carbon atoms, a substituted or unsubstituted cycloalkyl having 3 to 20 ring carbon atoms, a substituted or unsubstituted alkenyl having 2 to 20 carbon atoms, a substituted or unsubstituted alkoxy having 1 to 20 carbon atoms, a substituted or unsubstituted aryl having 6 to 30 carbon atoms, and a substituted or unsubstituted heteroaryl having 3 to 30 carbon atoms;

adjacent groups of R₀ to R₄ are optionally linked to form a ring;

the “substituted” refers to substitution with halogen, amino, cyano, or C₁-C₄ alkyl;

a heteroatom in the heteroaryl includes one or more of N, S, or O.

In an embodiment, R₀ to R₄ are each independently selected from the group consisting of hydrogen, deuterium, halogen, amino, thioalkyl, cyano, a substituted or unsubstituted alkyl having 1 to 6 carbon atoms, a substituted or unsubstituted cycloalkyl having 3 to 6 ring carbon atoms, a substituted or unsubstituted alkenyl having 2 to 6 carbon atoms, a substituted or unsubstituted alkoxy having 1 to 6 carbon atoms, a substituted or unsubstituted aryl having 6 to 12 carbon atoms, and a substituted or unsubstituted heteroaryl having 3 to 6 carbon atoms.

In an embodiment, R₀, R₂, and R₃ are each independently selected from the group consisting of hydrogen, deuterium, halogen, a C₁-C₄ alkyl, cyano, a substituted or unsubstituted cycloalkyl having 3 to 6 ring carbon atoms, a substituted or unsubstituted aryl having 6 to 12 carbon atoms, and a substituted or unsubstituted heteroaryl having 3 to 6 carbon atoms.

In an embodiment, R₁ and R₄ are each independently selected from the group consisting of a substituted or unsubstituted cycloalkyl having 3 to 6 ring carbon atoms, a substituted or unsubstituted aryl having 6 to 12 carbon atoms, and a substituted or unsubstituted heteroaryl having 3 to 6 carbon atoms.

In an embodiment, R₀, R₂, and R₃ are each independently selected from the group consisting of hydrogen, deuterium, methyl, isopropyl, isobutyl, tert-butyl, cyano, substituted or unsubstituted cyclopentyl, substituted or unsubstituted cyclohexyl, substituted or unsubstituted phenyl, substituted or unsubstituted biphenyl, substituted or unsubstituted naphthyl, substituted or unsubstituted pyridinyl, substituted or unsubstituted pyrazinyl, and substituted or unsubstituted pyrimidinyl.

In an embodiment, R₁ and R₄ are each independently selected from the group consisting of substituted or unsubstituted cyclopentyl, substituted or unsubstituted cyclohexyl, substituted or unsubstituted phenyl, substituted or unsubstituted biphenyl, substituted or unsubstituted pyridinyl, substituted or unsubstituted pyrazinyl, substituted or unsubstituted pyrimidinyl, substituted or unsubstituted naphthyl, and substituted or unsubstituted quinolyl.

In an embodiment, X₁ to X₇ and X₉ to X₁₀ are each independently CR₀, X₈ is C, X₁₁ to X₁₄ are each independently CR₀ or N, and only one of X₁₁ to X₁₄ is N.

R₀ is selected from the group consisting of hydrogen, deuterium, methyl, isopropyl, isobutyl, tert-butyl, substituted or unsubstituted phenyl, substituted or unsubstituted biphenyl, and substituted or unsubstituted naphthyl. R₂ and R₃ are each independently selected from the group consisting of hydrogen, deuterium, methyl, isopropyl, isobutyl, and tert-butyl. R₁ and R₄ are each independently selected from the group consisting of substituted or unsubstituted phenyl, substituted or unsubstituted biphenyl, and substituted or unsubstituted naphthyl.

Examples of the platinum complex according to the present application are listed below. However, the platinum complex is not limited to the listed structures:

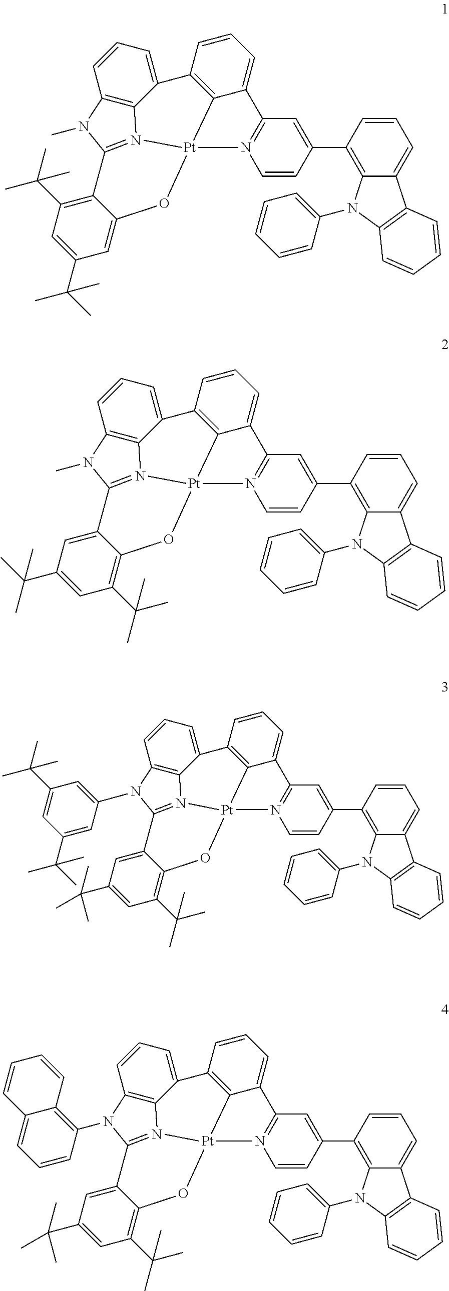

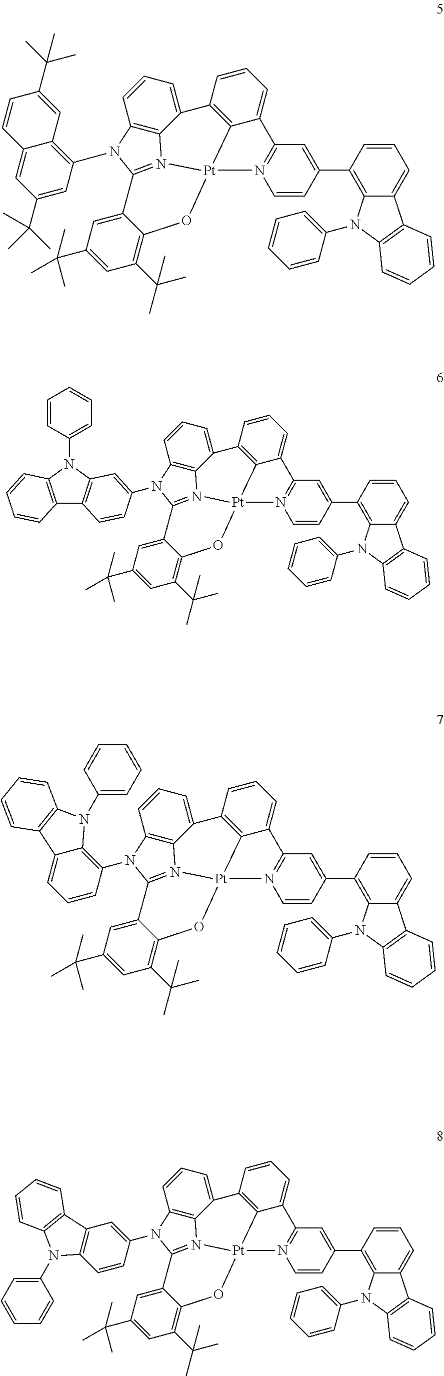

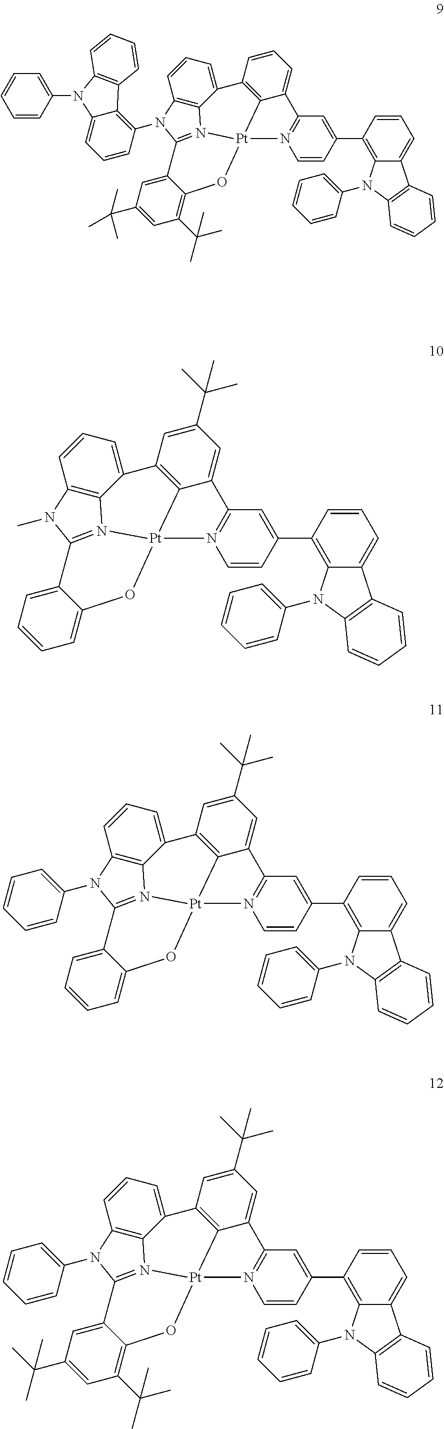

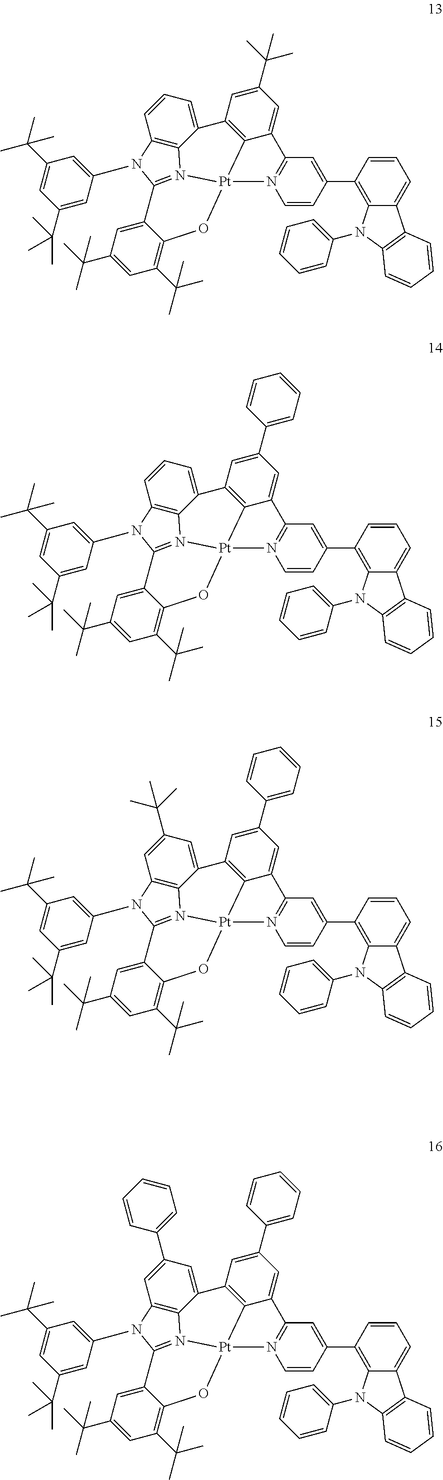

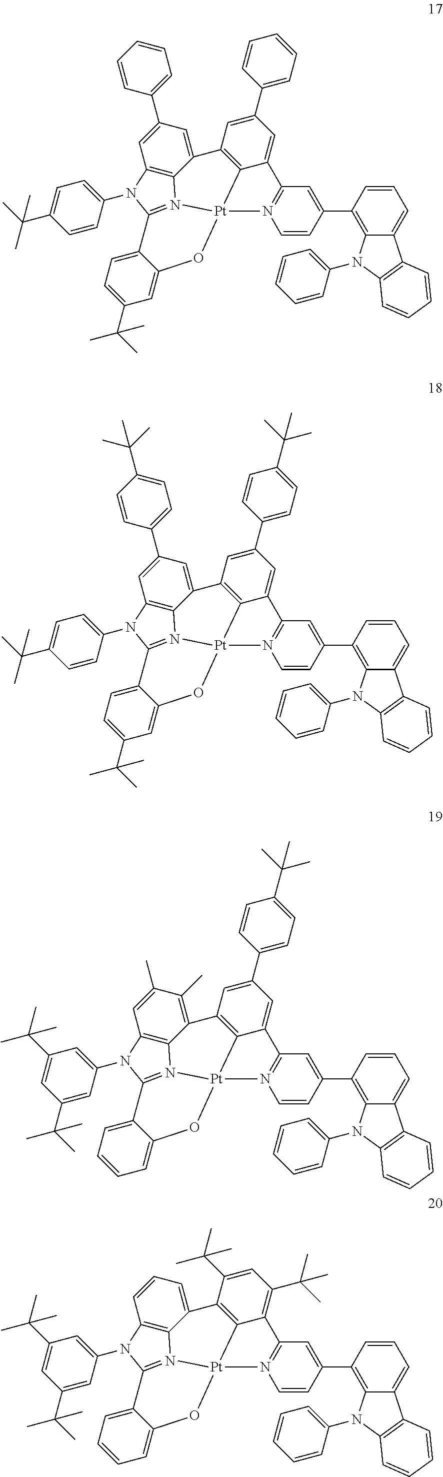

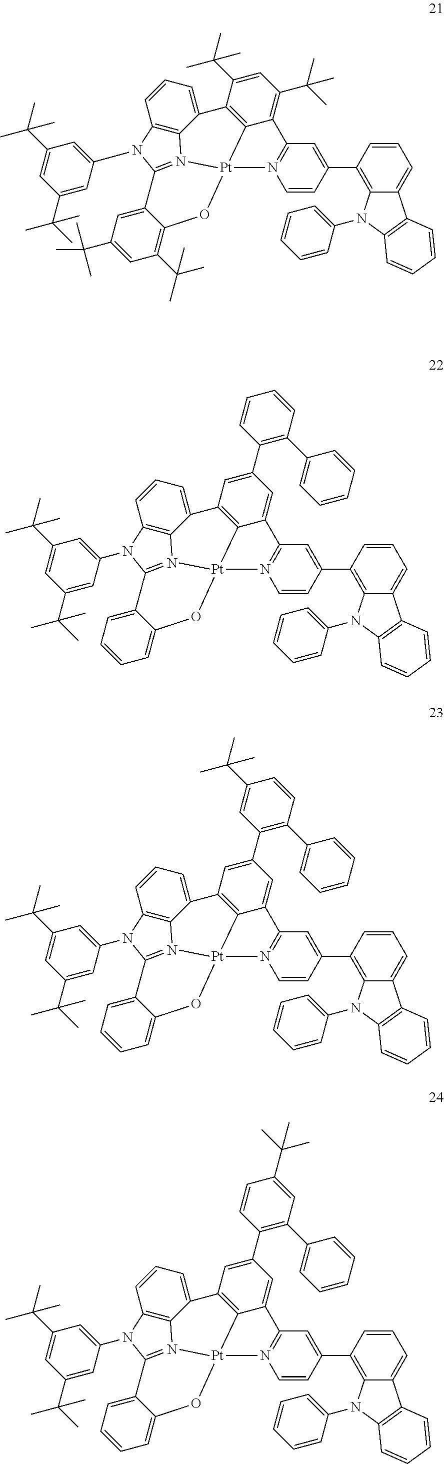

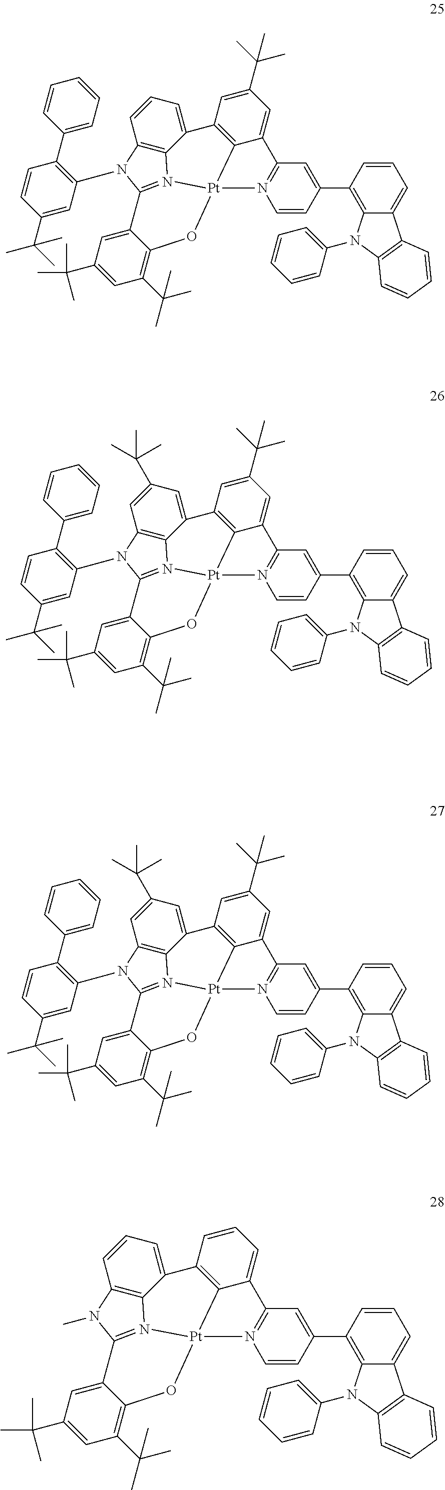

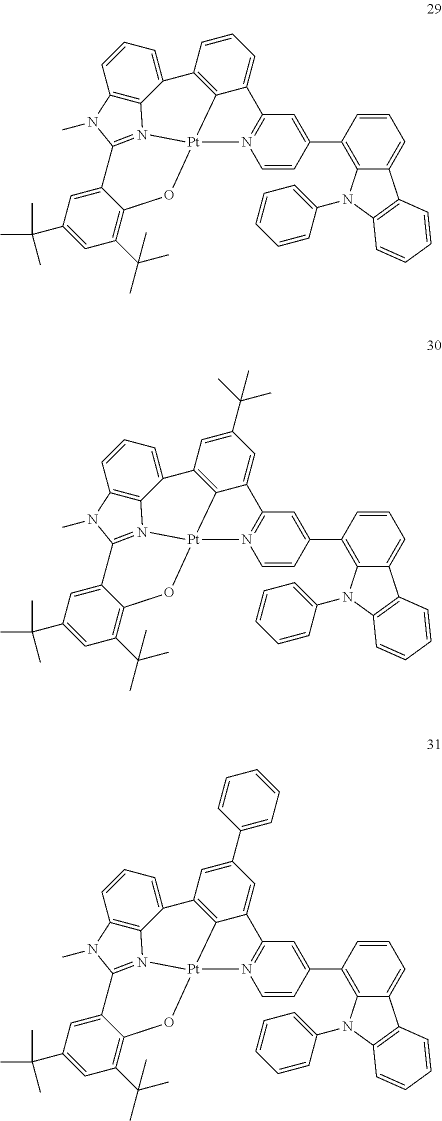

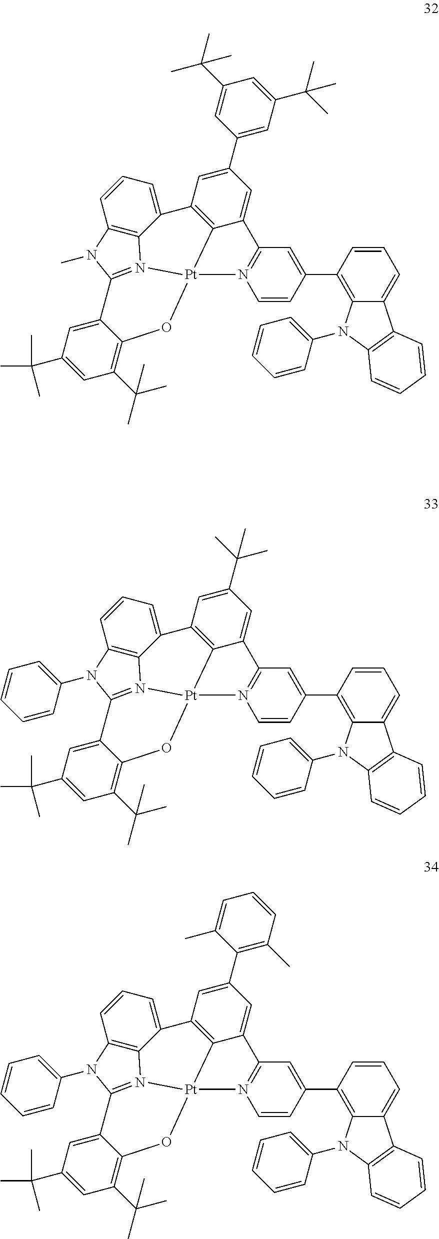

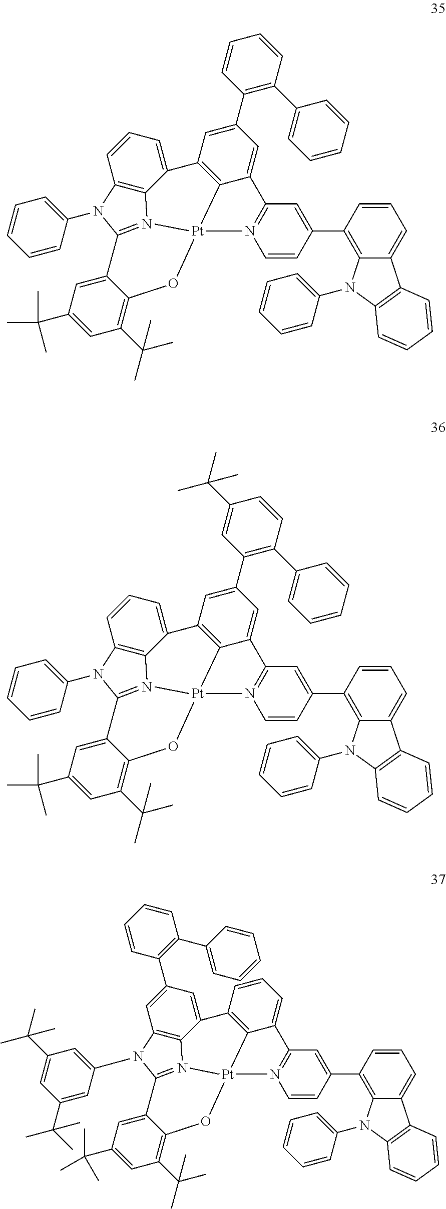

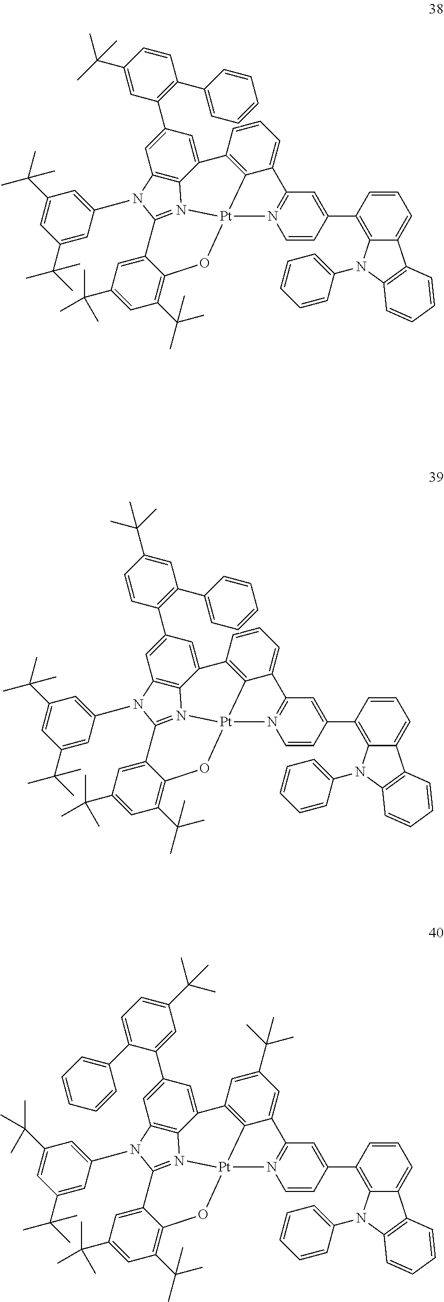

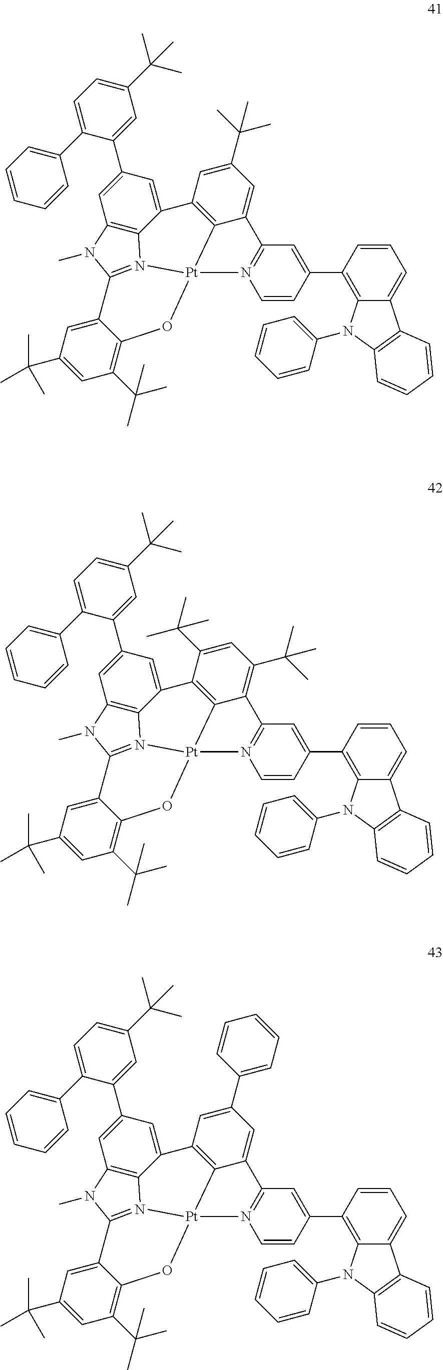

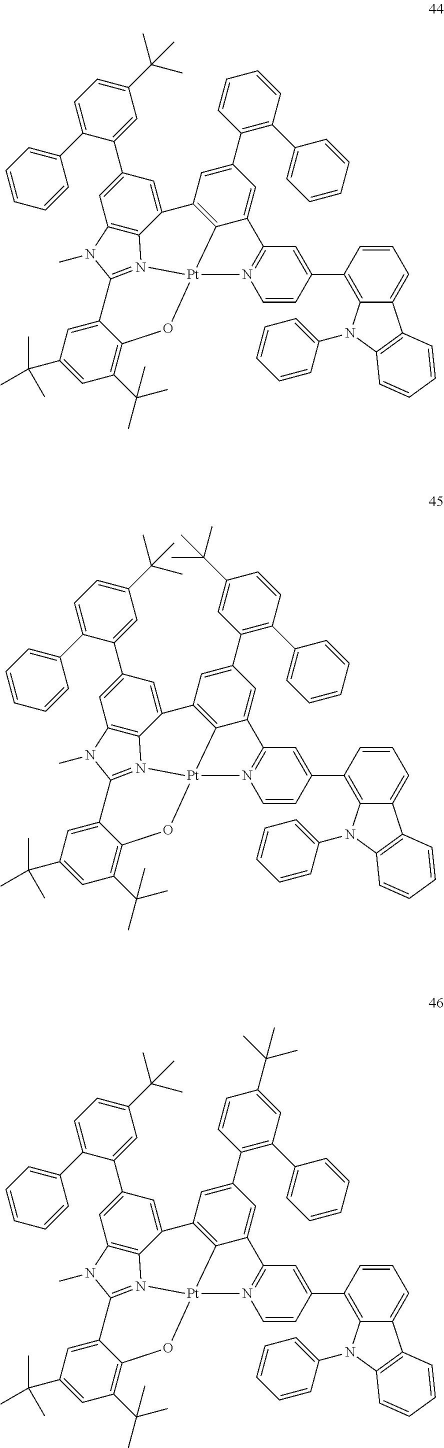

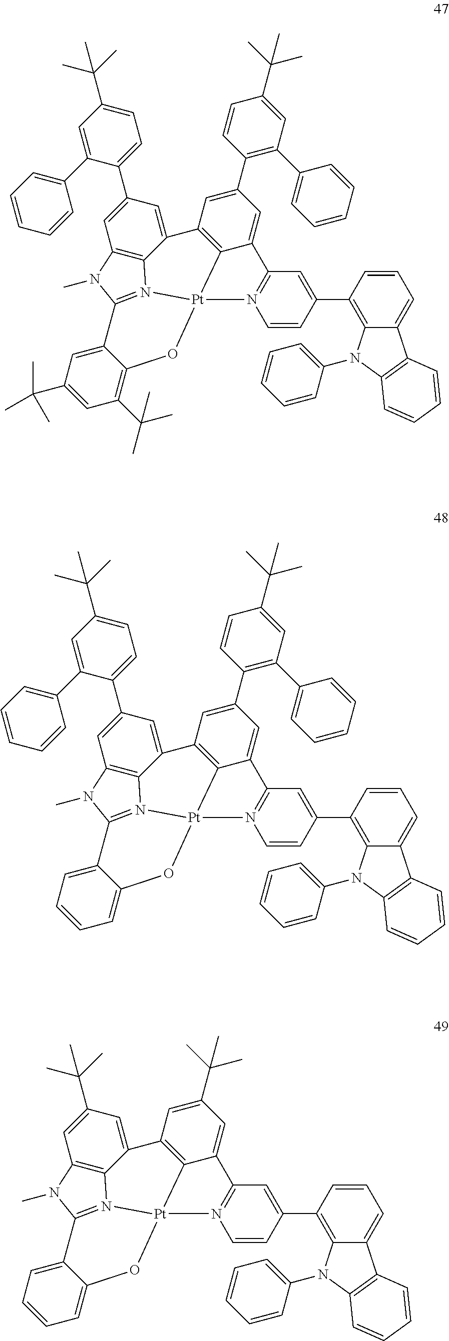

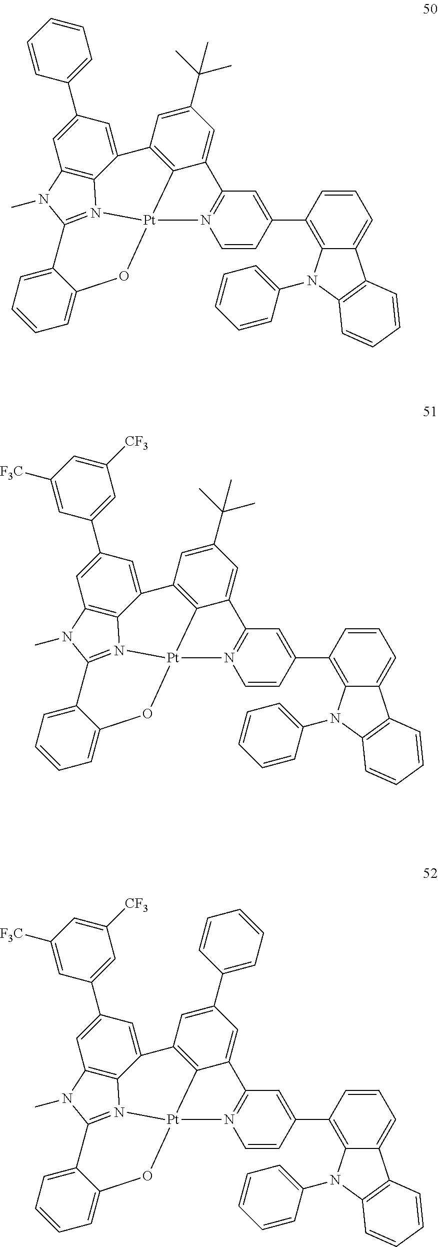

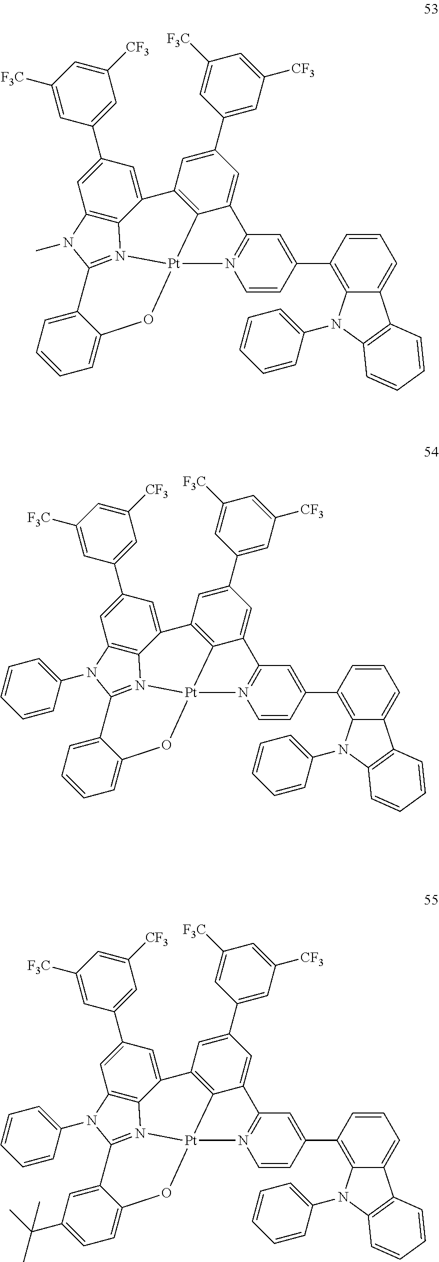

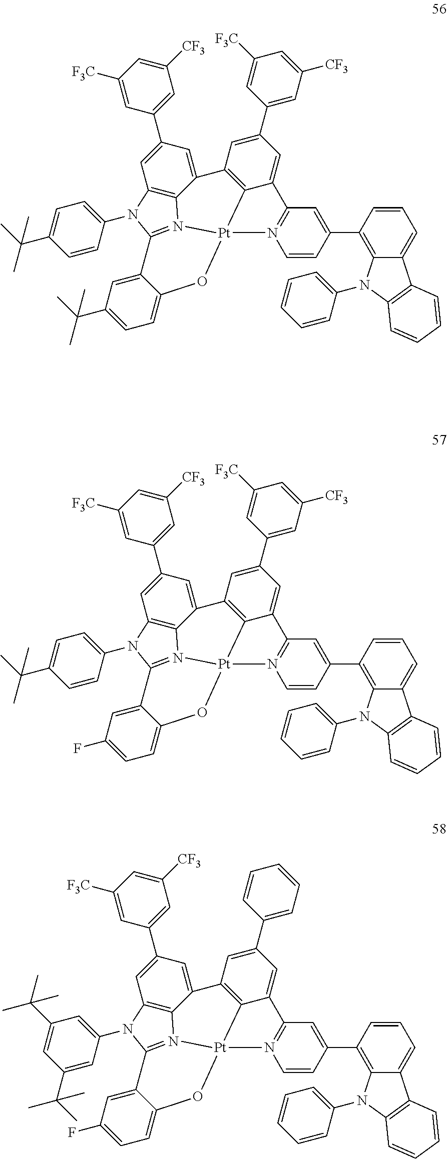

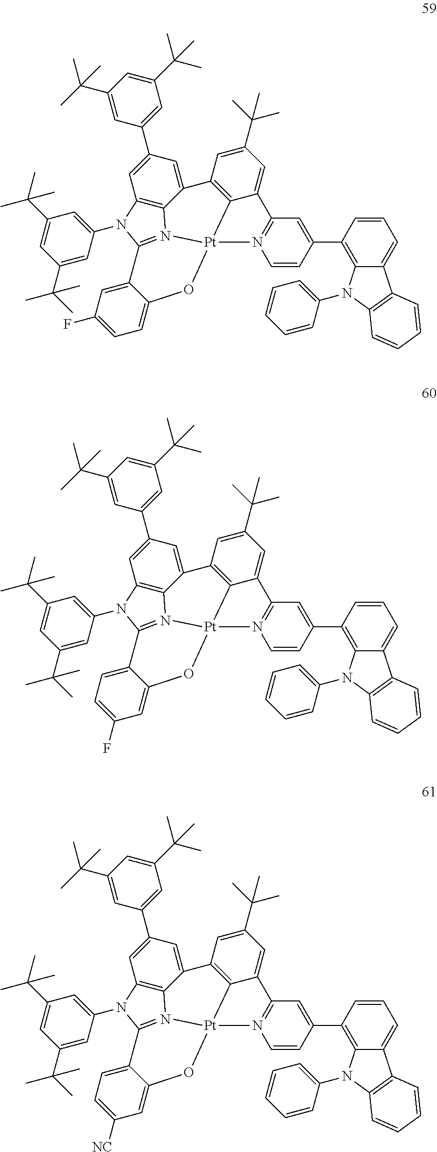

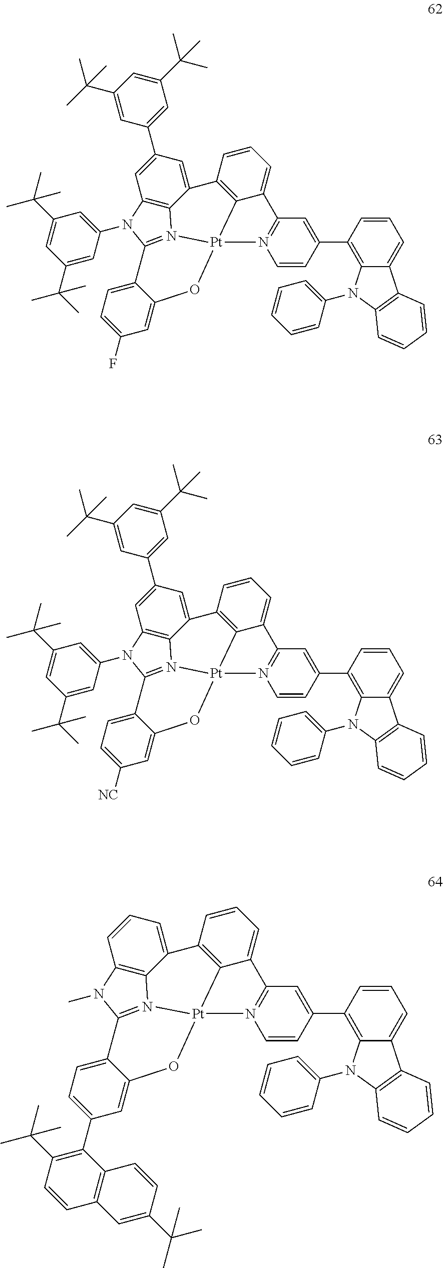

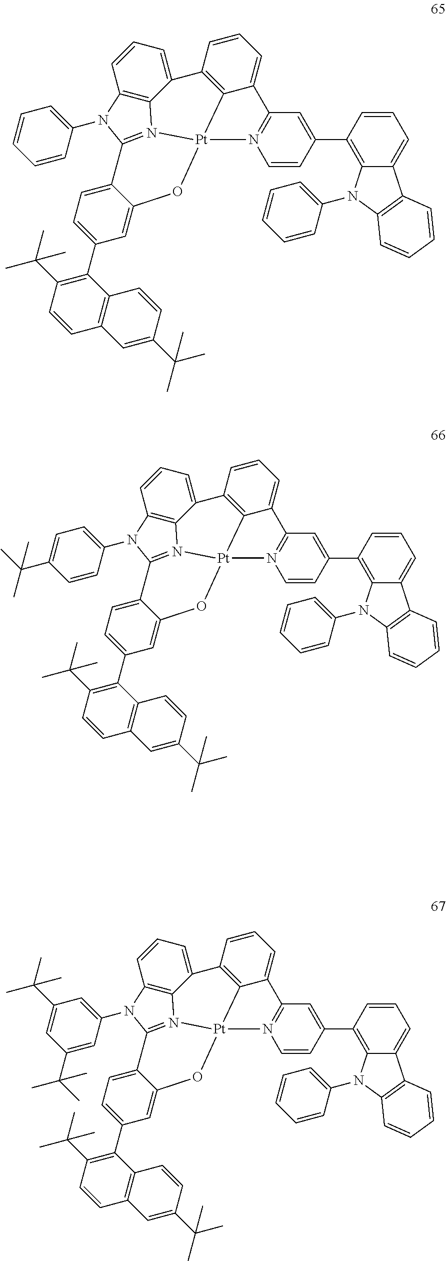

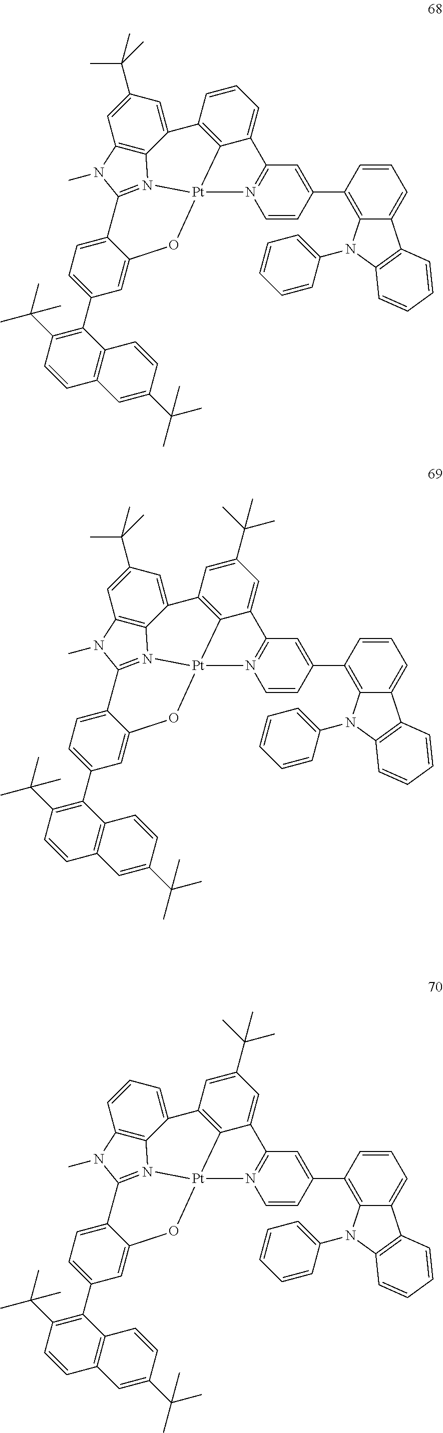

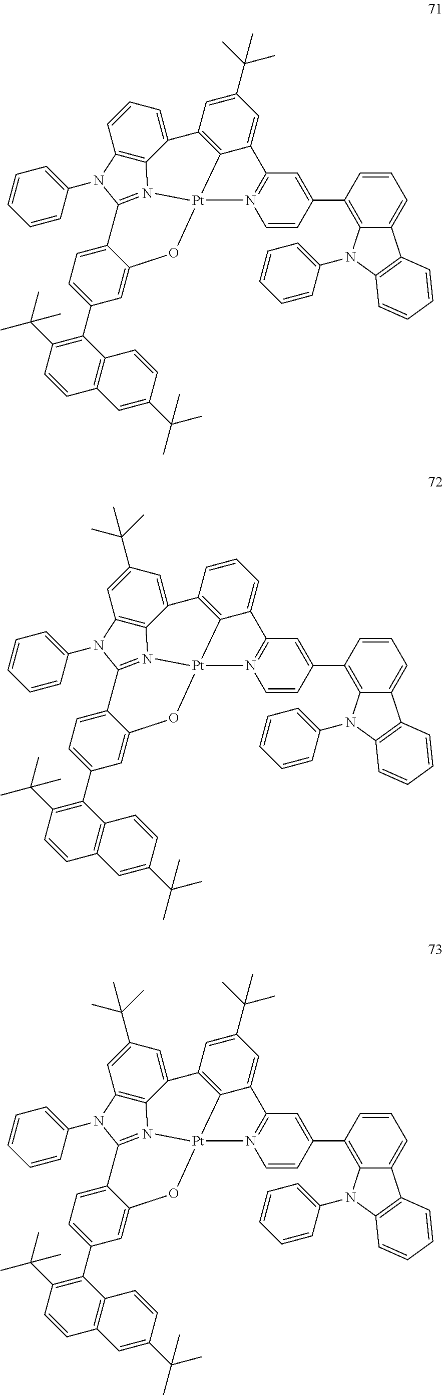

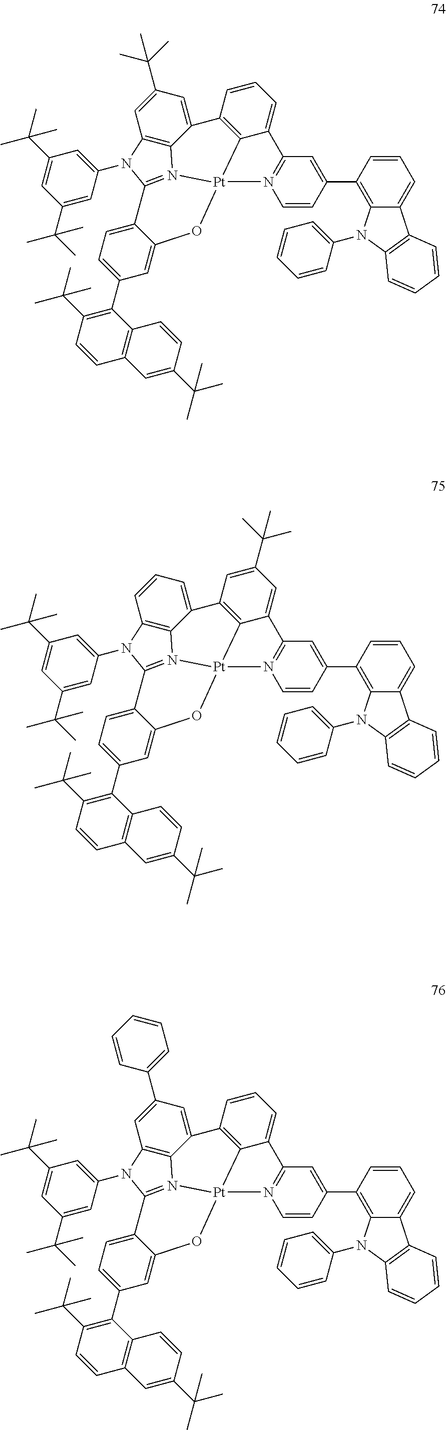

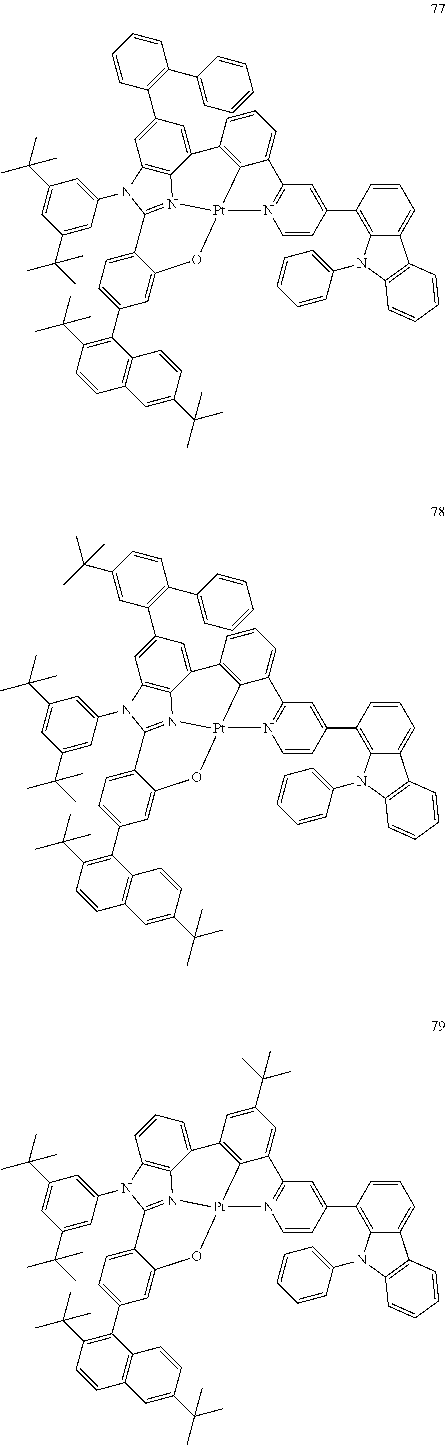

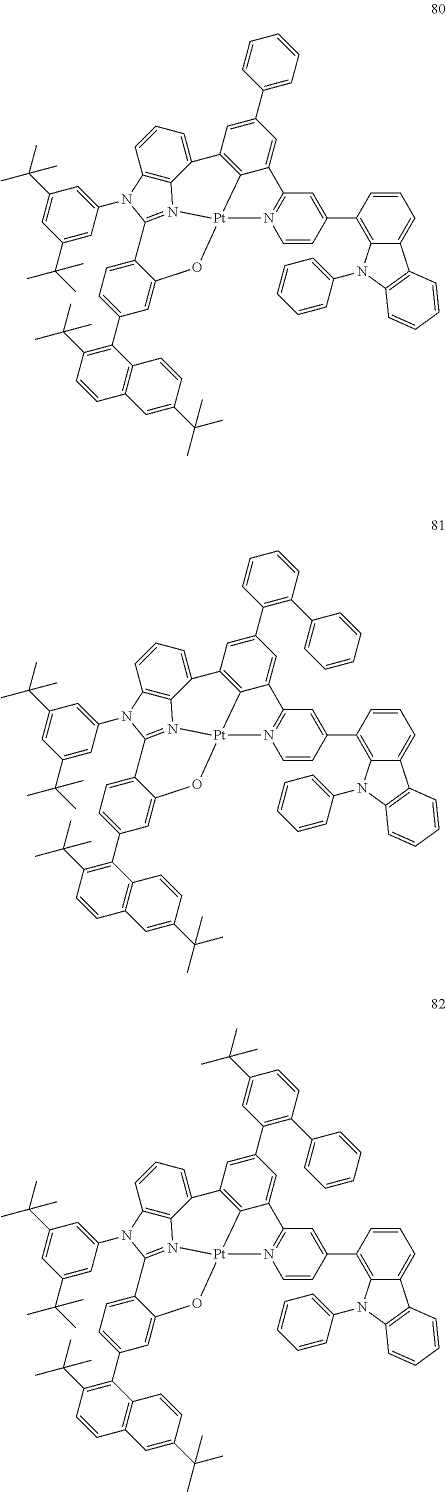

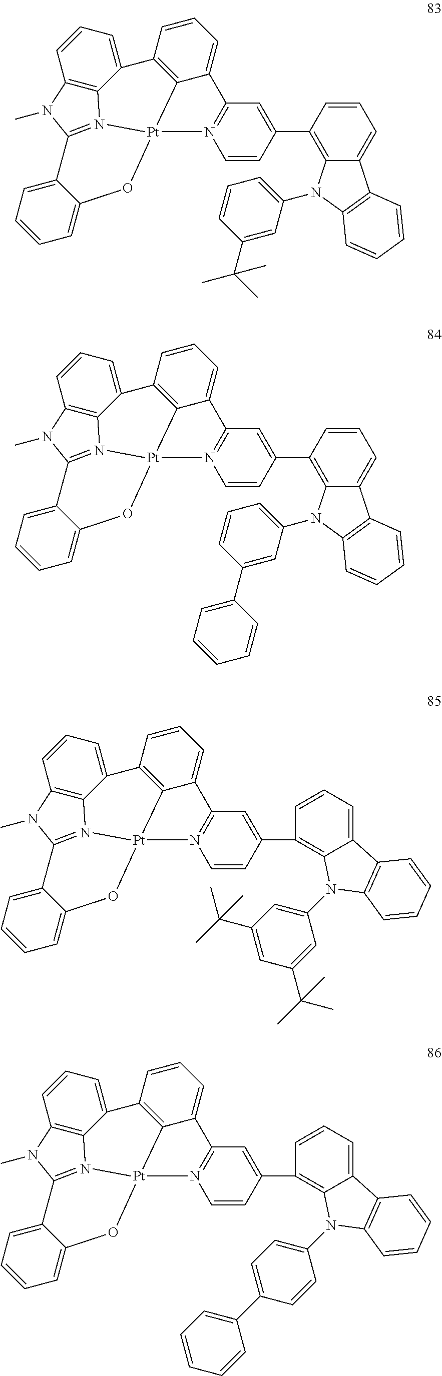

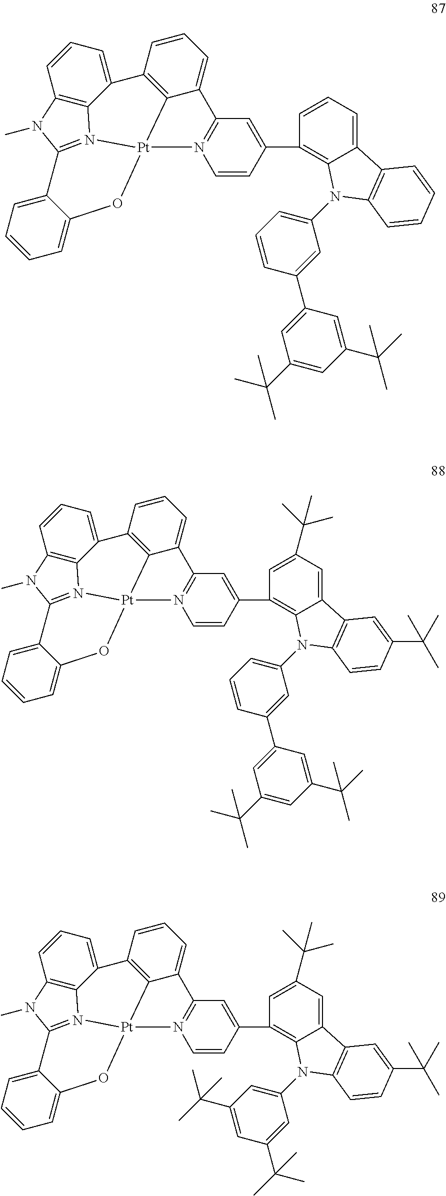

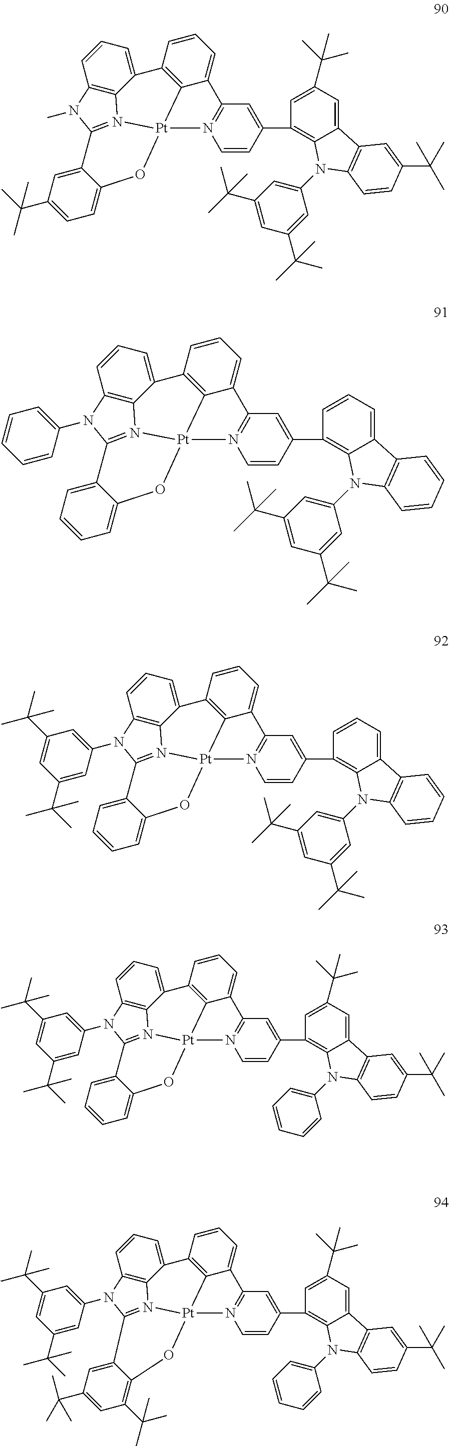

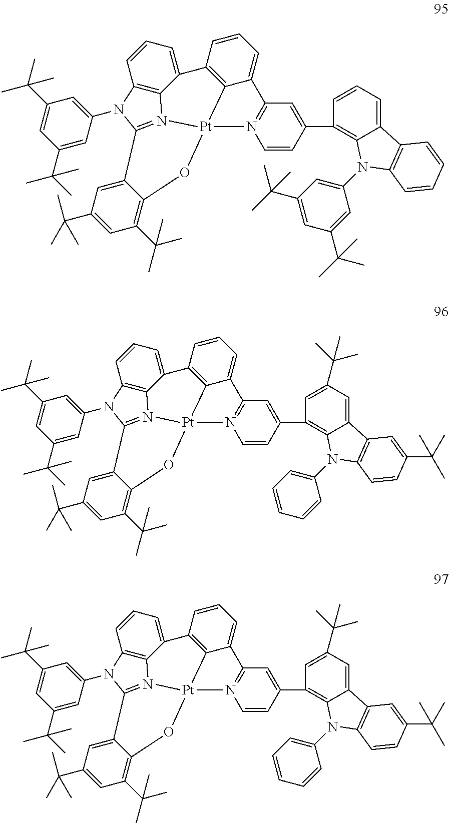

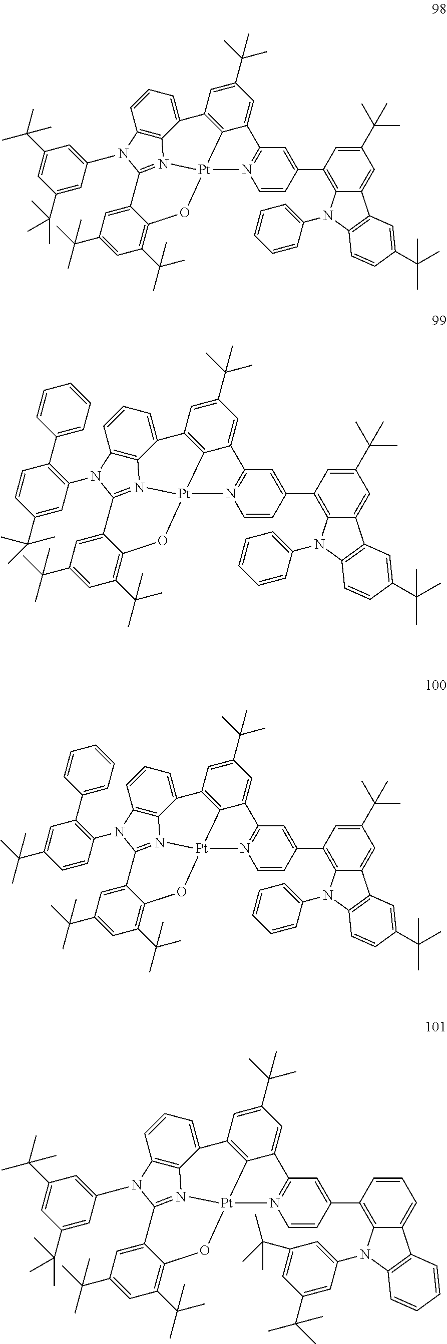

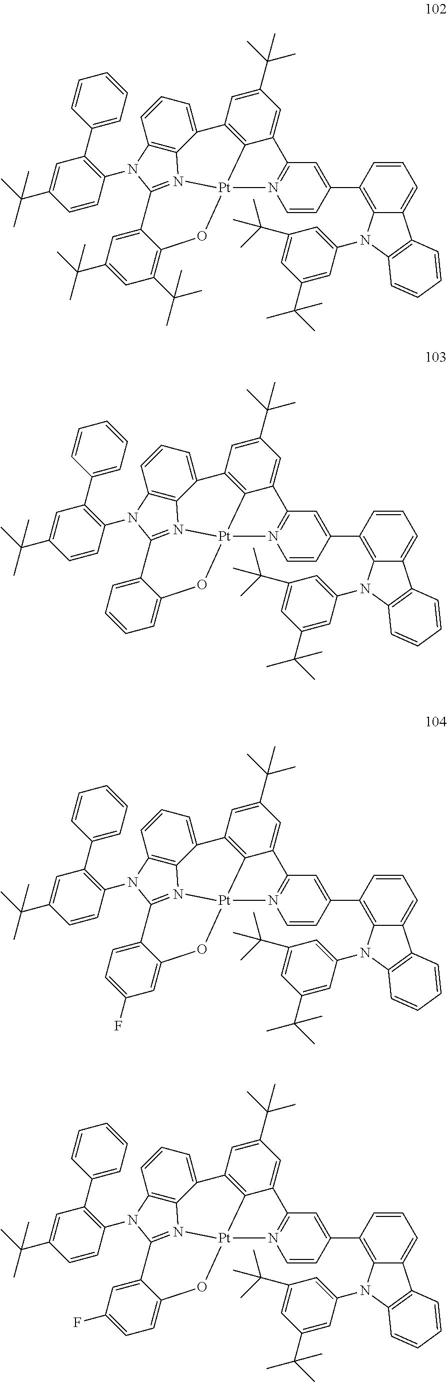

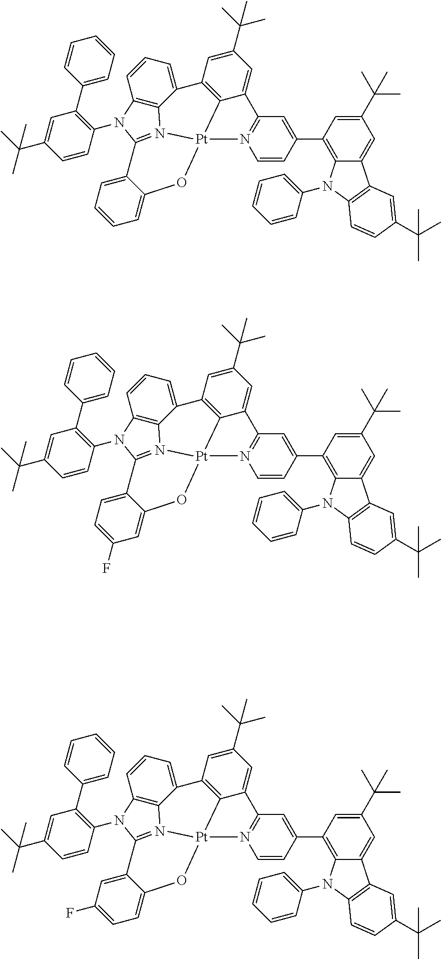

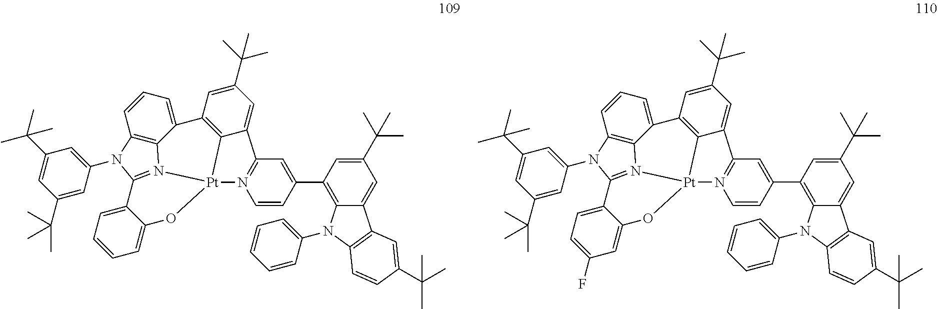

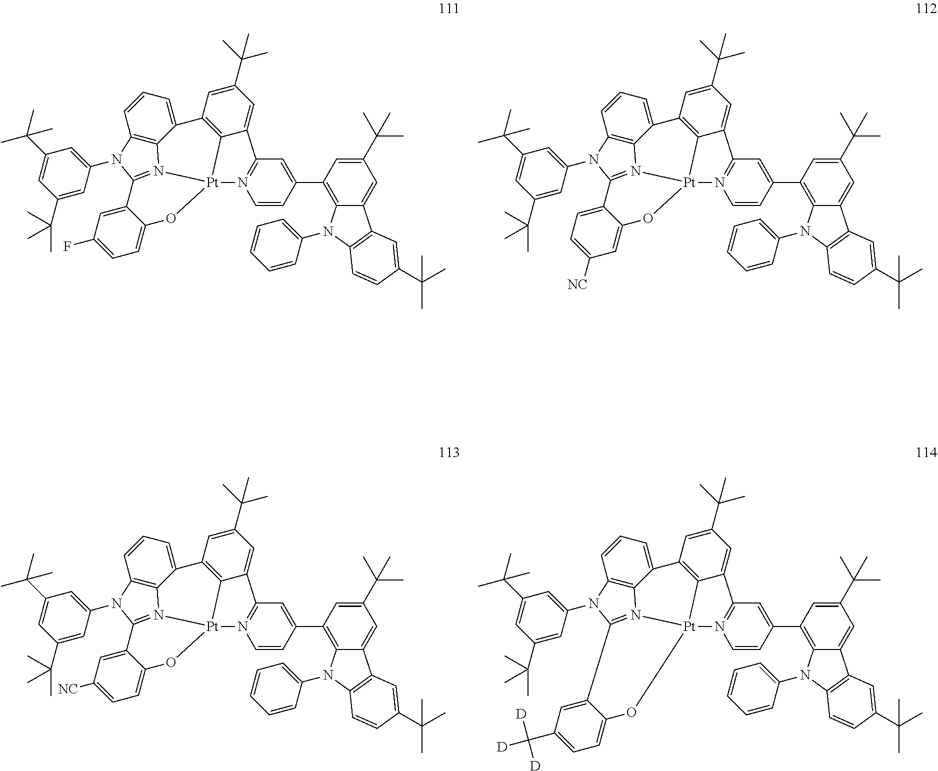

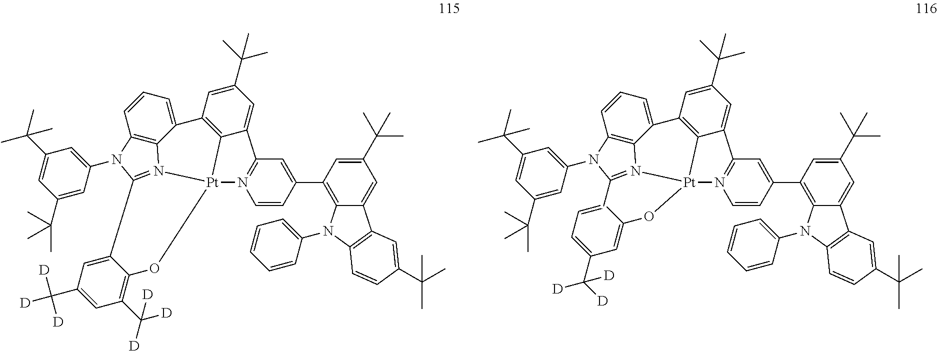

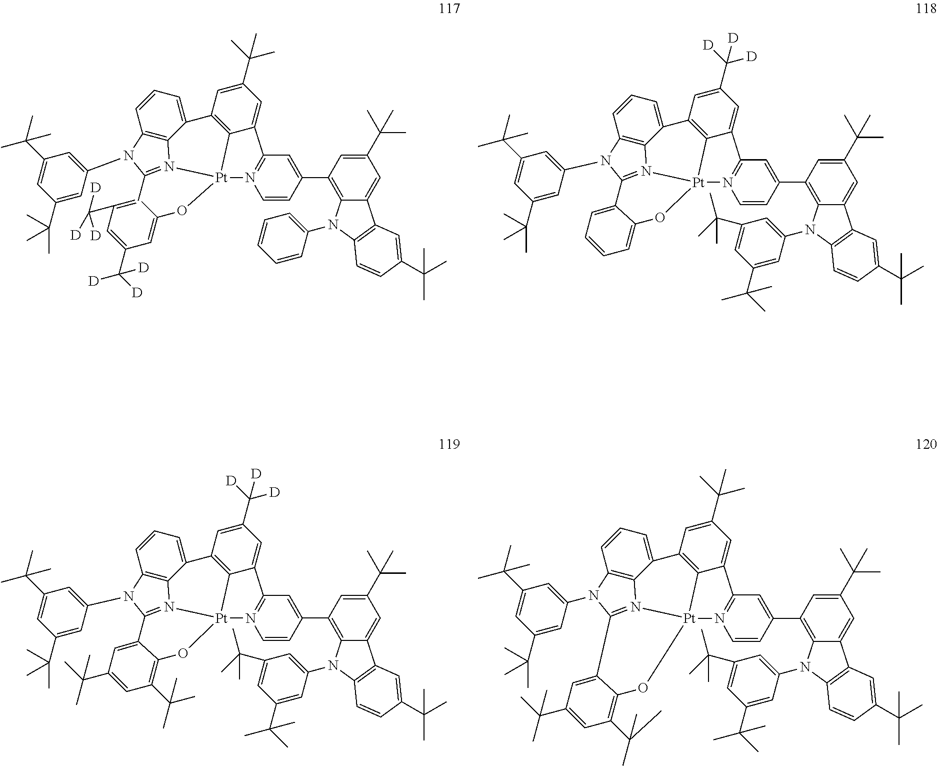

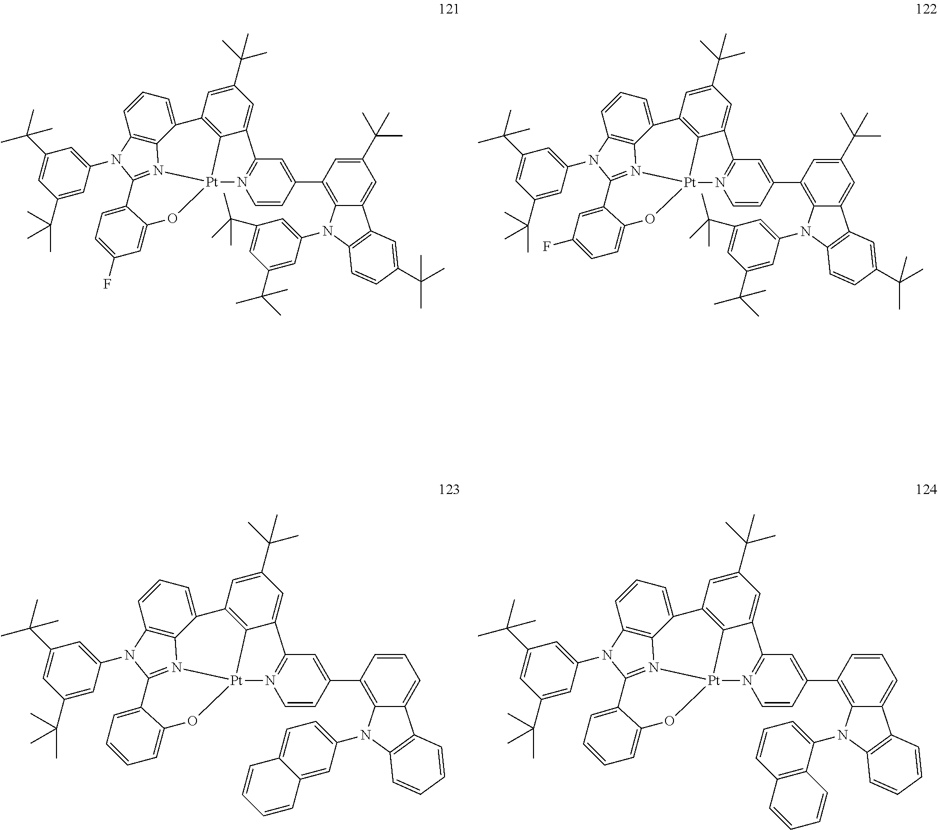

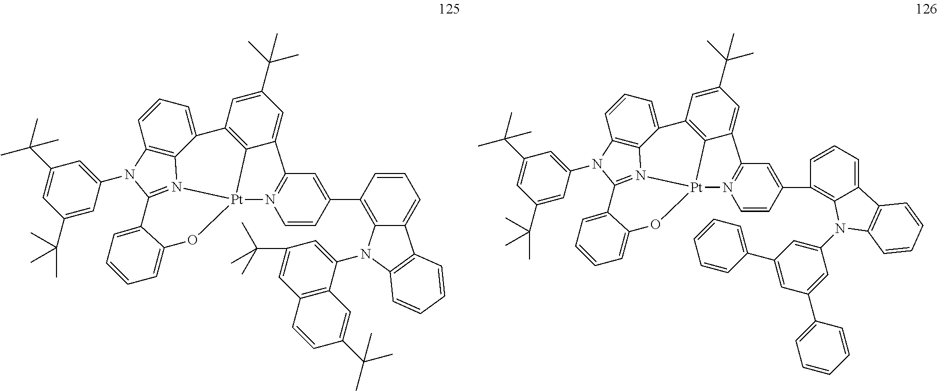

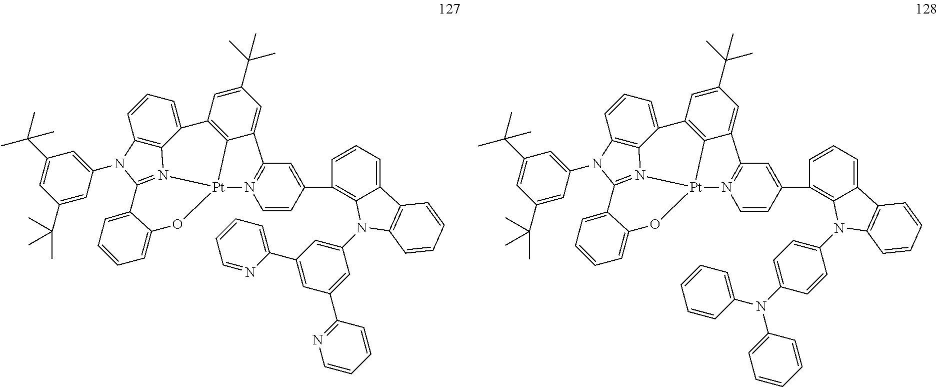

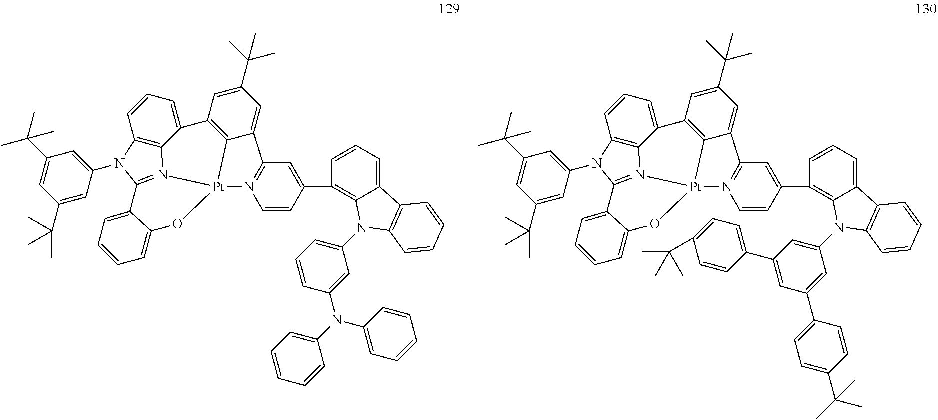

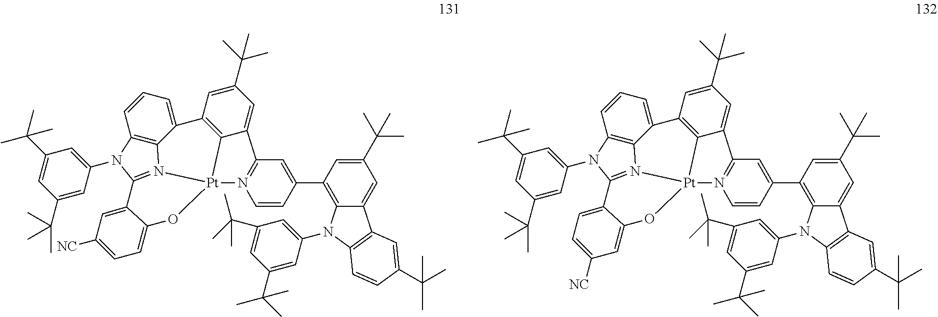

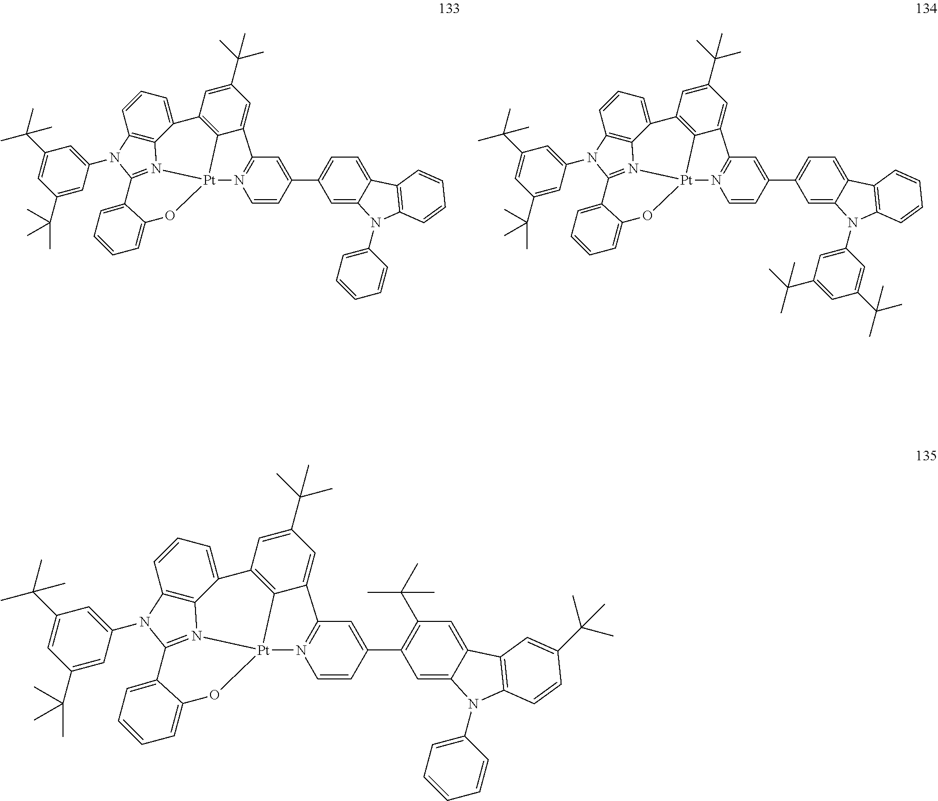

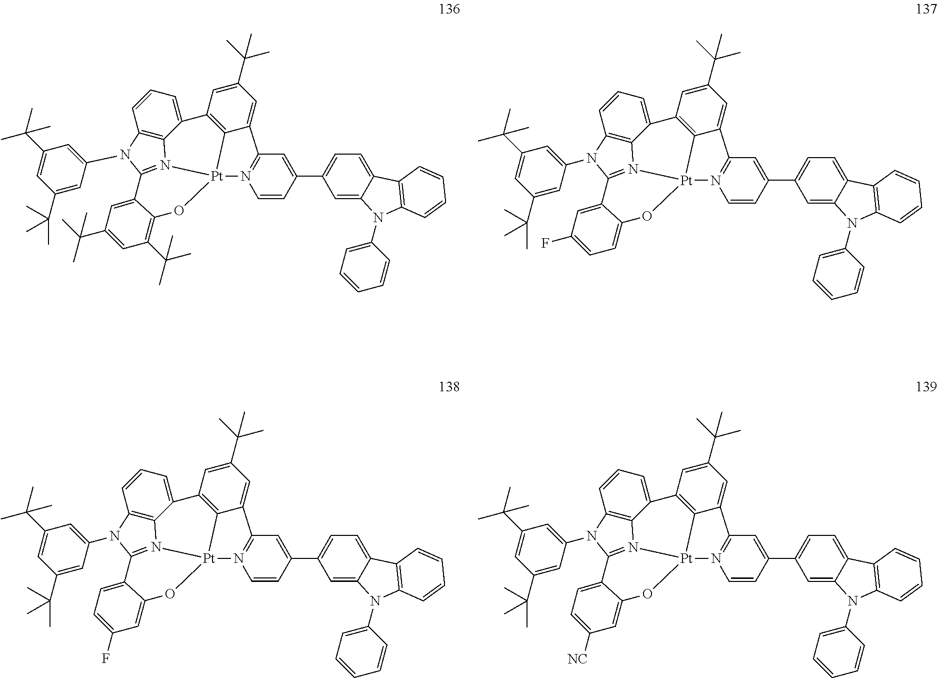

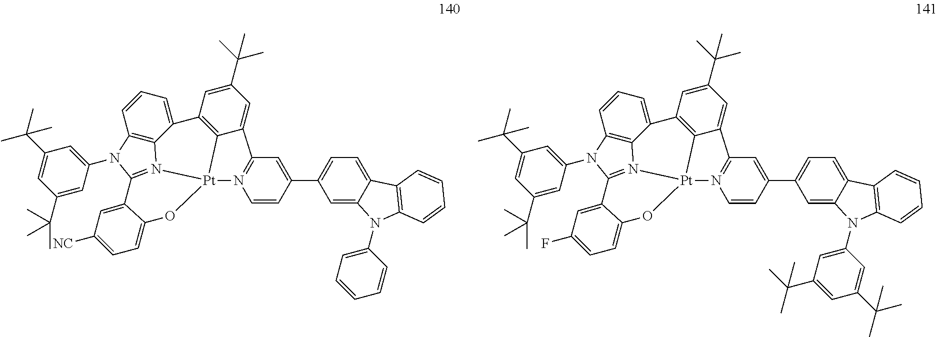

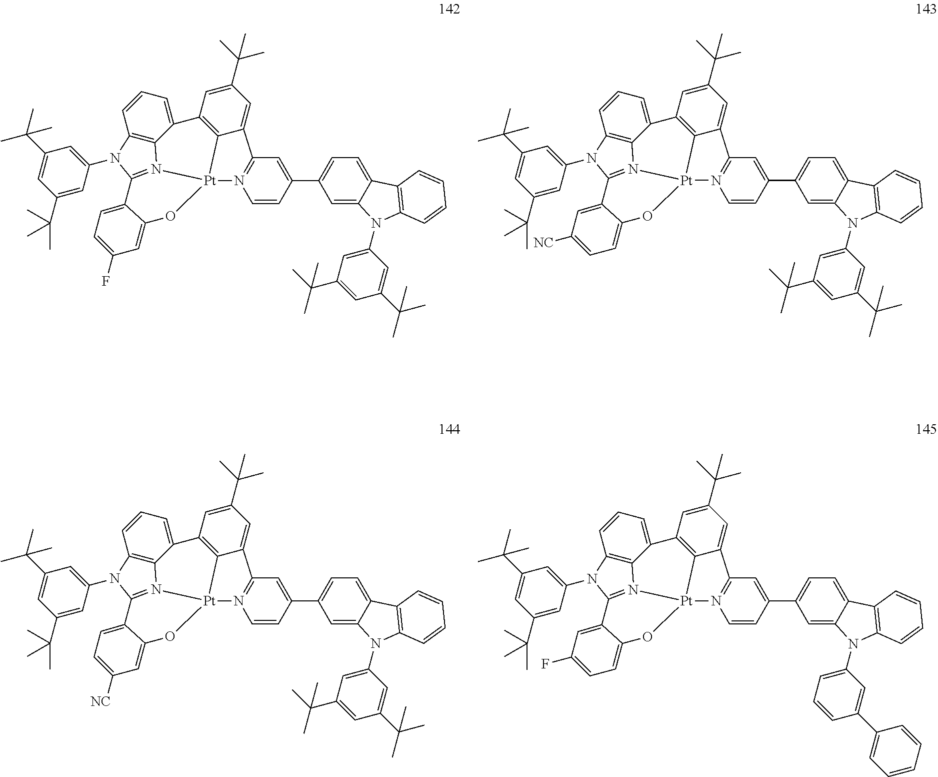

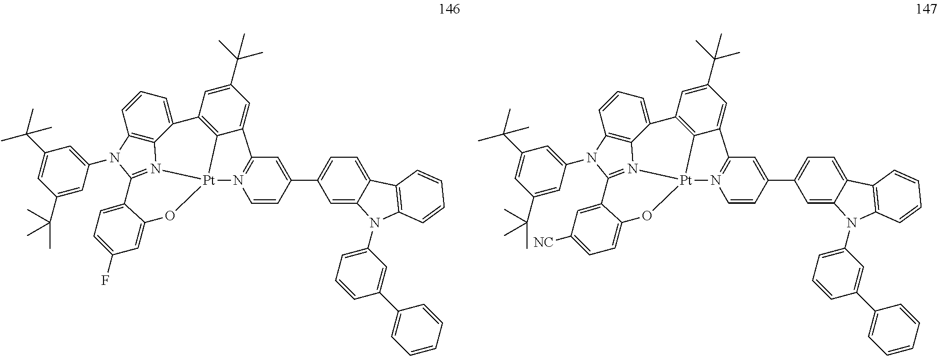

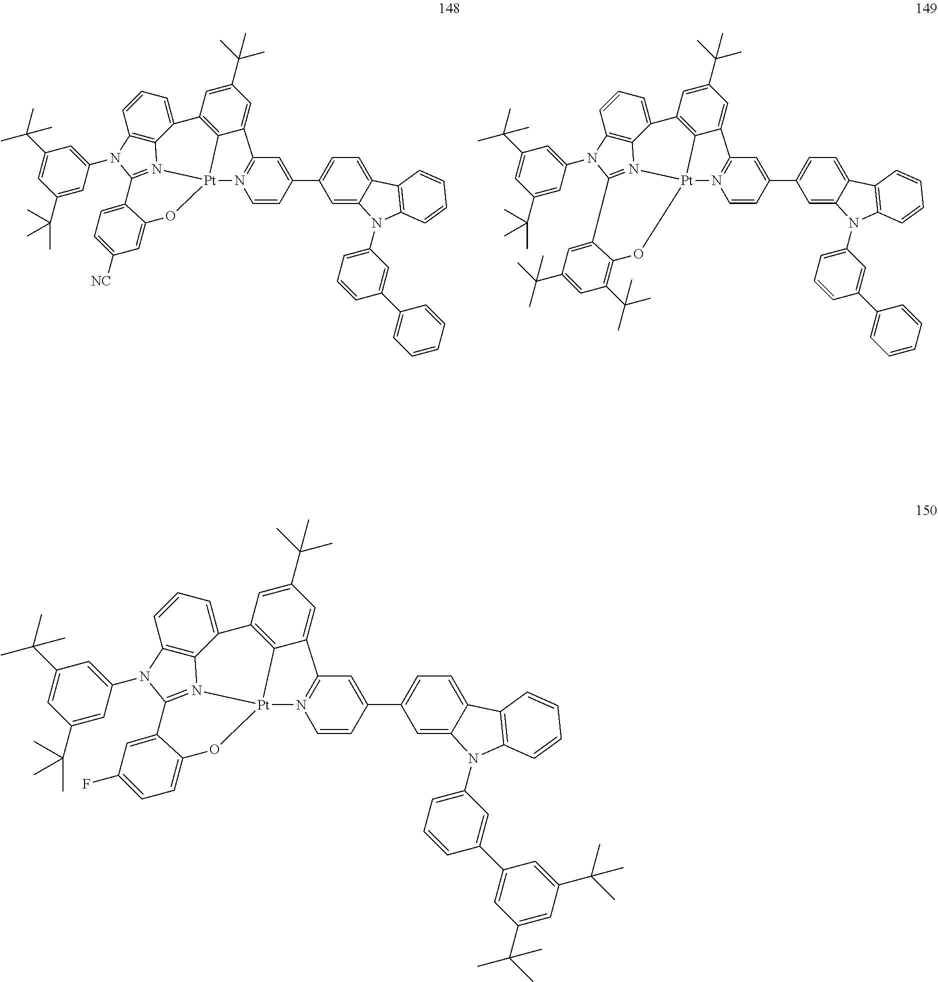

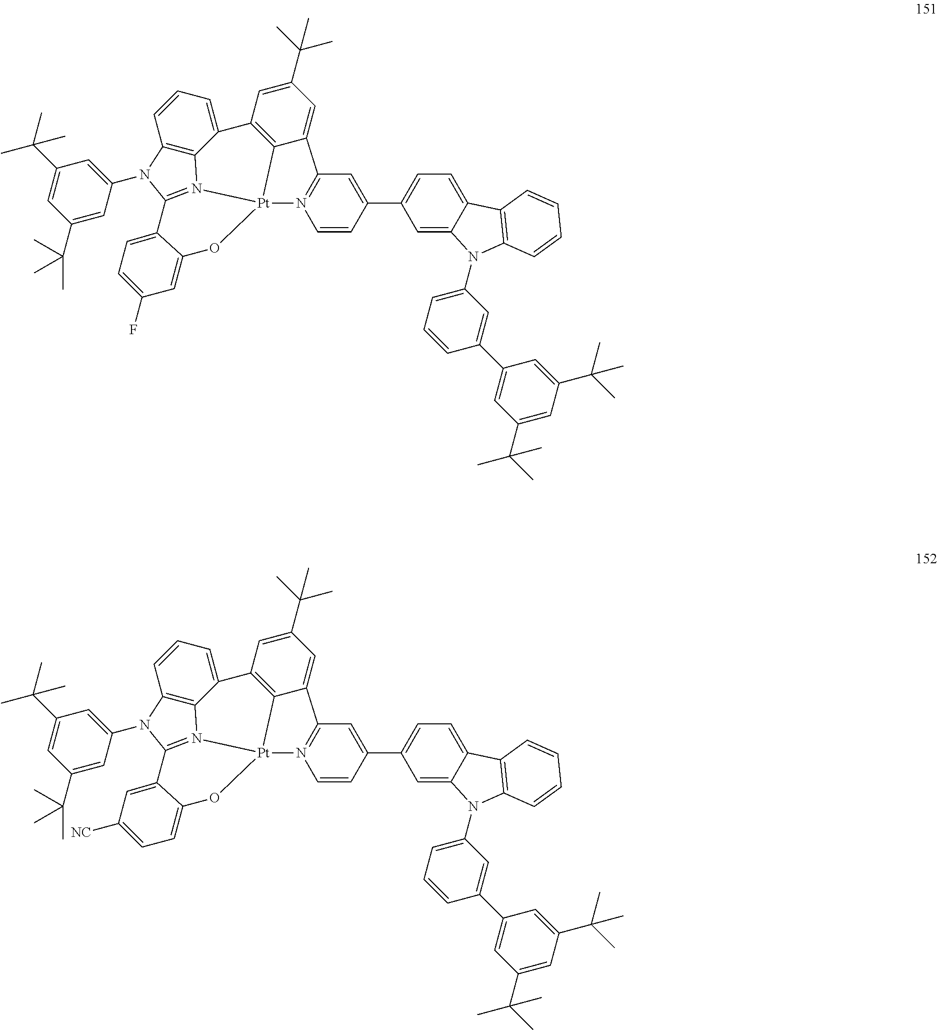

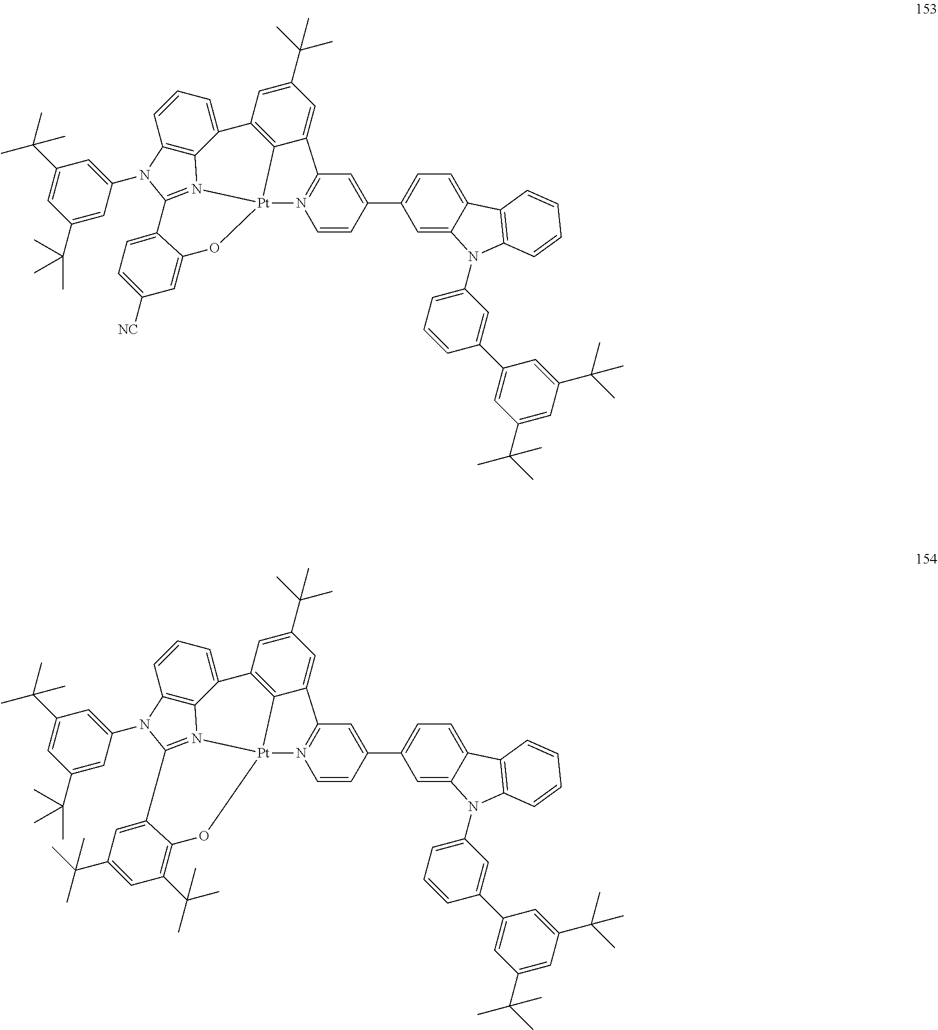

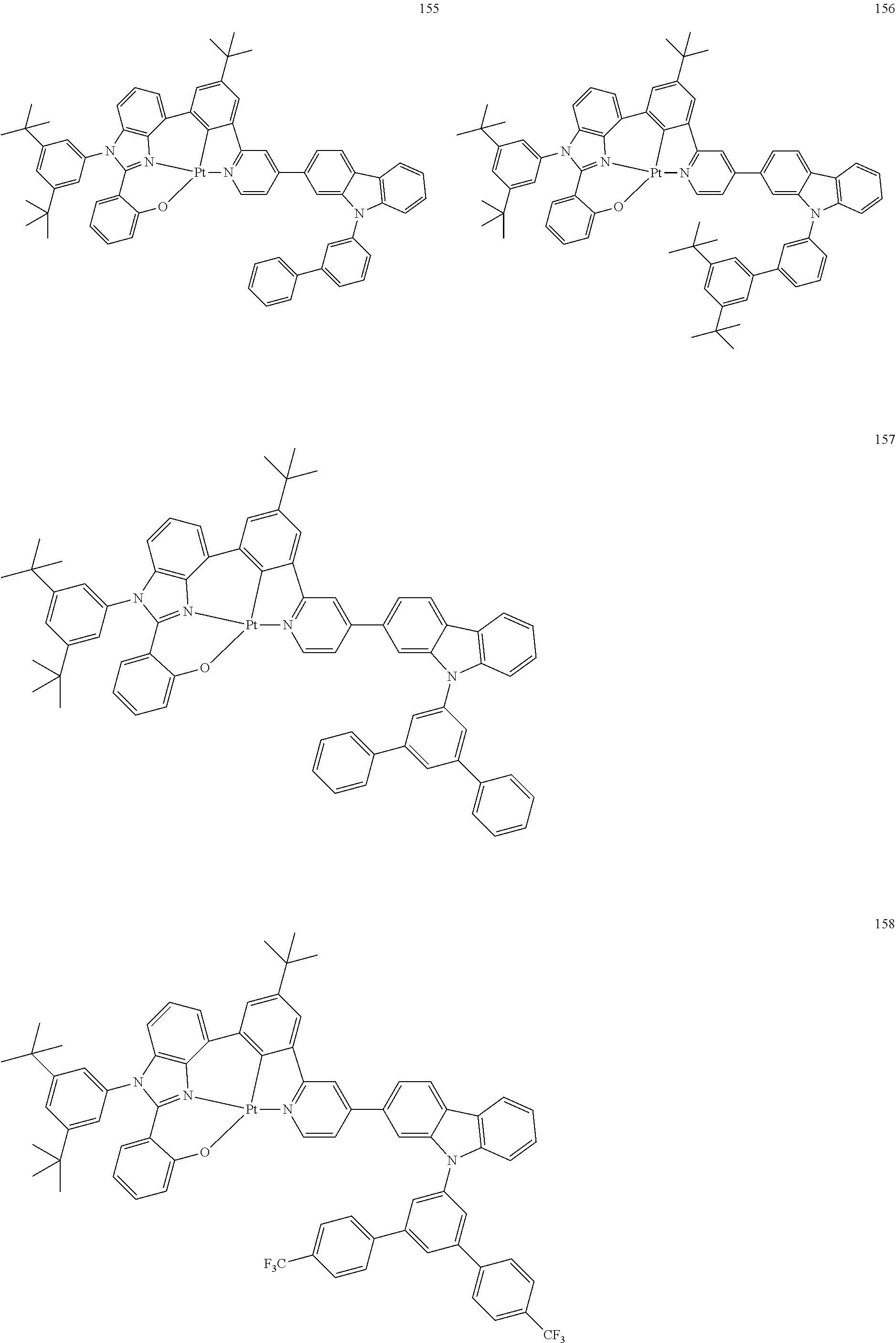

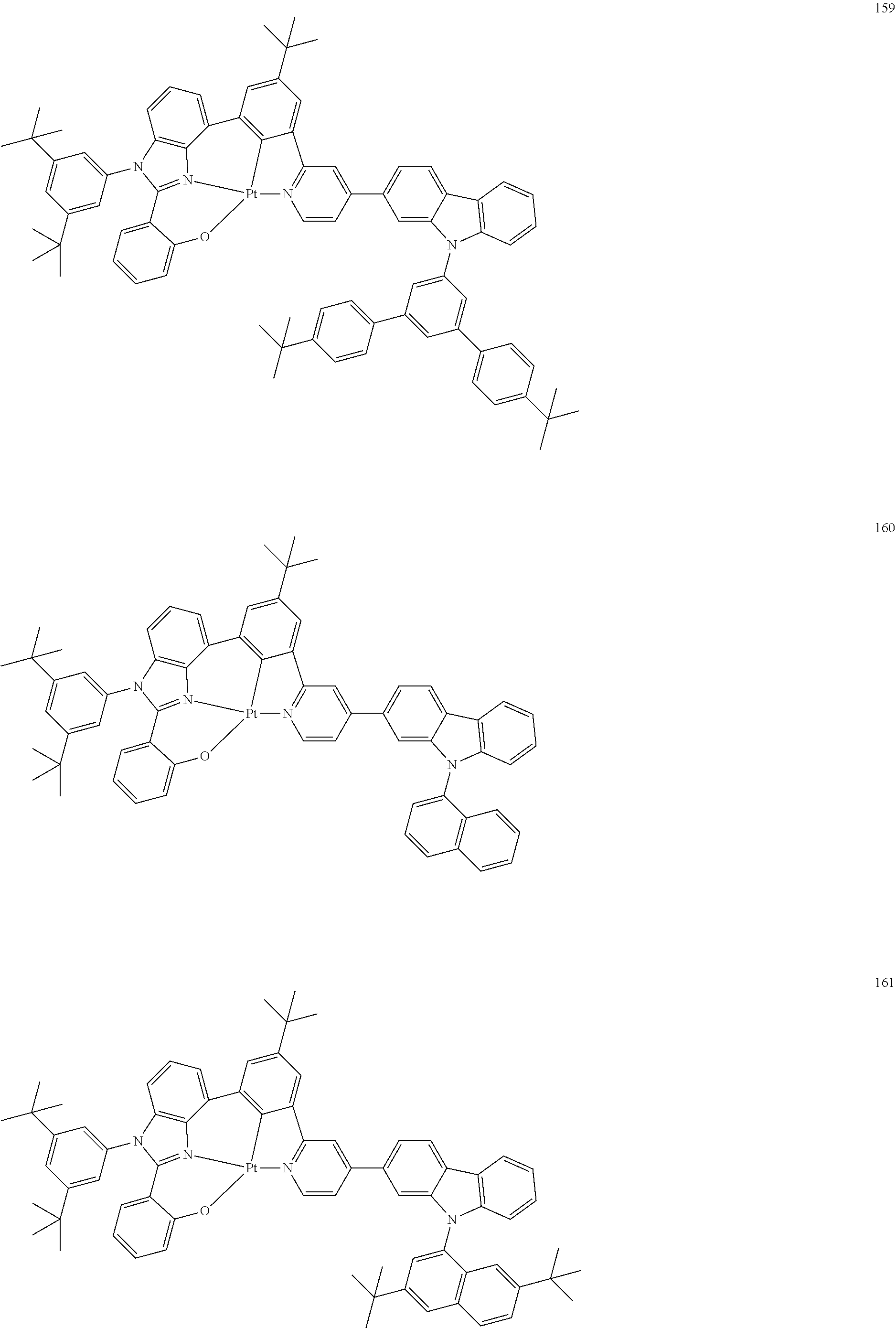

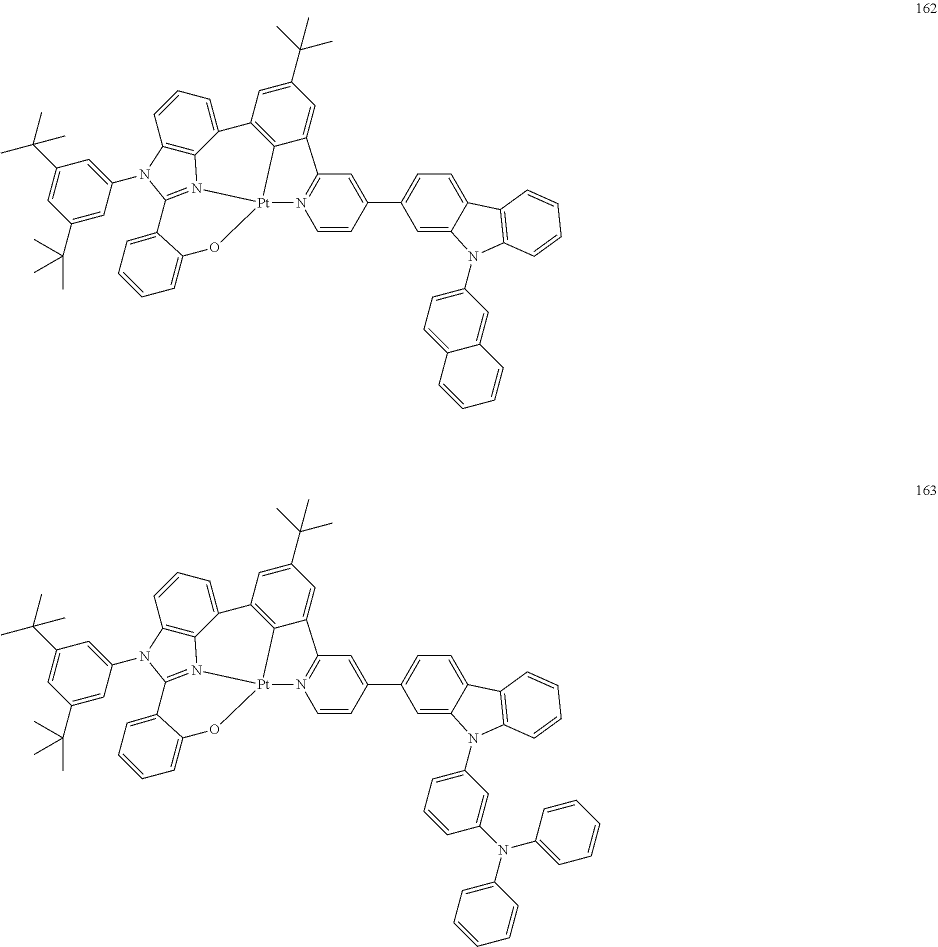

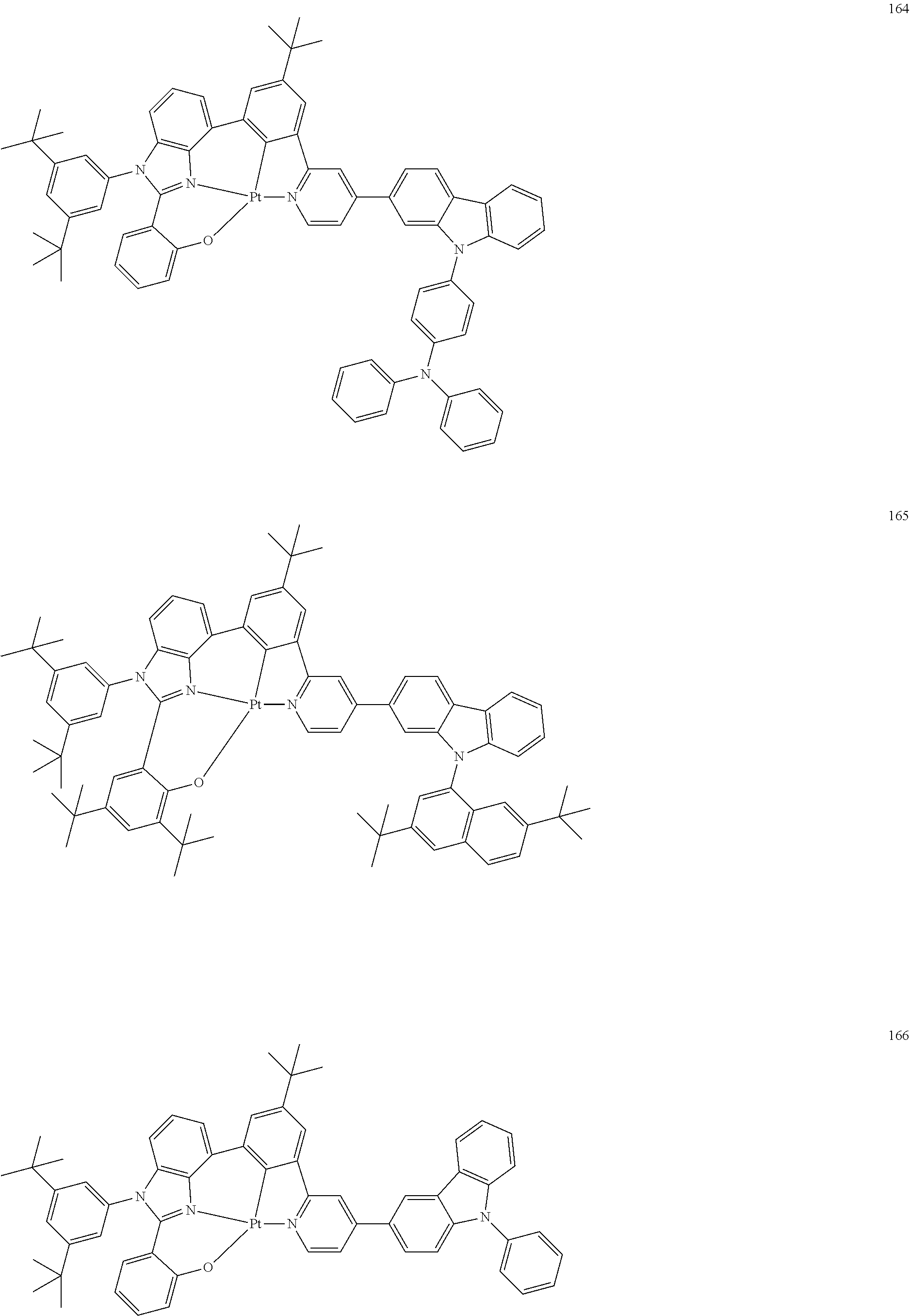

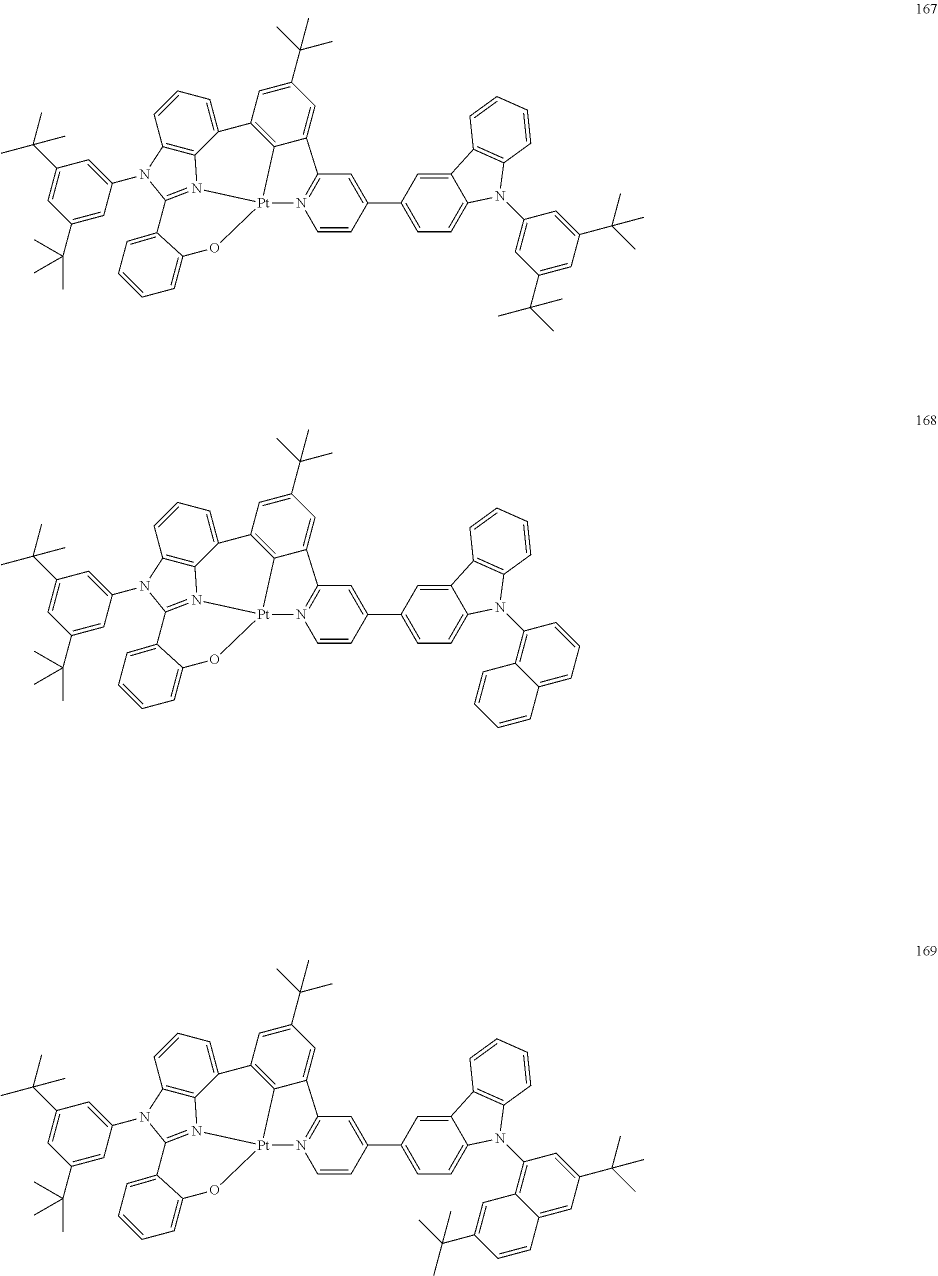

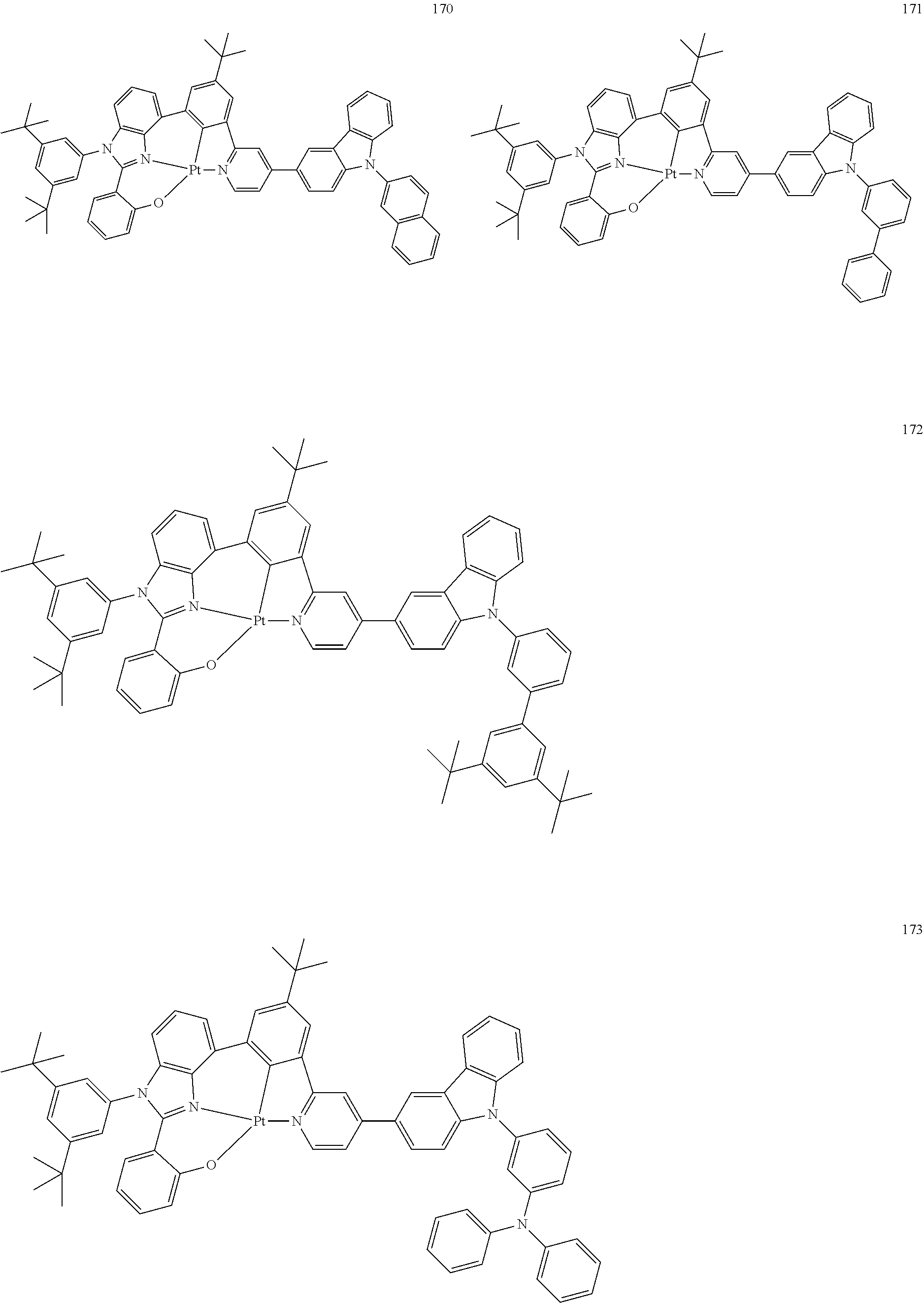

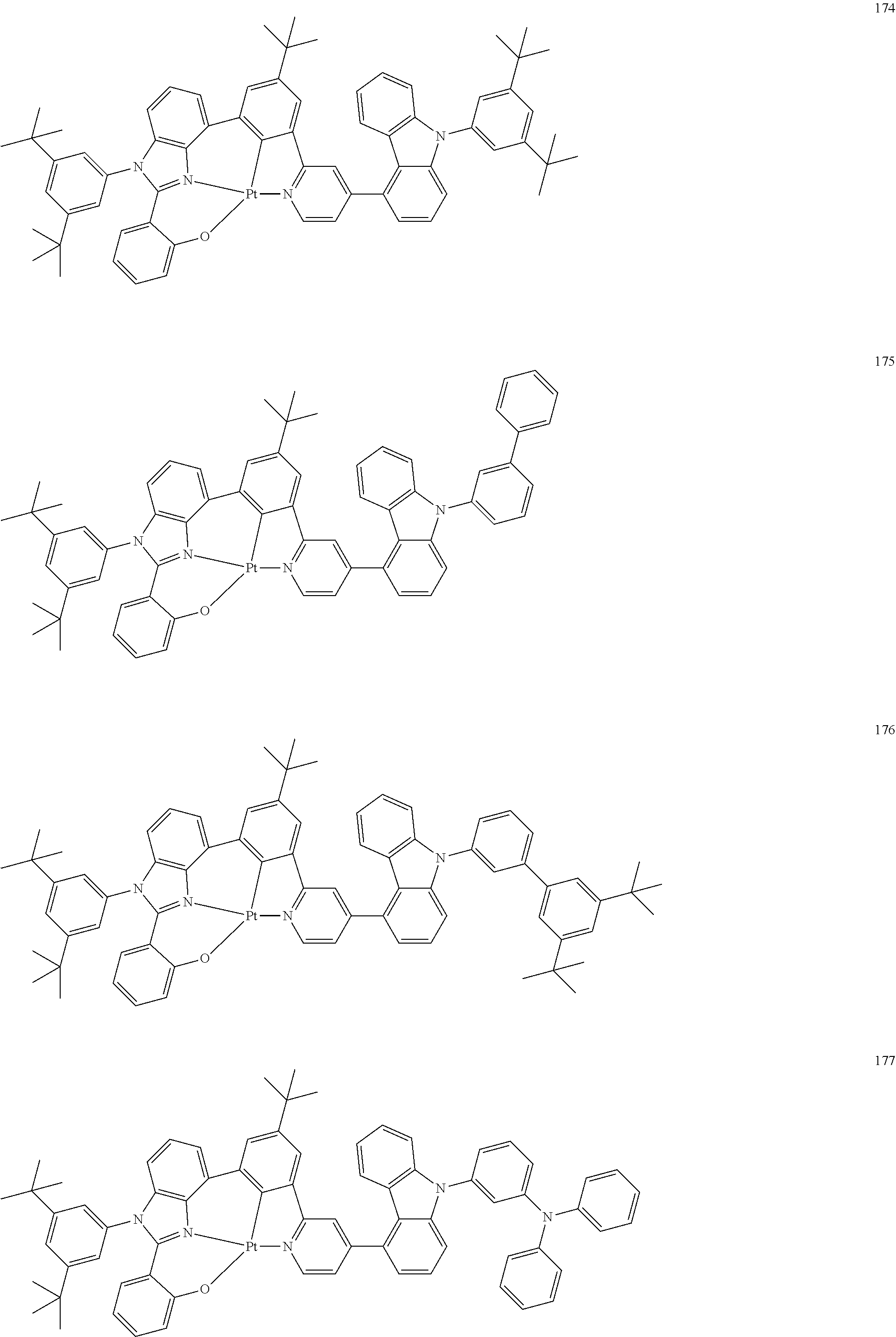

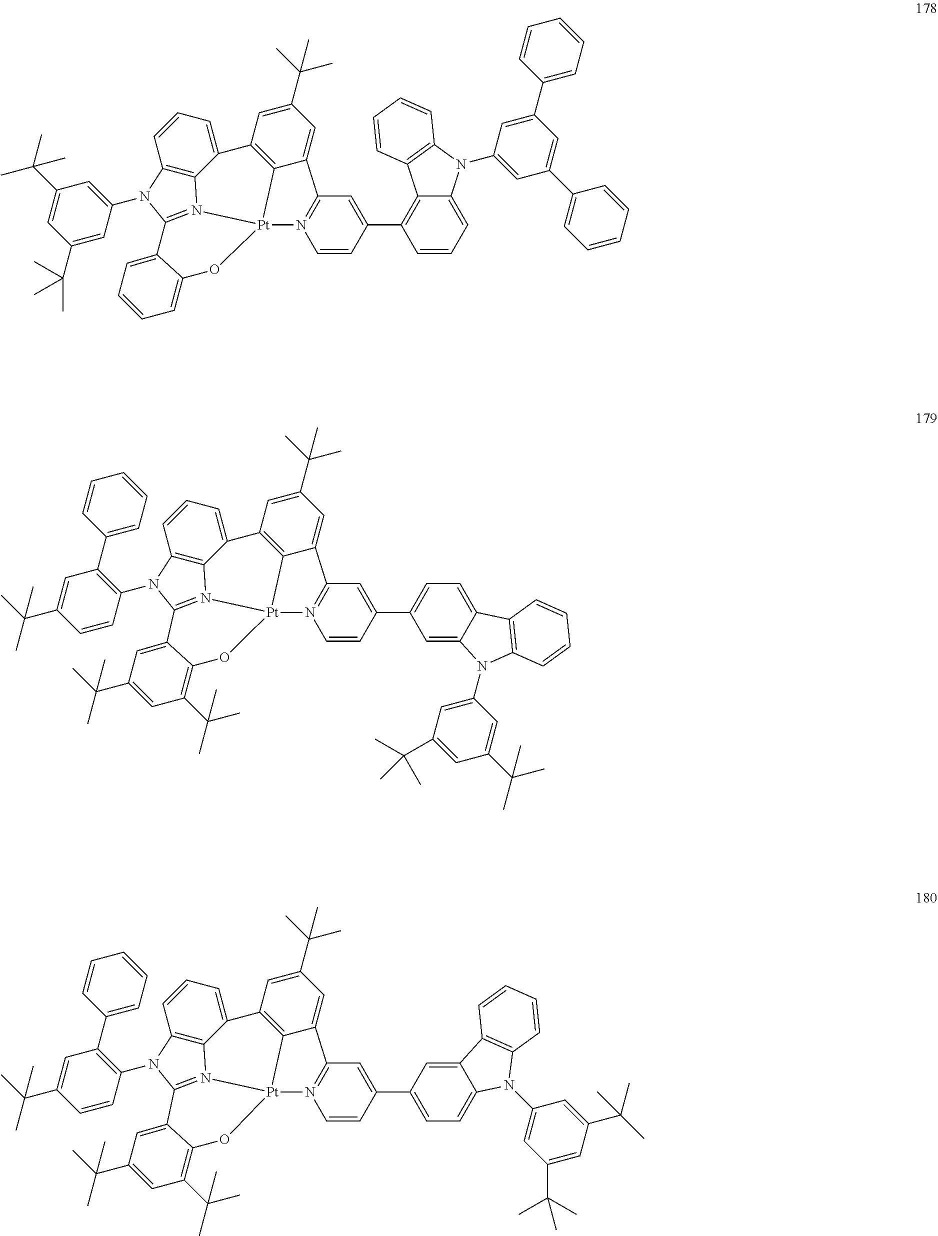

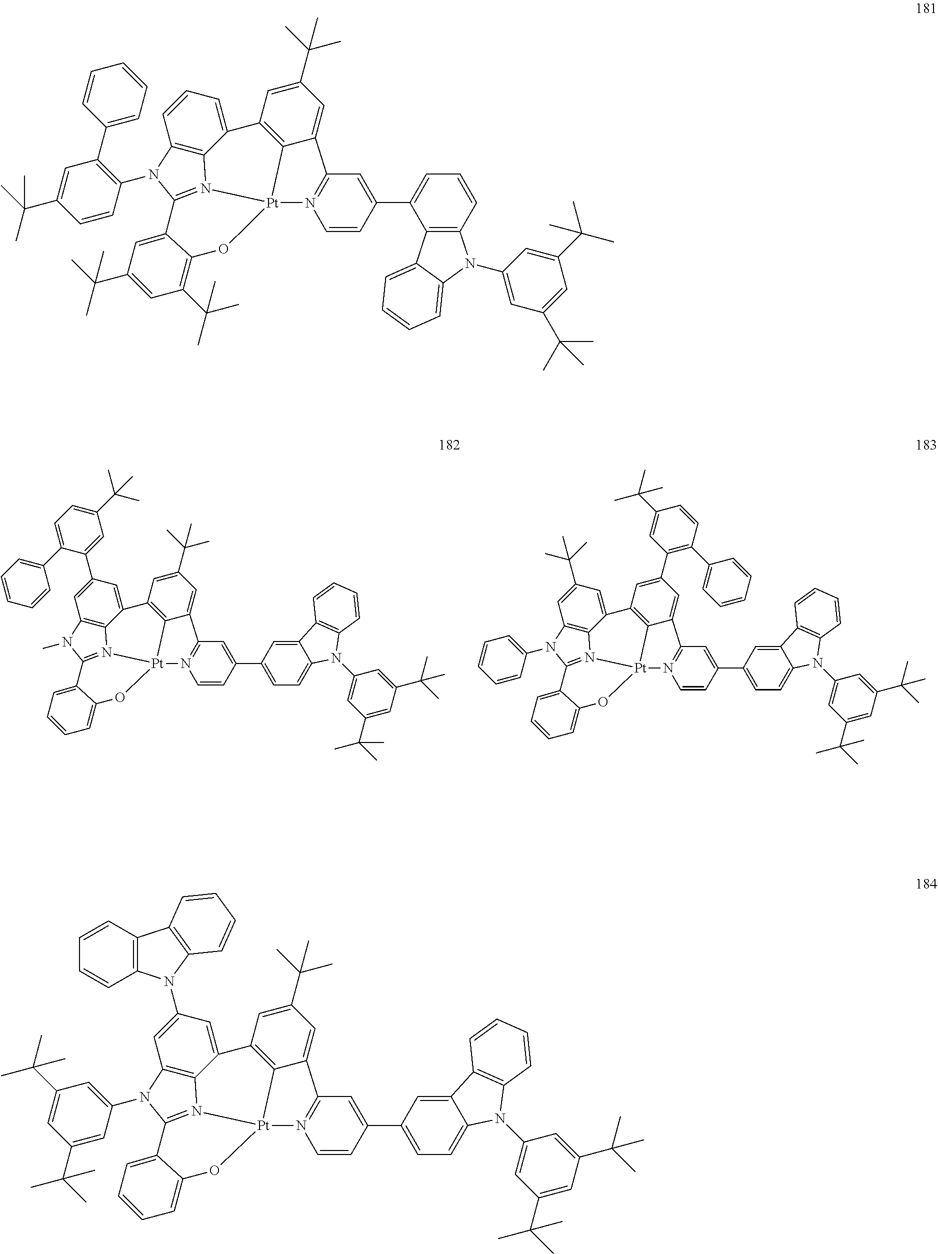

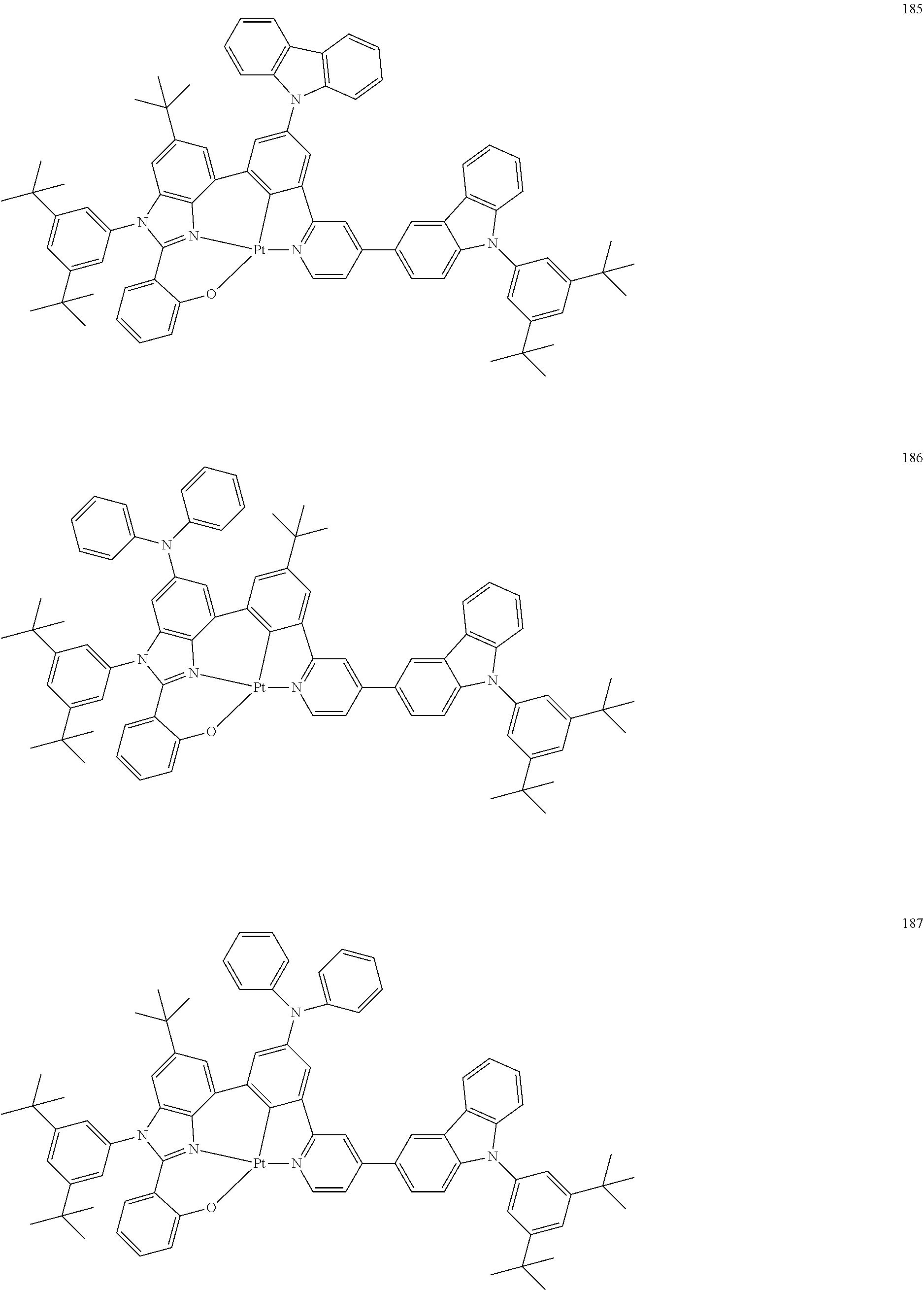

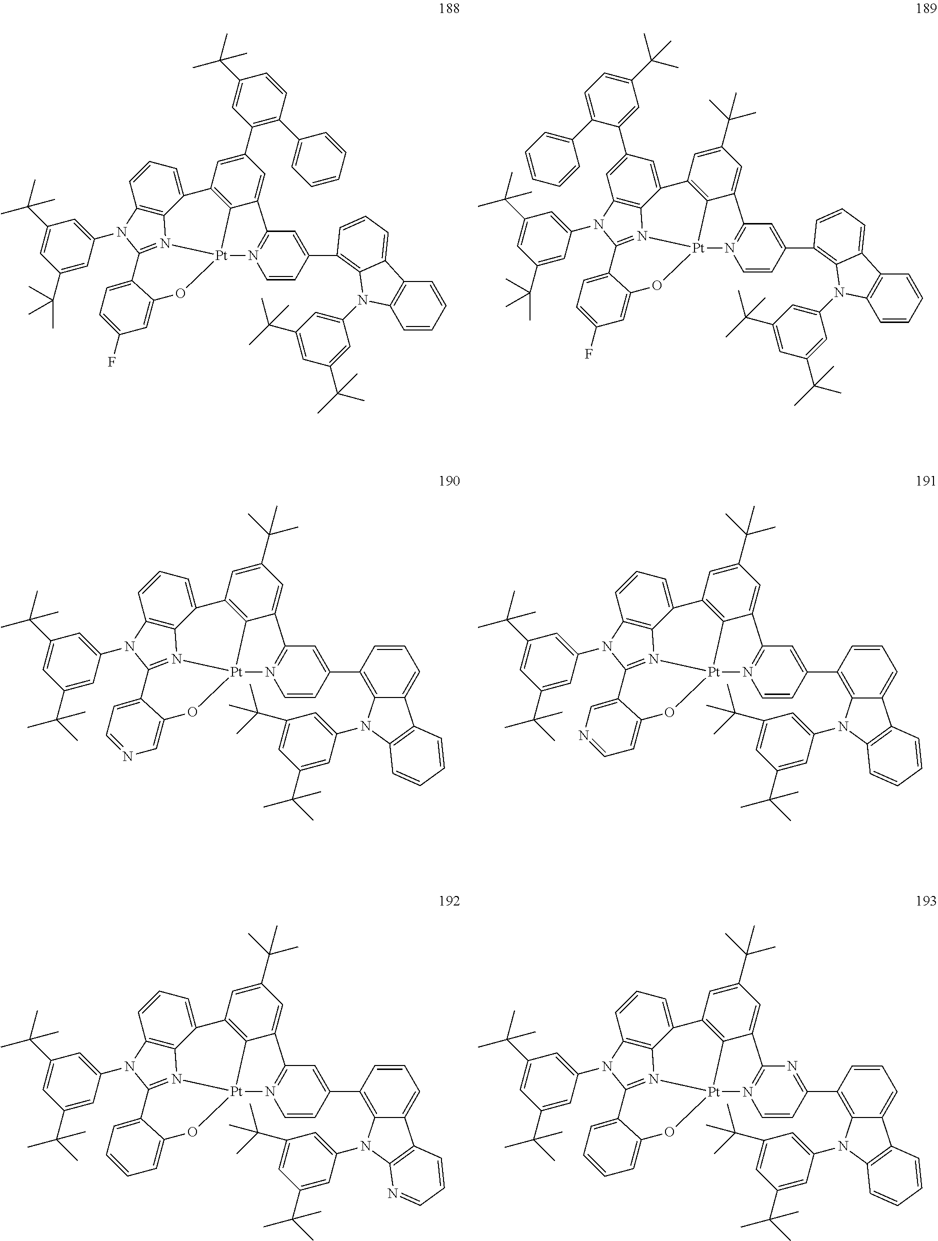

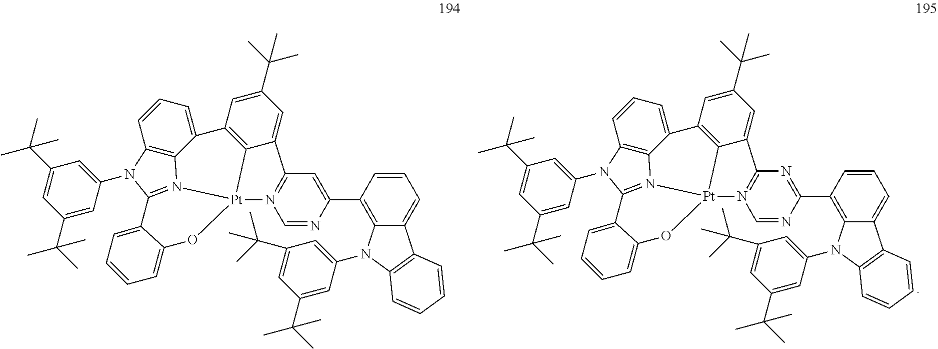

A precursor, i.e., a ligand, of the above-mentioned metal complex has the following structural formula:

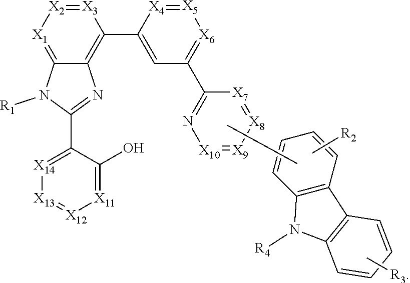

The present application further provides an application of the above-mentioned platinum complex in an organic photoelectronic device. The photoelectronic device includes, but is not limited to, an organic light-emitting diode, an organic thin film transistor, an organic photovoltaic device, a luminescent electrochemical cell, and a chemical sensor, preferably, an organic light-emitting diode.

The organic light-emitting diode in the present application includes a cathode, an anode, and an organic layer. The organic layer includes one or more of a hole injection layer, a hole transport layer, a light-emitting layer, a hole blocking layer, an electron injection layer, or an electron transport layer, and it is not necessary for each of these organic layers to be present. At least one of the hole injection layer, the hole transport layer, the hole blocking layer, the electron injection layer, the light-emitting layer, or the electron transport layer includes the platinum complex of formula (I).

In an embodiment, the layer including the platinum complex of formula (I) is the light-emitting layer or the electron transport layer.

The organic layer in the device of the present application has a total thickness of 1 nm to 1000 nm, preferably 1 nm to 500 nm, and more preferably 5 nm to 300 nm.

The organic layer can be a thin film formed by an evaporation method or a solution method.

The present application discloses a series of platinum complex luminescent materials which has good luminescent property and can be applied in an organic light-emitting diode as a luminescent material.

The platinum complex, when used in the organic light-emitting diode, exhibits a low driving voltage and a high luminous efficiency with a significantly expanded service life of the device, having potential to be applied in the field of organic electroluminescent devices.

### BRIEF DESCRIPTION OF THE DRAWING

The FIGURE shows a structural diagram of an organic light-emitting diode device of the present application.

In the FIGURE, 10 denotes a glass substrate, 20 denotes an anode, 30 denotes a hole injection layer, 40 denotes a hole transport layer, 50 denotes a light-emitting layer, 60 denotes an electron transport layer, 70 denotes an electron injection layer, and 80 denotes a cathode.

### DETAILED DESCRIPTION

### Example 1: Synthesis of Compound C

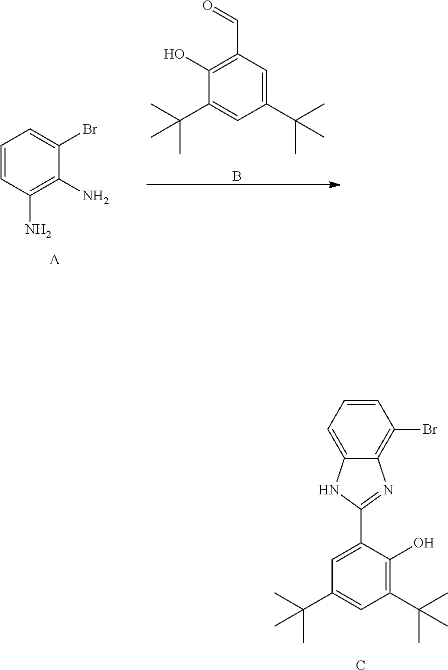

The compound A (10.0 g, 53 mmol, 1.0 eq), the compound B (12.5 g, 53 mmol, 1.0 eq), Na₂S₂O₅ (508 mg, 2.6 mmol, 0.05 eq), and DMF (200 mL) were added into a 250 mL single-necked flask with N₂ introduced to carry out a reaction at 80° C. for 4 h. After the reaction was finished, the reaction liquid was spotted on a chromatoplate with Hex/EA=10/1 (V/V) as developing solvent, and the target compound was found, suggesting that the reaction was complete and the reaction liquid was ready for post-processing. The reaction liquid was extracted with water (100 mL)+EA (30 mL) to achieve a phase separation to obtain an EA layer. The EA layer was concentrated and separated by silica gel column (Hex/EA=10/1 (V/V)) to obtain 2.9 g of yellow solid with a yield of 13.51% and a HPLC purity of 99.64%. The H-NMR data is as follows: ¹H NMR (400 MHz, CDCl₃) δ 7.75 (d, J=2.0 Hz, 1H), 7.64 (s, 1H), 7.55 (s, 1H), 7.47 (s, 1H), 7.04 (d, J=2.0 Hz, 1H), 1.42 (s, 9H), 1.36 (s, 9H).

### Example 2: Synthesis of Compound K

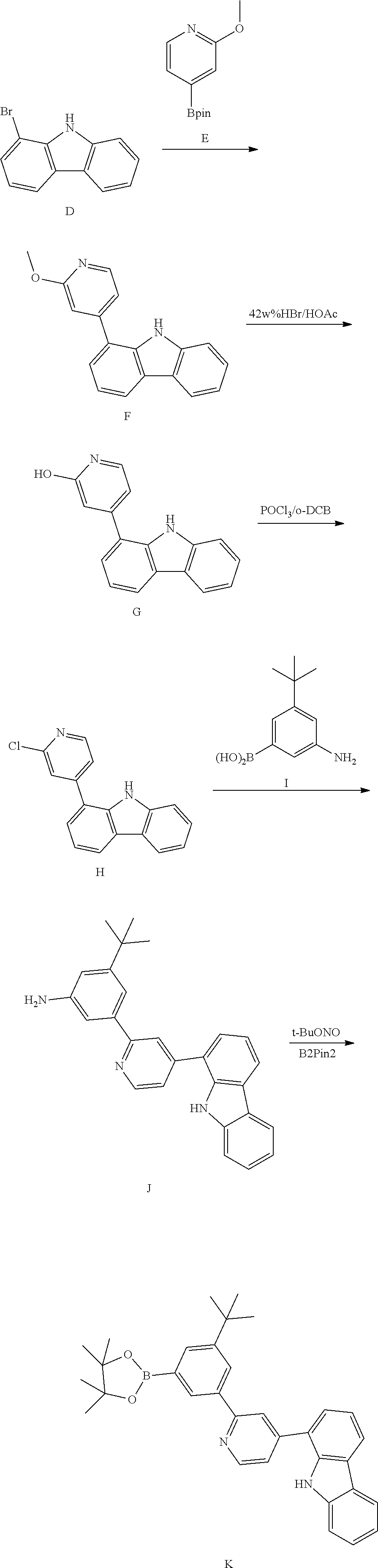

### Synthesis of Compound F

The compound D (10.0 g, 40 mmol, 1.0 eq.), the compound E (11.7 g, 50 mmol, 1.25 eq.), Pd₁₃₂ (280 mg, 0.4 mmol, 1% eq.), K₂CO₃ (13.8 g, 100 mmol, 2.5 eq.), and toluene/ethanol/H₂O (200/200/50 ml) were added into a 1000 ml three-necked flask to carry out a reaction at 90° C. for 12 h under stirring and protection of nitrogen gas. After the reaction was finished, the reaction liquid was rotary-evaporated to remove most of the solvent, added with deionized water, extracted with dichloromethane for three times, rotary-evaporated, and separated by silica gel column (Hex:EA=10:1) to obtain 21.0 g of brown solid with a yield of 94.2%. The H-NMR data is as follows: ¹H NMR (400 MHz, CDCl₃) δ 8.52 (s, 1H), 8.31 (d, J=5.2 Hz, 1H), 8.16-8.07 (m, 2H), 7.46 (dd, J=9.1, 2.6 Hz, 3H), 7.31 (s, 1H), 7.28 (s, 1H), 7.20 (dd, J=5.3, 1.4 Hz, 1H), 7.08 (s, 1H), 4.03 (s, 3H).

### Synthesis of Compound G

The compound F (2.0 g, 7.2 mmol, 1.0 eq.), pyridine hydrochloride (10 g, with a mass ratio of 5:1 to the compound F), and o-dichlorobenzene (o-DCB, 2 ml) were added into a 250 mL single-necked flask to carry out a reaction at 180° C. for 3.5 h under protection of nitrogen gas. After the reaction was finished, the reaction liquid was cooled to room temperature, added with water and dichloromethane, stirred for 30 min, and subjected to phase separation to collect an organic layer to obtain a crude product. The crude product was triturated with hexane (Hex) to obtain 2.0 g of yellow solid with a yield of about 100%. ¹H NMR (400 MHz, DMSO) δ 11.22 (s, 1H), 8.15 (dd, J=18.5, 7.7 Hz, 2H), 7.59-7.52 (m, 2H), 7.46-7.36 (m, 2H), 7.24 (t, J=7.6 Hz, 1H), 7.20-7.13 (m, 1H), 6.67 (d, J=1.2 Hz, 1H), 6.52 (dd, J=6.7, 1.7 Hz, 1H).

### Synthesis of Compound H

The compound G was added with POCl₃ (10 mL) and O-DCB (1 ml) to carry out a reaction at 100° C. for 18 h under protection of nitrogen gas. After the reaction was finished, the reaction liquid was cooled to room temperature, rotary-evaporated to remove a part of POCl₃ until viscous, added with ice water, stirred to completely quench POCl₃, and extracted with dichloromethane to obtain a crude product. The crude product was triturated with Hex to obtain 2.0 g of yellow solid with a yield of about 100%. The H-NMR data is as follows: ¹H NMR (400 MHz, CDCl₃) δ 8.59 (s, 1H), 8.49 (dd, J=5.1, 0.6 Hz, 1H), 8.14 (dd, J=17.2, 7.5 Hz, 2H), 7.70-7.65 (m, 1H), 7.55 (dd, J=5.1, 1.5 Hz, 1H), 7.53-7.42 (m, 3H), 7.35 (t, J=7.6 Hz, 1H), 7.32-7.27 (m, 1H).

### Synthesis of Compound J

The compound H (2.0 g, 7.17 mmol, 1.0 eq.), the compound I, (11.7 g, 8.97 mmol, 1.25 eq.), Pd₁₃₂ (51 mg, 0.072 mmol, 1% eq.), K₂CO₃ (2.48 g, 100 mmol, 2.5 eq.), and toluene/ethanol/H₂O (50/50/10 ml) were added into a 250 mL single-necked flask to carry out a reaction at 90° C. for 12 h under stirring and protection of nitrogen gas. After the reaction was finished, the reaction liquid was rotary-evaporated to remove most of the solvent, added with deionized water, extracted with dichloromethane for three times, rotary-evaporated, and separated by silica gel column (Hex:EA=10:1) to obtain 2.5 g of brown solid with a yield of 89.3%. The H-NMR data is as follows: ¹H NMR (400 MHz, CDCl₃) δ 8.58 (d, J=4.9 Hz, 1H), 8.15 (d, J=12.7 Hz, 2H), 7.89-7.82 (m, 2H), 7.66 (t, J=2.0 Hz, 1H), 7.54 (s, 1H), 7.50 (s, 1H), 7.38 (dd, J=5.0, 1.0 Hz, 1H), 7.25 (d, J=13.0 Hz, 2H), 7.20 (t, J=2.0 Hz, 1H), 6.61 (t, J=2.0 Hz, 1H), 4.29 (s, 2H), 1.36 (s, 9H).

### Synthesis of Compound K

Tert-butyl nitrite (155 mg, 1 mmol) was added dropwise to a mixture of bis(pinacolato)diboron (127 mg, 0.5 mmol), the compound J (195 mg, 0.5 mmol), and eosin Y (0.01 mmol) in acetonitrile (5 mL). The resulting mixture was stirred at room temperature under blue LED irradiation for 2 hours (TLC). The mixture diluted with ethyl acetate (EA, 5 mL) was filtered through diatomaceous earth and the filtrate was extracted with ethyl acetate (3×10 mL). The extract was washed with brine, dried over anhydrous Na₂SO₄, and evaporated to obtain a crude product. The crude product was purified by silica gel column chromatography (Hex:EA=10:1) to obtain 208 mg of brown solid with a yield of 88%. The H-NMR data is as follows: ¹H NMR (400 MHz, CDCl₃) δ 8.57 (d, J=5.0 Hz, 1H), 8.15 (d, J=12.7 Hz, 2H), 7.84 (d, J=1.4 Hz, 2H), 7.67 (t, J=1.9 Hz, 1H), 7.54 (s, 1H), 7.50 (s, 1H), 7.45 (dt, J=8.1, 2.0 Hz, 2H), 7.38 (dd, J=5.0, 1.0 Hz, 1H), 7.25 (d, J=13.0 Hz, 2H), 1.38 (s, 12H), 1.34 (s, 9H).

### Example 3 Synthesis of Complex 9

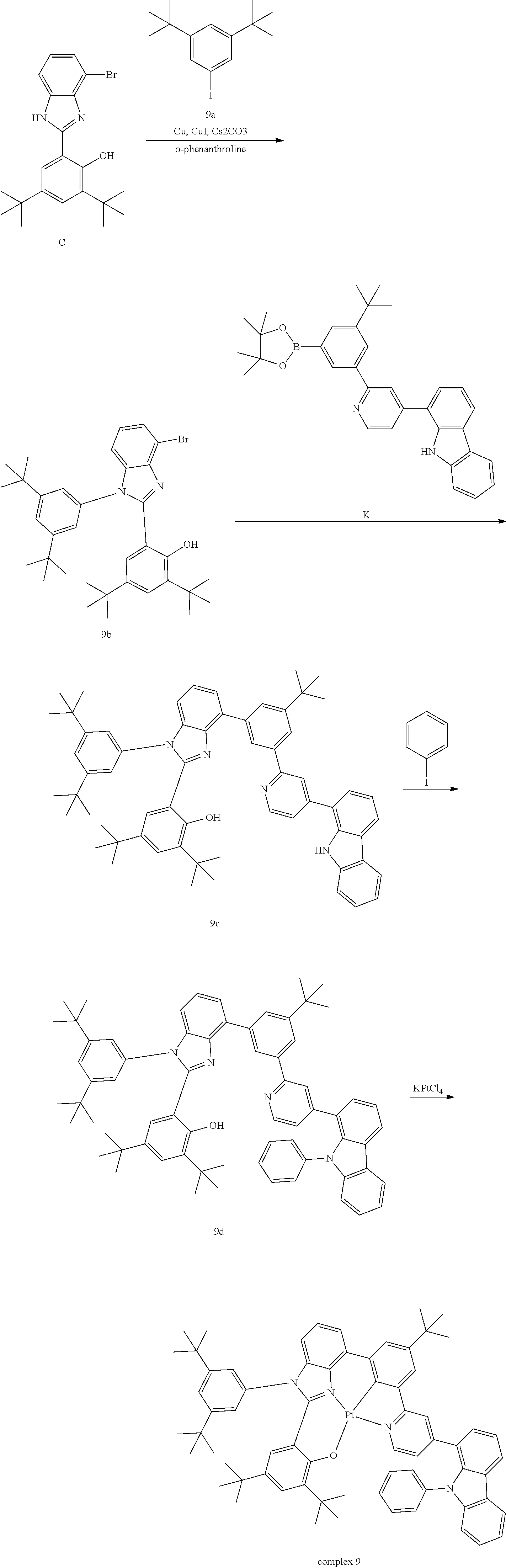

### Synthesis of Compound 9b

The compound C (5.0 g, 12.4 mmol, 1.0 eq), the compound 9a (11.81 g, 37.3 mmol, 3.0 eq), Cu (393 mg, 6.2 mmol, 0.5 eq), CuI (1.18 g, 0.5 eq), 1,10-phenanthroline (2.23 g, 12.4 mmol 1.0 eq), and cesium carbonate (12.1 g, 37.3 mmol, 3.0 eq) were added into a 250 ml three-necked flask to carry out a reaction in an oil bath at a temperature of 160° C. for 72 h under protection of nitrogen gas, with 100 ml anhydrous xylene as a reaction solvent. The reaction liquid was cooled to room temperature, directly suction-filtered with EA as a drip-washing agent to remove the inorganic salt, and subjected to the silica gel column chromatography (with Hex:EA=8:1 as an eluent) to collect and obtain 3.2 g of yellow fluorescent product point with a yield of 43.8%. The H-NMR data is as follows: ¹H NMR (400 MHz, Chloroform-d) δ 7.71 (s, 1H), 7.69 (s, 1H), 7.66 (d, J=2.0 Hz, 1H), 7.59 (s, 1H), 7.43 (t, J=2.0 Hz, 1H), 7.18 (d, J=2.0 Hz, 2H), 7.04 (d, J=2.0 Hz, 1H), 1.42 (s, 9H), 1.36 (d, J=1.5 Hz, 27H).

### Synthesis of Compound 9c

The compound 9b (3.0 g, 5.08 mmol, 1.0 eq), the compound K (2.68 g, 5.34 mmol, 1.05 eq), Pd₁₃₂ (71 mg, 0.1 mmol, 0.02 eq), K₂CO₃ (1.4 g, 10.16 mmol, 2.0 eq), and THF/water (80 ml/16 ml) were added into a 250 ml single-necked flask to carry out a reaction at 70° C. for 12 h under protection of nitrogen gas. After the reaction was finished, the reaction liquid was rotary-evaporated to remove most of the solvent, added with water, extracted with EA twice, mixed with silica gel and rotary-evaporated, and eluted in a silica gel column with Hex:EA=6:1 to obtain 3.6 g of white solid with a yield of 80.2%. The H-NMR data is as follows: ¹H NMR (400 MHz, Chloroform-d) δ 8.58 (d, J=4.9 Hz, 1H), 8.15 (d, J=12.7 Hz, 2H), 7.98 (t, J=2.0 Hz, 1H), 7.88-7.77 (m, 3H), 7.72-7.64 (m, 2H), 7.61 (t, J=2.0 Hz, 1H), 7.54 (d, J=5.5 Hz, 2H), 7.50 (s, 1H), 7.43 (t, J=2.0 Hz, 1H), 7.38 (dd, J=5.0, 1.0 Hz, 1H), 7.31 (t, J=1.9 Hz, 1H), 7.28-7.16 (m, 4H), 7.04 (d, J=2.0 Hz, 1H), 1.42 (s, 9H), 1.36 (d, J=1.4 Hz, 36H).

### Synthesis of Compound 9d

The compound 9c (3.5 g, 3.95 mmol, 1.0 eq), iodobenzene (2.42 g, 11.8 mmol, 3.0 eq), Cu (125 mg, 1.97 mmol, 0.5 eq), CuI (375 mg, 1.97 mmol, 0.5 eq), 1,10-phenanthroline (783 mg, 3.95 mmol, 1.0 eq), and cesium carbonate (3.86 g, 11.85 mmol, 3.0 eq) were added into a 250 ml three-necked flask to carry out a reaction in an oil bath at a temperature of 160° C. for 72 h under protection of nitrogen gas, with 100 ml anhydrous xylene as a reaction solvent. The reaction liquid was cooled to room temperature, directly suction-filtered with EA as a drip-washing agent to remove the inorganic salt, and subjected to the silica gel column chromatography (with Hex:EA=5:1 as an eluent) to collect and obtain 1.66 g of yellow fluorescent product point with a yield of 43.8%. The H-NMR data is as follows: ¹H NMR (400 MHz, Chloroform-d) δ 7.71 (s, 1H), 7.69 (s, 1H), 7.66 (d, J=2.0 Hz, 1H), 7.59 (s, 1H), 7.43 (t, J=2.0 Hz, 1H), 7.18 (d, J=2.0 Hz, 2H), 7.04 (d, J=2.0 Hz, 1H), 1.42 (s, 9H), 1.36 (d, J=1.5 Hz, 27H).

### Synthesis of Complex 9

The compound 9d (1.44 g, 1.5 mmol, 1.0 eq.), KPtCl₄ (676 mg, 1.8 mmol, 1.2 eq.), TBAB (72.4 mg, 0.225 mmol, 0.15 eq.), and 80 mL acetic acid were added to carry out a reaction at 130° C. for 48 h under protection of argon gas. After the reaction was finished and the reaction liquid was cooled, the reaction liquid was added into 400 mL deionized water and stirred for 10 min to precipitate a large amount of solid. The solid was suction-filtered out, dissolved with dichloromethane, separated by silica gel column twice (pure dichloromethane was used in the first time, and n-hexane:dichloromethane=2/1 (V/V/) was used in the second time) to obtain 1.45 g of product. The obtained product was triturated with 20 mL Hex to obtain 1.05 g final product with a yield of 61.1% and a purity of 99.90%. The H-NMR data is as follows: ¹H NMR (400 MHz, Chloroform-d) δ 8.20 (s, 1H), 8.13 (s, 1H), 8.06 (d, J=1.4 Hz, 2H), 7.87 (s, 1H), 7.77 (d, J=15.9 Hz, 2H), 7.69 (d, J=12.6 Hz, 2H), 7.62 (s, 1H), 7.57 (s, 1H), 7.52-7.44 (m, 4H), 7.43-7.37 (m, 5H), 7.35 (s, 1H), 7.26 (s, 1H), 7.23 (d, J=2.0 Hz, 2H), 7.05 (d, J=2.0 Hz, 1H), 6.97 (s, 1H), 6.66 (s, 1H), 1.38 (s, 9H), 1.36 (d, J=1.4 Hz, 36H).

### Example 4: Synthesis of Complex 25

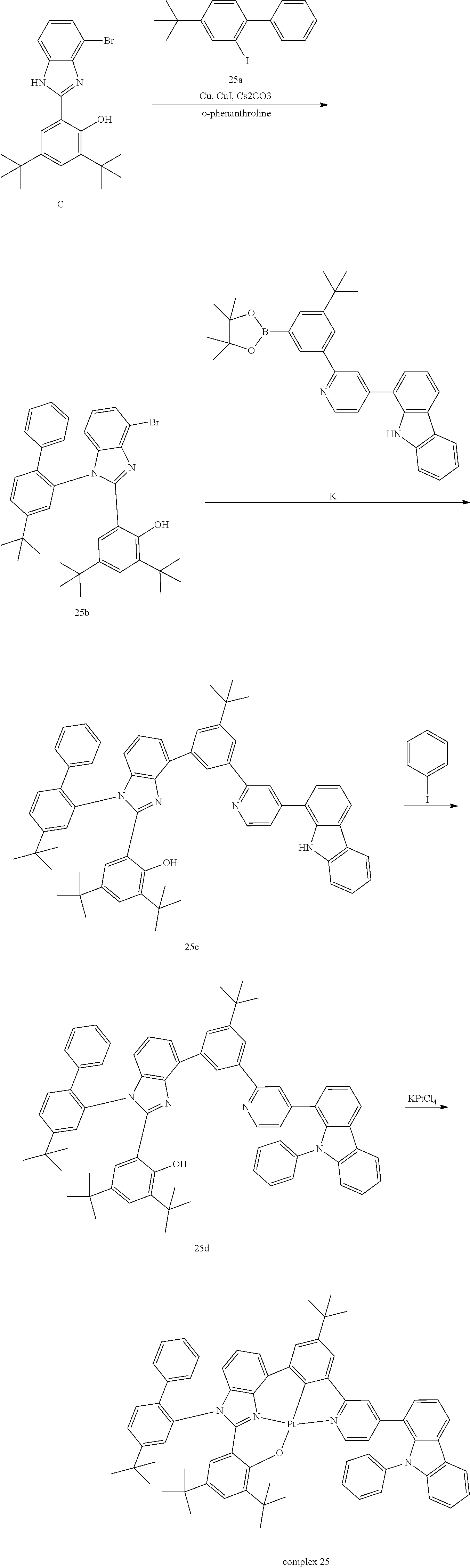

### Synthesis of Compound 25b

The compound C (5.0 g, 12.4 mmol, 1.0 eq), the compound 25a (12.54 g, 37.3 mmol, 3.0 eq), Cu (393 mg, 6.2 mmol, 0.5 eq), CuI (1.18 g, 0.5 eq), 1,10-phenanthroline (2.23 g, 12.4 mmol, 1.0 eq), and cesium carbonate (12.1 g, 37.3 mmol, 3.0 eq) were added into a 250 ml three-necked flask to carry out a reaction in an oil bath at a temperature of 160° C. for 72 h under protection of nitrogen gas, with 100 ml anhydrous xylene as a reaction solvent. The reaction liquid was cooled to room temperature, directly suction-filtered with EA as a drip-washing agent to remove the inorganic salt, and subjected to the silica gel column chromatography (with Hex:EA=8:1 as an eluent) to collect and obtain 3.5 g of yellow fluorescent product point with a yield of 46.3%. The H-NMR data is as follows: ¹H NMR (400 MHz, Chloroform-d) δ 7.70 (d, J=7.1 Hz, 2H), 7.66 (d, J=2.0 Hz, 1H), 7.59 (s, 1H), 7.53 (d, J=7.5 Hz, 1H), 7.50-7.46 (m, 2H), 7.45-7.34 (m, 3H), 7.22 (dd, J=7.5, 2.0 Hz, 1H), 7.12 (d, J=2.0 Hz, 1H), 7.04 (d, J=2.0 Hz, 1H). 1.42 (s, 9H), 1.35 (d, J=7.7 Hz, 18H).

### Synthesis of Compound 25c

The compound 25b (3.3 g, 5.41 mmol, 1.0 eq), the compound K (2.86 g, 5.68 mmol, 1.05 eq), Pd₁₃₂ (78.1 mg, 0.11 mmol, 0.02 eq), K₂CO₃ (1.5 g, 10.82 mmol, 2.0 eq), and THF/water (80 ml/16 ml) were added into a 250 ml single-necked flask to carry out a reaction at 70° C. for 12 h under protection of nitrogen gas. After the reaction was finished, the reaction liquid was rotary-evaporated to remove most of the solvent, added with water, extracted with EA twice, mixed with silica gel and rotary-evaporated, and eluted in a silica gel column with Hex:EA=6:1 to obtain 3.8 g of white solid with a yield of 77.5%. The H-NMR data is as follows: ¹H NMR (400 MHz, Chloroform-d) δ 8.58 (d, J=4.9 Hz, 1H), 8.15 (d, J=12.7 Hz, 2H), 7.98 (t, J=2.0 Hz, 1H), 7.89-7.82 (m, 2H), 7.79 (s, 1H), 7.66 (d, J=2.0 Hz, 2H), 7.61 (t, J=2.0 Hz, 1H), 7.56-7.51 (m, 3H), 7.50-7.46 (m, 3H), 7.45-7.34 (m, 4H), 7.31 (t, J=1.9 Hz, 1H), 7.28-7.19 (m, 3H), 7.12 (d, J=2.1 Hz, 1H), 7.04 (d, J=2.0 Hz, 1H), 1.42 (s, 9H), 1.38-1.32 (m, 27H).

### Synthesis of Compound 25d

The compound 25c (3.6 g, 3.97 mmol, 1.0 eq), iodobenzene (2.45 g, 12 mmol, 3.0 eq), Cu (127 mg, 2 mmol, 0.5 eq), CuI (380 mg, 2 mmol, 0.5 eq), 1,10-phenanthroline (793 mg, 4 mmol, 1.0 eq), and cesium carbonate (3.9 g, 12 mmol, 3.0 eq) were added into a 250 ml three-necked flask to carry out a reaction in an oil bath at a temperature of 160° C. for 72 h under protection of nitrogen gas, with 100 ml anhydrous xylene as a reaction solvent. The reaction liquid was cooled to room temperature, directly suction-filtered with EA as a drip-washing agent to remove the inorganic salt, and subjected to the silica gel column chromatography (with Hex:EA=5:1 as an eluent) to collect and obtain 1.98 g of yellow fluorescent product point with a yield of 46.3%. The H-NMR data is as follows: ¹H NMR (400 MHz, Chloroform-d) δ 8.58 (d, J=4.9 Hz, 1H), 8.16 (d, J=31.0 Hz, 2H), 7.98 (t, J=2.0 Hz, 1H), 7.87-7.76 (m, 3H), 7.70-7.64 (m, 3H), 7.61 (t, J=2.0 Hz, 1H), 7.58-7.51 (m, 3H), 7.51-7.44 (m, 4H), 7.43-7.34 (m, 8H), 7.31 (t, J=1.9 Hz, 1H), 7.28-7.19 (m, 2H), 7.12 (d, J=2.0 Hz, 1H), 7.04 (d, J=2.0 Hz, 1H), 1.42 (s, 9H), 1.38-1.32 (m, 27H).

### Synthesis of Complex 25

The compound 25d (1.47 g, 1.5 mmol, 1.0 eq.), KPtCl₄ (676 mg, 1.8 mmol, 1.2 eq.), TBAB (72.4 mg, 0.225 mmol, 0.15 eq.), and 80 mL acetic acid were added to carry out a reaction at 130° C. for 48 h under protection of argon gas. After the reaction was finished and the reaction liquid was cooled, the reaction liquid was added into 400 mL deionized water and stirred for 10 min to precipitate a large amount of solid. The solid was suction-filtered out, dissolved with dichloromethane, separated by silica gel column twice (pure dichloromethane was used in the first time, and n-hexane:dichloromethane=2/1 (V/V/) was used in the second time) to obtain 1.21 g product. The obtained product was triturated with 20 mL Hex to obtain 0.93 g final product with a yield of 53.1% and a purity of 99.92%. The H-NMR data is as follows: ¹H NMR (400 MHz, Chloroform-d) δ 8.16 (d, J=31.0 Hz, 2H), 8.06 (d, J=1.4 Hz, 2H), 7.87 (s, 1H), 7.79 (s, 1H), 7.73-7.65 (m, 3H), 7.62 (s, 1H), 7.59-7.44 (m, 8H), 7.43-7.34 (m, 8H), 7.29-7.20 (m, 2H), 7.11 (d, J=2.0 Hz, 1H), 7.05 (d, J=2.0 Hz, 1H), 6.97 (s, 1H), 6.67 (s, 1H), 1.38 (s, 9H), 1.37-1.33 (m, 27H).

### Example 5: Synthesis of Complex 125

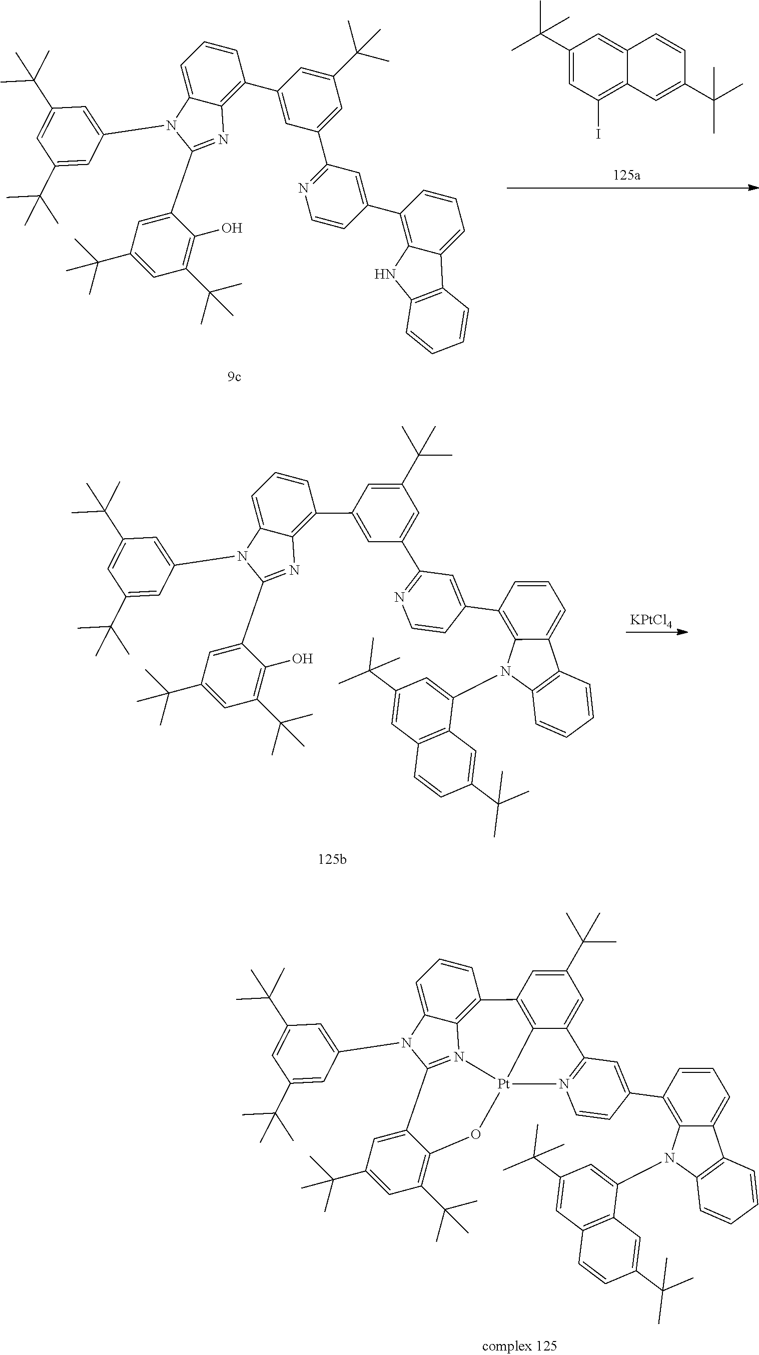

### Synthesis of Compound 125b

The compound 9c (5 g, 5.65 mmol, 1.0 eq), the compound 125a (6.19 g, 16.9 mmol, 3.0 eq), Cu (179.4 mg, 2.82 mmol, 0.5 eq), CuI (535.8 mg, 2.82 mmol, 0.5 eq), 1,10-phenanthroline (1.12 g, 5.65 mmol, 1.0 eq) and cesium carbonate (5.52 g, 16.95 mmol, 3.0 eq) were added into a 500 ml three-necked flask to carry out a reaction in an oil bath at a temperature of 160° C. for 72 h under protection of nitrogen gas, with 180 ml anhydrous xylene as a reaction solvent. The reaction liquid was cooled to room temperature, directly suction-filtered with EA as a drip-washing agent to remove the inorganic salt, and subjected to the silica gel column chromatography (with Hex:EA=5:1 as an eluent) to collect and obtain 3.15 g of yellow fluorescent product point with a yield of 49.6%. The H-NMR data is as follows: ¹H NMR (400 MHz, Chloroform-d) δ 8.58 (d, J=4.9 Hz, 1H), 8.20 (s, 1H), 8.13 (s, 1H), 7.98 (t, J=2.0 Hz, 1H), 7.84 (d, J=1.0 Hz, 1H), 7.79 (s, 2H), 7.75-7.65 (m, 5H), 7.61 (dd, J=4.3, 2.3 Hz, 2H), 7.56 (d, J=9.1 Hz, 2H), 7.45-7.33 (m, 4H), 7.31 (t, J=1.9 Hz, 1H), 7.26 (s, 1H), 7.22-7.16 (m, 3H), 7.04 (d, J=2.0 Hz, 1H), 1.42 (s, 9H), 1.39-1.32 (m, 54H).

### Synthesis of Complex 125

The compound 125b (3 g, 2.6 mmol, 1.0 eq.), KPtCl₄ (1.2 g, 3.2 mmol, 1.2 eq.), TBAB (125.7 mg, 0.39 mmol, 0.15 eq.), and 200 mL acetic acid were added to carry out a reaction at 130° C. for 48 h under protection of argon gas. After the reaction was finished and the reaction liquid was cooled, the reaction liquid was added into 600 mL deionized water and stirred for 10 min to precipitate a large amount of solid. The solid was suction-filtered out, dissolved with dichloromethane, separated by silica gel column twice (pure dichloromethane was used in the first time, and n-hexane:dichloromethane=2/1 (V/V/) was used in the second time) to obtain 2.6 g product. The obtained product was triturated with 20 mL Hex to obtain 1.9 g final product with a yield of 55.6% and a purity of 99.89%. The H-NMR data is as follows: ¹H NMR (400 MHz, Chloroform-d) δ8.20 (s, 1H), 8.13 (s, 1H), 8.06 (d, J=1.4 Hz, 2H), 7.87 (s, 1H), 7.80-7.72 (m, 3H), 7.72-7.66 (m, 3H), 7.62 (s, 2H), 7.57 (s, 1H), 7.49 (d, J=16.5 Hz, 2H), 7.44-7.34 (m, 4H), 7.29-7.18 (m, 4H), 7.05 (d, J=2.0 Hz, 1H), 6.97 (s, 1H), 6.66 (s, 1H), 1.38 (s, 9H), 1.37 (s, 9H), 1.36 (d, J=1.5 Hz, 36H), 1.34 (s, 9H).

### Example 6: Synthesis of Complex 175

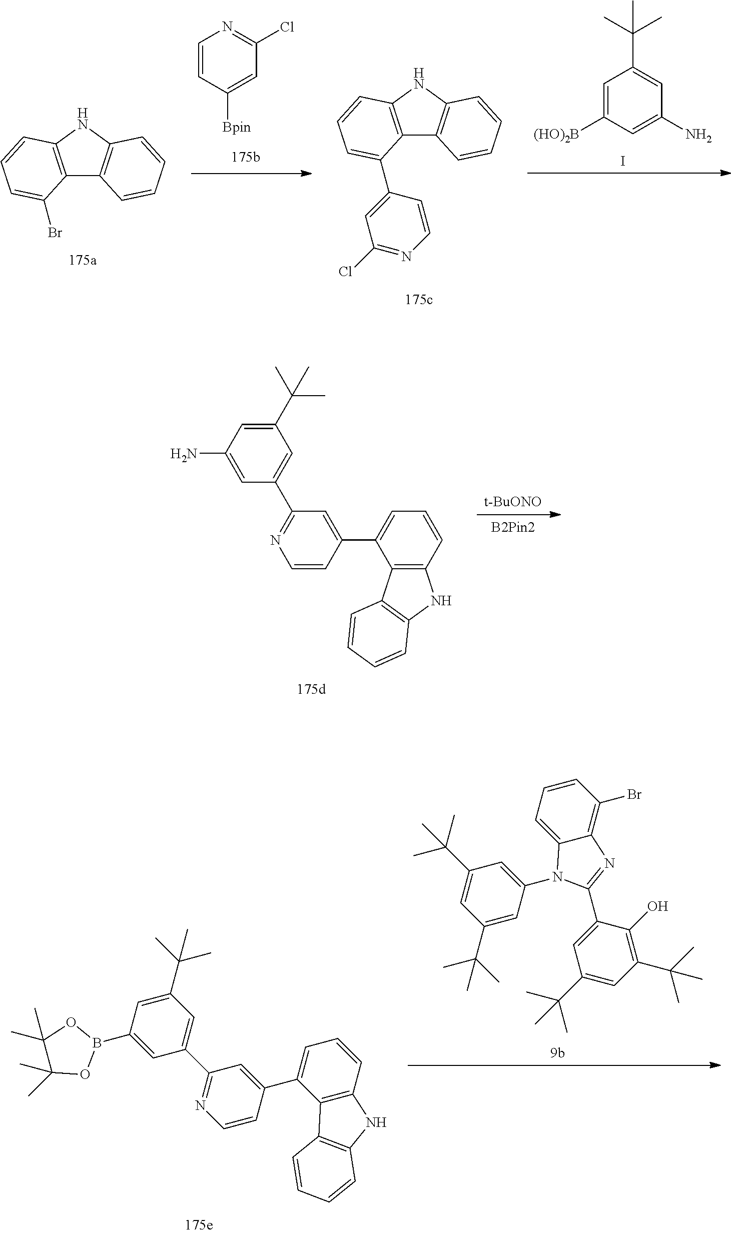

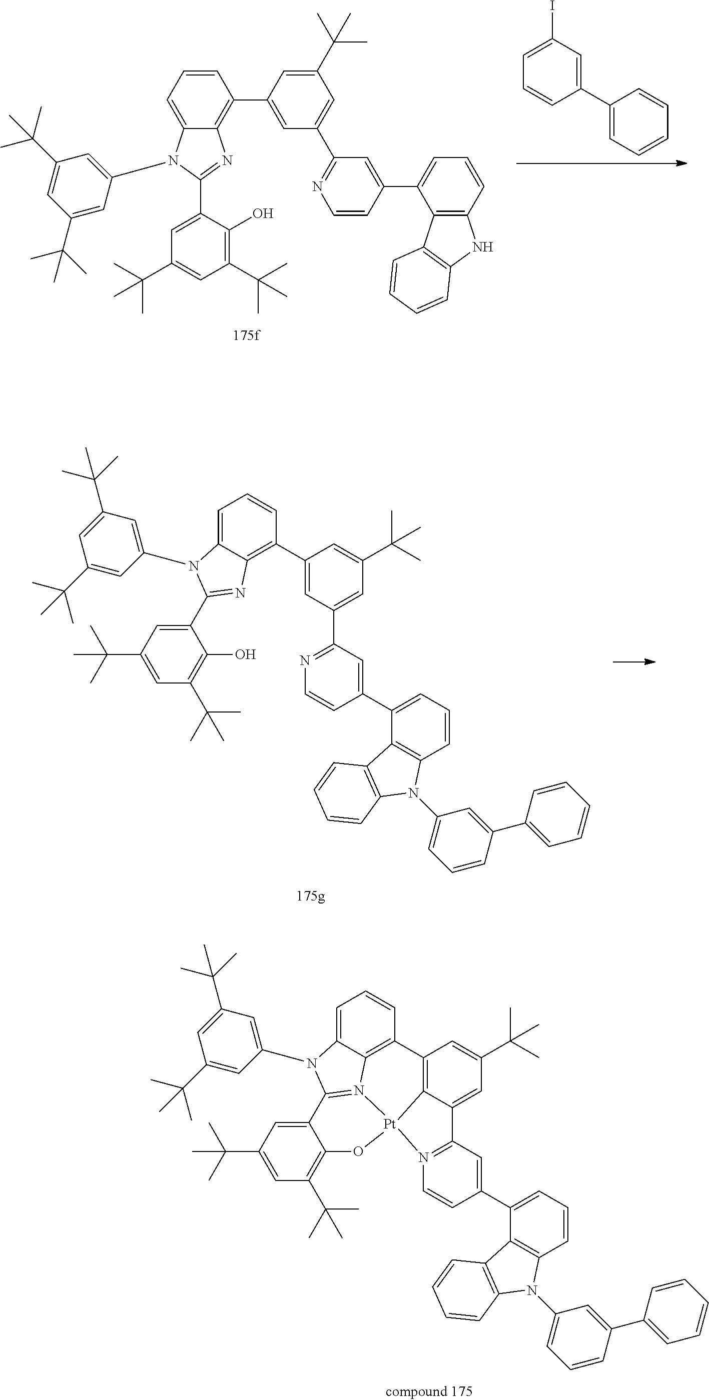

### Synthesis of Complex 175c

The compound 175a (5.0 g, 20.32 mmol, 1.0 eq.), the compound 175b (2.37 g, 21.3 mmol, 1.05 eq.), Pd₁₃₂ (14 mg, 0.02 mmol, 1% eq.), K₂CO₃ (7.01 g, 50.8 mmol, 2.5 eq.), and toluene/ethanol/H₂O (100/100/20 ml) were added into a 500 ml three-necked flask to carry out a reaction at 90° C. for 12 h under stirring and protection of nitrogen gas. After the reaction was finished, the reaction liquid was rotary-evaporated to remove most of the solvent, added with deionized water, extracted with dichloromethane for three times, rotary-evaporated, and separated by silica gel column (Hex:EA=10:1) to obtain 4.82 g of brown solid with a yield of 86.1%. The H-NMR data is as follows: ¹H NMR (400 MHz, CDCl₃) δ 9.67 (s, 1H), 8.36 (d, J=5.0 Hz, 1H), 8.15 (s, 1H), 7.66 (s, 1H), 7.59 (d, J=1.0 Hz, 1H), 7.54 (s, 1H), 7.48 (d, J=7.5 Hz, 3H), 7.35 (dd, J=5.0, 1.0 Hz, 1H), 7.27 (d, J=7.5 Hz, 3H).

### Synthesis of Compound 175d

The compound 175c (4.8 g, 17.2 mmol, 1.0 eq.), the compound I, (4.1 g, 21.5 mmol, 1.25 eq.), Pd₁₃₂ (12.1 mg, 0.017 mmol, 1% eq.), K₂CO₃ (5.93 g, 43 mmol, 2.5 eq.), and toluene/ethanol/H₂O (100/100/20 ml) were added into a 500 ml single-necked flask to carry out a reaction at 90° C. for 12 h under stirring and protection of nitrogen gas. After the reaction was finished, the reaction liquid was rotary-evaporated to remove most of the solvent, added with deionized water, extracted with dichloromethane for three times, rotary-evaporated, and separated by silica gel column (Hex:EA=10:1) to obtain 5.9 g of brown solid with a yield of 88.05%. The H-NMR data is as follows: ¹H NMR (400 MHz, CDCl₃) δ9.67 (s, 1H), 8.59 (d, J=5.0 Hz, 1H), 8.15 (s, 1H), 7.81 (d, J=1.0 Hz, 1H), 7.69-7.64 (m, 2H), 7.54 (s, 1H), 7.48 (d, J=7.5 Hz, 2H), 7.38 (dd, J=5.0, 1.0 Hz, 1H), 7.27 (d, J=7.5 Hz, 2H), 7.20 (t, J=2.0 Hz, 1H), 6.61 (t, J=2.0 Hz, 1H), 4.29 (s, 2H), 1.36 (s, 9H).

### Synthesis of Compound 175e

Tert-butyl nitrite (1.56 g, 10 mmol) was added dropwise to a mixture of bis(pinacolato)diboron (1.3 g, 5 mmol), the compound 175d (1.96 g, 5 mmol), and eosin Y (0.1 mmol) in acetonitrile (50 mL). The resulting mixture was stirred at room temperature under blue LED irradiation for 3 hours (TLC). The mixture diluted with ethyl acetate (50 mL) was filtered through diatomaceous earth and the filtrate was extracted with ethyl acetate (3×20 mL). The extract was washed with brine, dried over anhydrous Na₂SO₄, and evaporated to obtain a crude product. The crude product was purified by silica gel column chromatography (Hex:EA=10:1) to obtain 1.9 g of brown solid with a yield of 76.4%. The H-NMR data is as follows: ¹H NMR (400 MHz, CDCl₃) δ 9.67 (s, 1H), 8.59 (d, J=5.0 Hz, 1H), 8.15 (s, 1H), 7.80 (d, J=1.1 Hz, 1H), 7.69-7.65 (m, 2H), 7.54 (s, 1H), 7.51-7.42 (m, 4H), 7.38 (dd, J=5.0, 1.0 Hz, 1H), 7.27 (d, J=7.5 Hz, 2H), 1.38 (s, 12H), 1.34 (s, 9H).

### Synthesis of Compound 175f

The compound 175e (3.8 g, 7.56 mmol, 1.0 eq), the compound 9b (4.68 g, 7.94 mmol, 1.05 eq), Pd₁₃₂ (107.3 mg, 0.15 mmol, 0.02 eq), K₂CO₃ (2 g, 14.52 mmol, 2.0 eq), and THF/water (100 ml/20 ml) were added into a 250 ml single-necked flask to carry out a reaction at 70° C. for 12 h under protection of nitrogen gas. After the reaction was finished, the reaction liquid was rotary-evaporated to remove most of the solvent, added with water, extracted with EA twice, mixed with silica gel and rotary-evaporated, and eluted in a silica gel column with Hex:EA=6:1 to obtain 5.1 g of white solid with a yield of 76.2%. The H-NMR data is as follows: ¹H NMR (400 MHz, Chloroform-d) δ 9.67 (s, 1H), 8.59 (d, J=5.0 Hz, 1H), 8.15 (s, 1H), 7.98 (t, J=2.0 Hz, 1H), 7.83-7.77 (m, 2H), 7.71-7.64 (m, 3H), 7.61 (t, J=2.0 Hz, 1H), 7.54 (d, J=5.5 Hz, 2H), 7.48 (d, J=7.5 Hz, 2H), 7.43 (t, J=2.0 Hz, 1H), 7.38 (dd, J=5.0, 1.0 Hz, 1H), 7.32-7.25 (m, 3H), 7.18 (d, J=2.0 Hz, 2H), 7.04 (d, J=2.0 Hz, 1H), 1.42 (s, 9H), 1.36 (d, J=1.4 Hz, 36H).

### Synthesis of Compound 175g

The compound 175f (5 g, 5.65 mmol, 1.0 eq), 3-iodobiphenyl (4.75 g, 16.9 mmol, 3.0 eq), Cu (179.4 mg, 2.83 mmol, 0.5 eq), CuI (538 mg, 2.83 mmol, 0.5 eq), 1,10-phenanthroline (1.12 g, 5.65 mmol, 1.0 eq), and cesium carbonate (5.51 g, 16.95 mmol, 3.0 eq) were added into a 250 ml three-necked flask to carry out a reaction in an oil bath at a temperature of 160° C. for 72 h under protection of nitrogen gas, with 150 ml anhydrous xylene as a reaction solvent. The reaction liquid was cooled to room temperature, directly suction-filtered with EA as a drip-washing agent to remove the inorganic salt, and subjected to the silica gel column chromatography (with Hex:EA=5:1 as an eluent) to collect and obtain 3.1 g of yellow fluorescent product point with a yield of 52.9%. The H-NMR data is as follows: ¹H NMR (400 MHz, Chloroform-d) δ 8.59 (d, J=5.0 Hz, 1H), 8.16 (s, 1H), 7.98 (t, J=2.0 Hz, 1H), 7.83-7.77 (m, 3H), 7.73-7.64 (m, 5H), 7.63-7.57 (m, 3H), 7.55 (s, 1H), 7.48-7.30 (m, 12H), 7.27 (s, 1H), 7.18 (d, J=2.0 Hz, 2H), 7.04 (d, J=2.0 Hz, 1H), 1.42 (s, 9H), 1.36 (d, J=1.4 Hz, 36H).

### Synthesis of Complex 175

The compound 175 g (3 g, 2.9 mmol, 1.0 eq.), KPtCl₄ (1.3 g, 3.48 mmol, 1.2 eq.), TBAB (140.2 mg, 0.435 mmol, 0.15 eq.), and 300 ml acetic acid were added to carry out a reaction at 130° C. for 48 h under protection of argon gas. After the reaction was finished and the reaction liquid was cooled, the reaction liquid was added into 600 mL deionized water and stirred for 10 min to precipitate a large amount of solid. The solid was suction-filtered out, dissolved with dichloromethane, separated by silica gel column twice (pure dichloromethane was used in the first time, and n-hexane:dichloromethane=2/1 (V/V/) was used in the second time) to obtain 3.1 g product. The obtained product was triturated with 80 mL Hex to obtain 2.14 g final product with a yield of 61.1% and a purity of 99.90%. The H-NMR data is as follows: ¹H NMR (400 MHz, Chloroform-d) δ 8.16 (s, 1H), 8.06 (s, 1H), 7.96 (s, 1H), 7.87 (s, 1H), 7.79 (t, J=2.0 Hz, 1H), 7.75 (s, 1H), 7.71 (s, 1H), 7.66 (d, J=1.9 Hz, 3H), 7.64-7.57 (m, 3H), 7.50 (s, 1H), 7.48-7.34 (m, 8H), 7.32 (dt, J=7.5, 2.1 Hz, 1H), 7.27 (d, J=5.5 Hz, 2H), 7.23 (d, J=2.0 Hz, 2H), 7.05 (d, J=2.0 Hz, 1H), 6.98 (s, 1H), 1.38 (s, 9H), 1.36 (d, J=1.4 Hz, 36H).

### Example 7: Synthesis of Complex 190

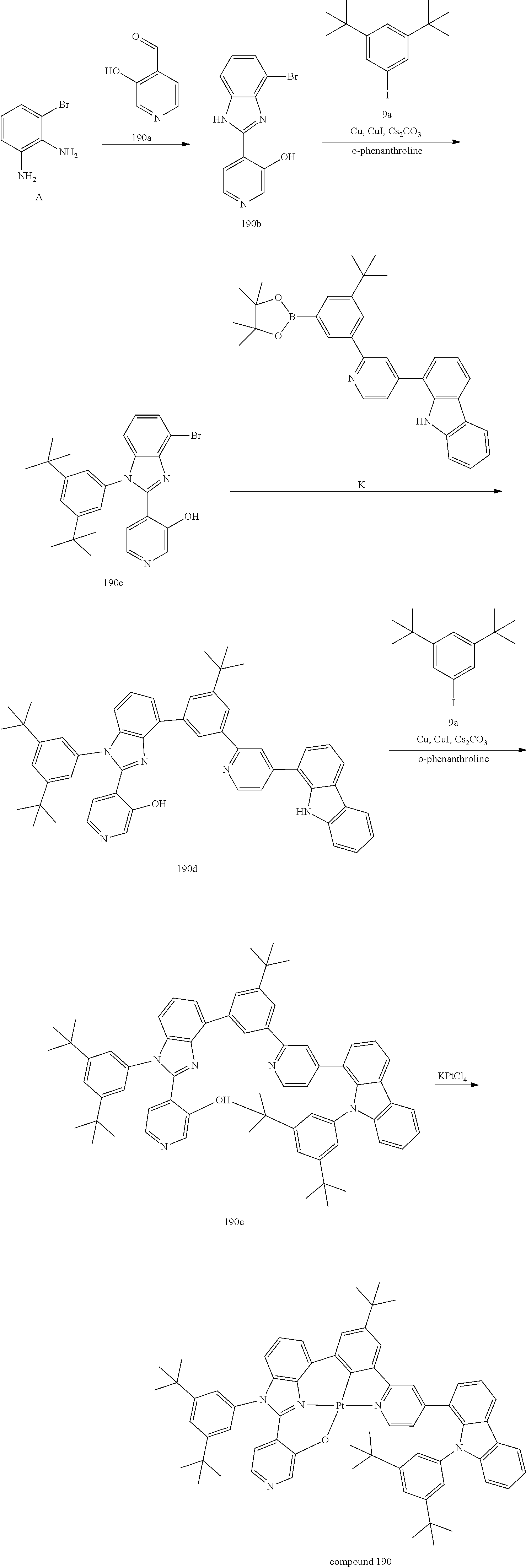

### Synthesis of Compound 190b

The compound A (10.0 g, 53 mmol, 1.0 eq), the compound 190a (6.6 g, 53 mmol, 1.0 eq), Na₂S₂O₅ (508 mg, 2.6 mmol, 0.05 eq), and DMF (200 mL) were added into a 250 mL single-necked flask with N₂ introduced to carry out a reaction at 80° C. for 4 h. After the reaction was finished, the reaction liquid was spotted on a chromatoplate with HEX/EA=10/1 (V/V) as a developing solvent, and the target compound was found, suggesting that the reaction was complete and the reaction liquid was ready for post-processing. The reaction liquid was extracted with water (100 mL)+EA (30 mL) to achieve a phase separation to obtain an EA layer. The EA layer was concentrated and separated by silica gel column (Hex/EA=10/1 (V/V)) to obtain 3.3 g of yellow solid with a yield of 21.3% and a HPLC purity of 99.52%. The H-NMR data is as follows: ¹H NMR (400 MHz, CDCl₃) δ 8.45 (d, J=5.1 Hz, 3H), 7.93 (d, J=5.0 Hz, 1H), 7.64 (s, 1H), 7.55 (s, 1H), 7.47 (s, 1H).

### Synthesis of Compound 190c

The compound 190b (5.0 g, 17.2 mmol, 1.0 eq), the compound 9a (16.32 g, 51.6 mmol, 3.0 eq), Cu (546 mg, 8.6 mmol, 0.5 eq), CuI (1.64 g, 8.6 mmol, 0.5 eq), 1,10-phenanthroline (3.09 g, 17.2 mmol, 1.0 eq), and cesium carbonate (16.8 g, 51.6 mmol, 3.0 eq) were added into a 250 ml three-necked flask to carry out a reaction in an oil bath at a temperature of 160° C. for 72 h under protection of nitrogen gas, with 100 ml anhydrous xylene as a reaction solvent. The reaction liquid was cooled to room temperature, directly suction-filtered with EA as a drip-washing agent to remove the inorganic salt, and subjected to the silica gel column chromatography (with Hex:EA=8:1 as an eluent) to collect and obtain 3.8 g of yellow fluorescent product point with a yield of 46.1%. The H-NMR data is as follows: ¹H NMR (400 MHz, Chloroform-d) δ 8.44 (d, J=4.1 Hz, 2H), 7.82 (d, J=5.0 Hz, 1H), 7.70 (d, J=9.5 Hz, 2H), 7.59 (s, 1H), 7.43 (t, J=2.0 Hz, 1H), 7.18 (d, J=2.0 Hz, 2H), 1.36 (s, 18H).

### Synthesis of Compound 190d

The compound 190c (3.5 g, 7.32 mmol, 1.0 eq), the compound K (3.86 g, 7.69 mmol, 1.05 eq), Pd₁₃₂ (106.5 mg, 0.15 mmol, 0.02 eq), K₂CO₃ (2.0 g, 14.64 mmol, 2.0 eq), and THF/water (120 ml/30 ml) were added into a 250 ml single-necked flask to carry out a reaction at 70° C. for 12 h under protection of nitrogen gas. After the reaction was finished, the reaction liquid was rotary-evaporated to remove most of the solvent, added with water, extracted with EA twice, mixed with silica gel and rotary-evaporated, and eluted in a silica gel column with Hex:EA=6:1 to obtain 4.6 g of white solid with a yield of 81.4%. The H-NMR data is as follows: ¹H NMR (400 MHz, Chloroform-d) δ8.58 (d, J=4.9 Hz, 1H), 8.48-8.43 (m, 2H), 8.15 (d, J=12.7 Hz, 2H), 7.98 (t, J=2.0 Hz, 1H), 7.89-7.77 (m, 4H), 7.70 (s, 1H), 7.61 (t, J=2.0 Hz, 1H), 7.54 (d, J=5.5 Hz, 2H), 7.50 (s, 1H), 7.43 (t, J=2.0 Hz, 1H), 7.38 (dd, J=5.0, 1.0 Hz, 1H), 7.31 (t, J=1.9 Hz, 1H), 7.25 (d, J=13.0 Hz, 2H), 7.18 (d, J=2.0 Hz, 2H), 1.36 (s, 27H).

### Synthesis of Compound 190e

The compound 190d (3.0 g, 3.90 mmol, 1.0 eq), the compound 9a (3.7 g, 11.7 mmol, 3.0 eq), Cu (124 mg, 1.95 mmol, 0.5 eq), CuI (371 mg, 1.95 mmol, 0.5 eq), 1,10-phenanthroline (773 mg, 3.90 mmol, 1.0 eq), and cesium carbonate (3.81 g, 11.7 mmol, 3.0 eq) were added into a 250 ml three-necked flask to carry out a reaction in an oil bath at a temperature of 160° C. for 72 h under protection of nitrogen gas, with 100 ml anhydrous xylene as a reaction solvent. The reaction liquid was cooled to room temperature, directly suction-filtered with EA as a drip-washing agent to remove the inorganic salt, and subjected to the silica gel column chromatography (with Hex:EA=5:1 as an eluent) to collect and obtain 2.72 g of yellow fluorescent product point with a yield of 72.4%. The H-NMR data is as follows: ¹H NMR (400 MHz, Chloroform-d) δ 8.58 (d, J=4.9 Hz, 1H), 8.48-8.43 (m, 2H), 8.20 (s, 1H), 8.13 (s, 1H), 7.98 (t, J=2.0 Hz, 1H), 7.86-7.77 (m, 4H), 7.69 (d, J=8.1 Hz, 2H), 7.61 (t, J=2.0 Hz, 1H), 7.56 (d, J=9.2 Hz, 2H), 7.43 (t, J=1.9 Hz, 2H), 7.40 (dd, J=5.0, 1.0 Hz, 1H), 7.35 (s, 1H), 7.31 (t, J=1.9 Hz, 1H), 7.26 (s, 1H), 7.20 (dd, J=15.2, 2.0 Hz, 4H), 1.36 (s, 45H).

### Synthesis of Complex 190

The compound 190e (2.5 g, 2.6 mmol, 1.0 eq.), KPtCl₄ (1.17 g, 3.12 mmol, 1.2 eq.), TBAB (125.7 mg, 0.39 mmol, 0.15 eq.), and 80 mL acetic acid were added to carry out a reaction at 130° C. for 48 h under protection of argon gas. After the reaction was finished and the reaction liquid was cooled, the reaction liquid was added into 400 mL deionized water and stirred for 10 min to precipitate a large amount of solid. The solid was suction-filtered out, dissolved with dichloromethane, separated by silica gel column twice (pure dichloromethane was used in the first time, and n-hexane:dichloromethane=2/1 (V/V/) was used in the second time) to obtain 1.45 g product. The obtained product was triturated with 20 mL Hex to obtain 1.9 g final product with a yield of 63.2% and a purity of 99.90%. The H-NMR data is as follows: ¹H NMR (400 MHz, Chloroform-d) δ 8.58 (d, J=5.0 Hz, 1H), 8.27 (s, 1H), 8.20 (s, 1H), 8.13 (s, 1H), 8.06 (d, J=1.4 Hz, 2H), 7.89-7.83 (m, 2H), 7.78 (d, J=8.2 Hz, 2H), 7.69 (d, J=12.6 Hz, 2H), 7.62 (s, 1H), 7.57 (s, 1H), 7.49 (d, J=16.5 Hz, 2H), 7.43 (t, J=1.9 Hz, 2H), 7.35 (s, 1H), 7.28-7.19 (m, 5H), 7.02 (s, 1H), 6.90 (s, 1H), 1.36 (s, 45H).

Those skilled in the art should be understood that the above preparation methods are only several exemplary examples, and modifications can be made on them by those skilled in the art to obtain the structures of other compounds in the present application.

### Example 8

Under an atmosphere nitrogen gas, fully dried samples of platinum complexes 9, 25, 125, 17 and 190 were each weighted at approximately 5.0 mg and subjected to a heating scan at a rate of 10° C./min within a range of 25-800° C. The determined thermal decomposition temperatures (corresponding to a thermal weight loss of 0.5%) were 421° C., 449° C., 434° C., 412° C., and 424° C., respectively, suggesting the excellent thermal stability of these complexes.

### Example 9

An organic light-emitting diode was prepared using the complex luminescent material of the present application, and the structure of the device is as shown in the FIGURE.

Firstly, a transparent conductive ITO glass substrate 10 (provided with an anode 20 thereon) was washed sequentially with a detergent solution and deionized water, ethanol, acetone, and deionized water, and then treated with oxygen plasma for 30 seconds.

Then, HATCN was evaporated onto ITO as a hole injection layer 30 with a thickness of 10 nm.

Then, compound HT was evaporated to form a hole transport layer 40 with a thickness of 40 nm.

Then, a light-emitting layer 50 with a thickness of 20 nm was evaporated onto the hole transport layer. The light-emitting layer was formed of the platinum complex 9 (20%) in combination with CBP (80%).

Then, AlQ₃ was evaporated onto the light-emitting layer as an electron transport layer 60 with a thickness of 40 nm.

Finally, 1 nm LiF was evaporated as an electron injection layer 70 and 100 nm Al was evaporated as a cathode 80 of the device.

Example 10: An organic light-emitting diode was prepared by the method as described in Example 9, with the complex 9 replaced by the complex 25.

Example 11: An organic light-emitting diode was prepared by the method as described in Example 9, with the complex 9 replaced by the complex 125.

Example 12: An organic light-emitting diode was prepared by the method as described in Example 9, with the complex 9 replaced by the complex 175.

Example 13: An organic light-emitting diode was prepared by the method as described in Example 9, with the complex 9 replaced by the complex 190.

### Comparative Example 1

An organic light-emitting diode was prepared by the method as described in Example 9, with the complex 9 replaced by the complex Ref-1(CN107573383A).

### Comparative Example 2

An organic light-emitting diode was prepared by the method as described in Example 9, with the complex 9 replaced by the complex Ref-2 (CN107573383A).

HATCN, HT, AlQ₃, Ref-1, Ref-2, and CBP in the device have the following structural formulas:

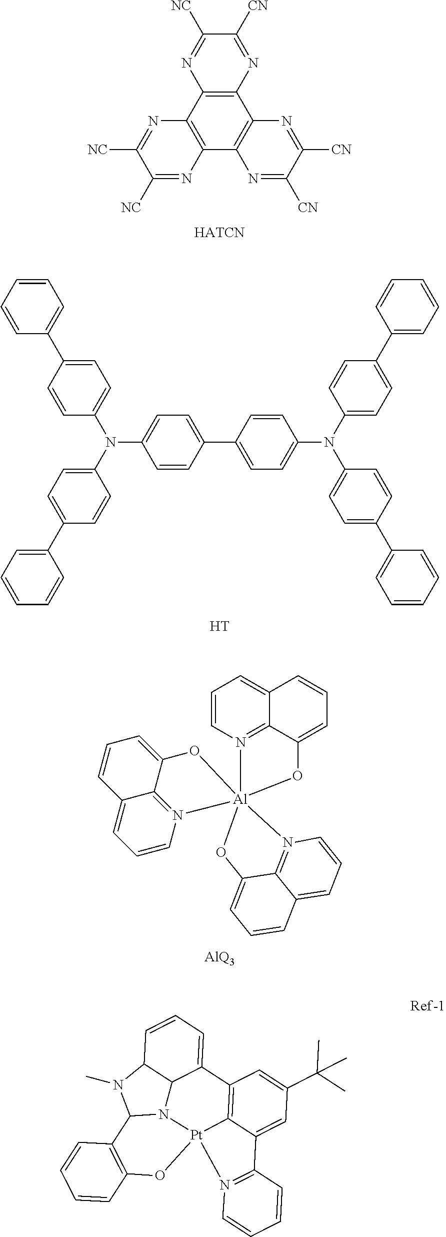

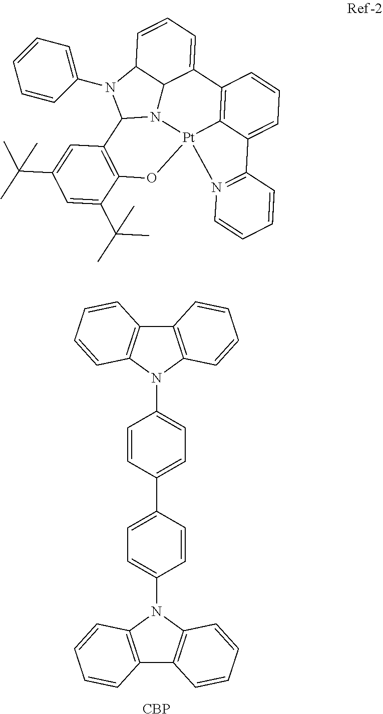

The device performances of the organic electroluminescent devices in Examples 9 to 13, Comparative example 1, and Comparative example 2 at a current density of 20 mA/cm² are listed in Table 1.

TABLE 1
tests of performances of organic electroluminescent devices

| | | Driving | Luminous | Device lifespan |
|---|---|---|---|---|
| Device No. | Complex | voltage | efficiency | (LT95) |
| Example 9 | Complex 9 | 0.8 | 1 | 1 |
| Example 10 | Complex 25 | 0.9 | 1.2 | 0.91 |
| Example 11 | Complex 125 | 0.9 | 1.2 | 1.3 |
| Example 12 | Complex 175 | 0.8 | 1.3 | 0.8 |
| Example 13 | Complex 190 | 0.8 | 1.6 | 0.8 |
| Comparative | Ref-1 | 1.1 | 0.51 | 0.24 |
| example 1 | | | | |
| Comparative | Ref-2 | 1.1 | 0.59 | 0.06 |
| example 2 | | | | |

Note:
The performances of the device were tested with Example 9 as a benchmark, with individual indicators as 1. LT95 refers to a duration until the device's brightness diminishes to 95% of the initial brightness (10000 cd/m²).

From the data shown in Table 1, it can be seen that under the same condition, the platinum complex materials of the present application can be used to prepare the organic light-emitting diodes with lower driving voltages and higher luminous efficiencies. In addition, the device lifespans of the organic light-emitting diode based on the complexes of the present application are significantly longer as compared to the complex materials in the Comparative Examples, meeting the requirements on the luminescent materials in the display industry and having good industrialization prospects.

1. A platinum complex, being a compound having a structure of formula (I): 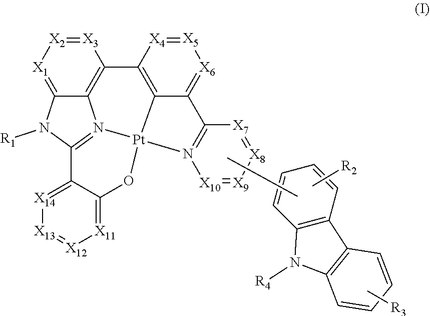
wherein,
X₁ to X₁₄ are each independently selected from the group consisting of N, C, and CR₀;
R₀ to R₄ are each independently selected from the group consisting of hydrogen, deuterium, halogen, amino, carbonyl, carboxyl, thioalkyl, cyano, sulfonyl, phosphino, a substituted or unsubstituted alkyl having 1 to 20 carbon atoms, a substituted or unsubstituted cycloalkyl having 3 to 20 ring carbon atoms, a substituted or unsubstituted alkenyl having 2 to 20 carbon atoms, a substituted or unsubstituted alkoxy having 1 to 20 carbon atoms, a substituted or unsubstituted aryl having 6 to 30 carbon atoms, and a substituted or unsubstituted heteroaryl having 3 to 30 carbon atoms; or adjacent groups of R₀ to R₄ are optionally linked to form a ring;
the “substituted” refers to substitution with halogen, amino, cyano, or C₁-C₄ alkyl;
a heteroatom in the heteroaryl comprises one or more of N, S, or O.

2. The platinum complex of claim 1, wherein R₀ to R₄ are each independently selected from the group consisting of hydrogen, deuterium, halogen, amino, thioalkyl, cyano, a substituted or unsubstituted alkyl having 1 to 6 carbon atoms, a substituted or unsubstituted cycloalkyl having 3 to 6 ring carbon atoms, a substituted or unsubstituted alkenyl having 2 to 6 carbon atoms, a substituted or unsubstituted alkoxy having 1 to 6 carbon atoms, a substituted or unsubstituted aryl having 6 to 12 carbon atoms, and a substituted or unsubstituted heteroaryl having 3 to 6 carbon atoms.

3. The platinum complex of claim 2, wherein R₀, R₂, and R₃ are each independently selected from the group consisting of hydrogen, deuterium, halogen, a C₁-C₄ alkyl, cyano, a substituted or unsubstituted cycloalkyl having 3 to 6 ring carbon atoms, a substituted or unsubstituted aryl having 6 to 12 carbon atoms, and a substituted or unsubstituted heteroaryl having 3 to 6 carbon atoms;
R₁ and R₄ are each independently selected from the group consisting of a substituted or unsubstituted cycloalkyl having 3 to 6 ring carbon atoms, a substituted or unsubstituted aryl having 6 to 12 carbon atoms, and a substituted or unsubstituted heteroaryl having 3 to 6 carbon atoms.

4. The platinum complex of claim 3, wherein R₀, R₂, and R₃ are each independently selected from the group consisting of hydrogen, deuterium, methyl, isopropyl, isobutyl, tert-butyl, cyano, substituted or unsubstituted cyclopentyl, substituted or unsubstituted cyclohexyl, substituted or unsubstituted phenyl, substituted or unsubstituted biphenyl, substituted or unsubstituted naphthyl, substituted or unsubstituted pyridinyl, substituted or unsubstituted pyrazinyl, and substituted or unsubstituted pyrimidinyl;
R₁ and R₄ are each independently selected from the group consisting of substituted or unsubstituted cyclopentyl, substituted or unsubstituted cyclohexyl, substituted or unsubstituted phenyl, substituted or unsubstituted biphenyl, substituted or unsubstituted pyridinyl, substituted or unsubstituted pyrazinyl, substituted or unsubstituted pyrimidinyl, substituted or unsubstituted naphthyl, and substituted or unsubstituted quinolyl.

5. The platinum complex of claim 1, wherein X₁ to X₇ and X₉ to X₁₀ are each independently CR₀, X₈ is C, X₁₁ to X₁₄ are each independently CR₀ or N, and only one of X₁₁ to X₁₄ is N.

6. The platinum complex of claim 5, wherein R₀ is selected from the group consisting of hydrogen, deuterium, methyl, isopropyl, isobutyl, tert-butyl, substituted or unsubstituted phenyl, substituted or unsubstituted biphenyl, and substituted or unsubstituted naphthyl; R₂ and R₃ are each independently selected from the group consisting of hydrogen, deuterium, methyl, isopropyl, isobutyl, and tert-butyl; and R₁ and R₄ are each independently selected from the group consisting of substituted or unsubstituted phenyl, substituted or unsubstituted biphenyl, and substituted or unsubstituted naphthyl.

7. The platinum complex of claim 1, wherein the platinum complex is one of the following compounds:

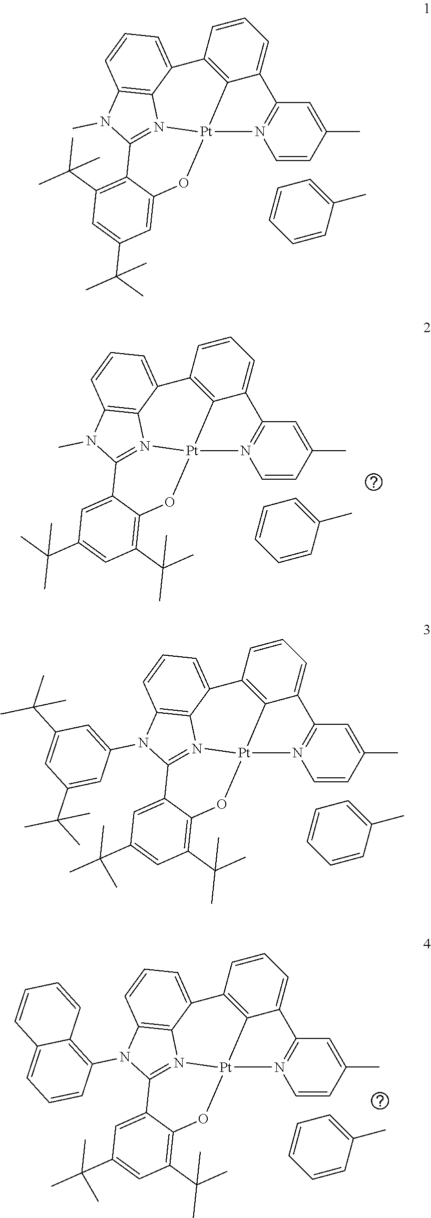

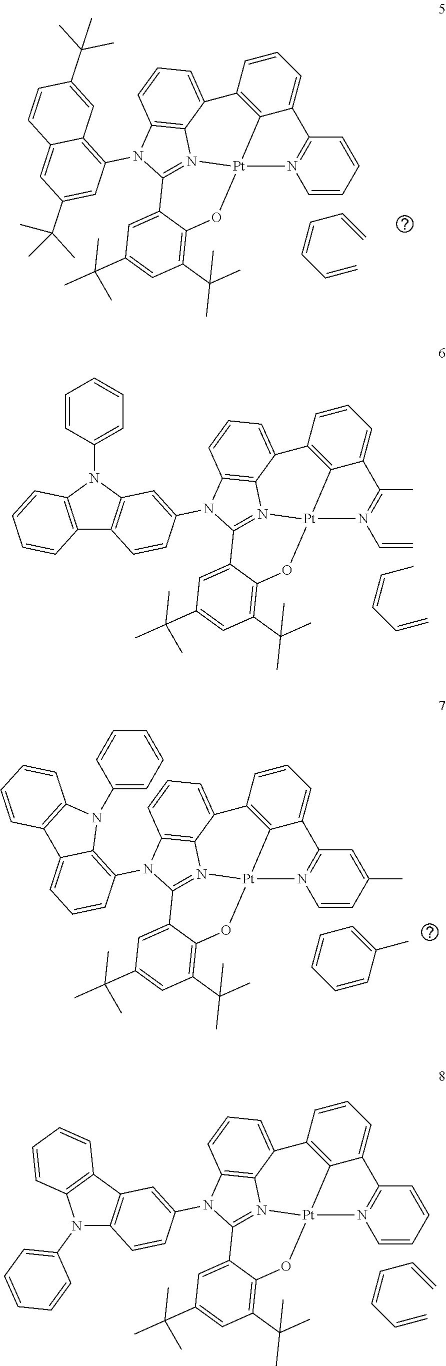

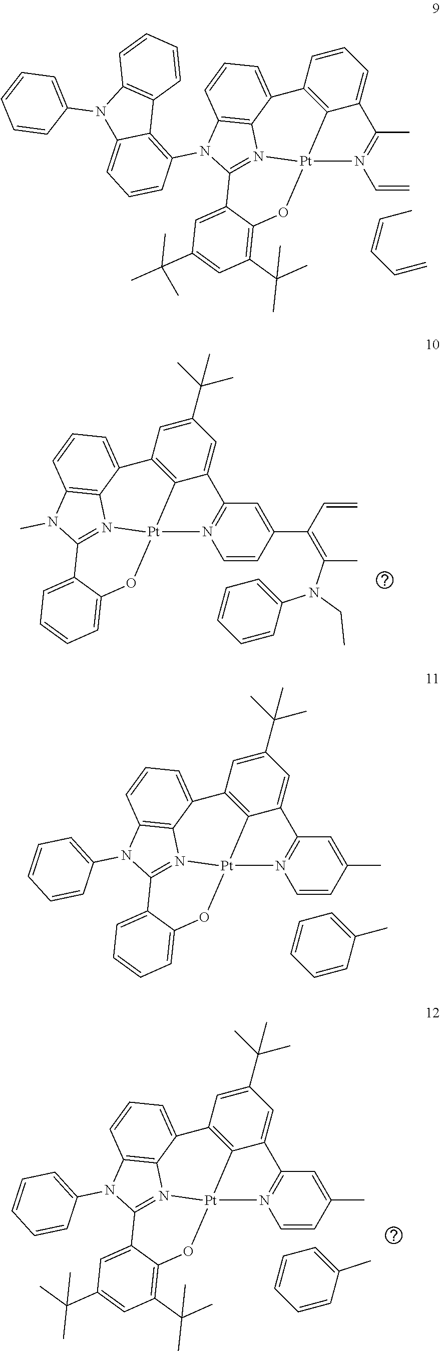

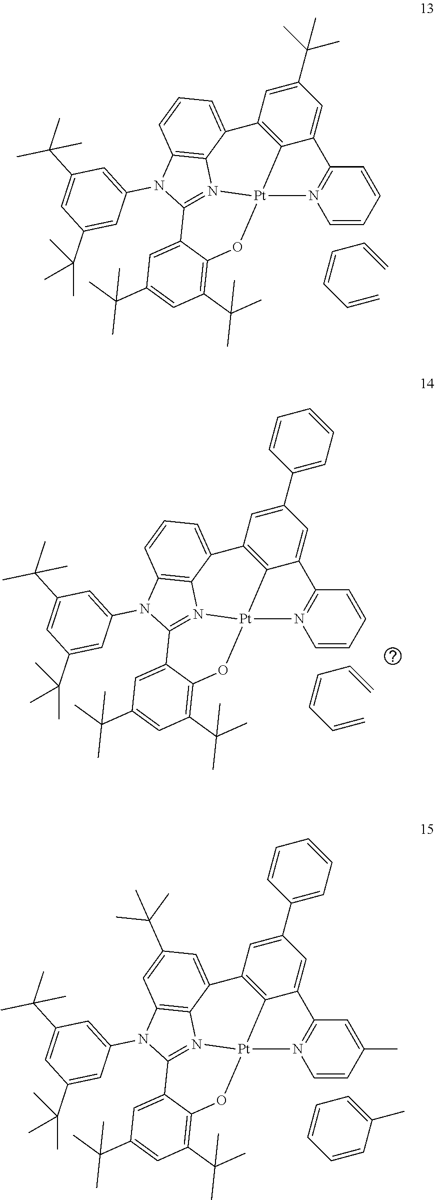

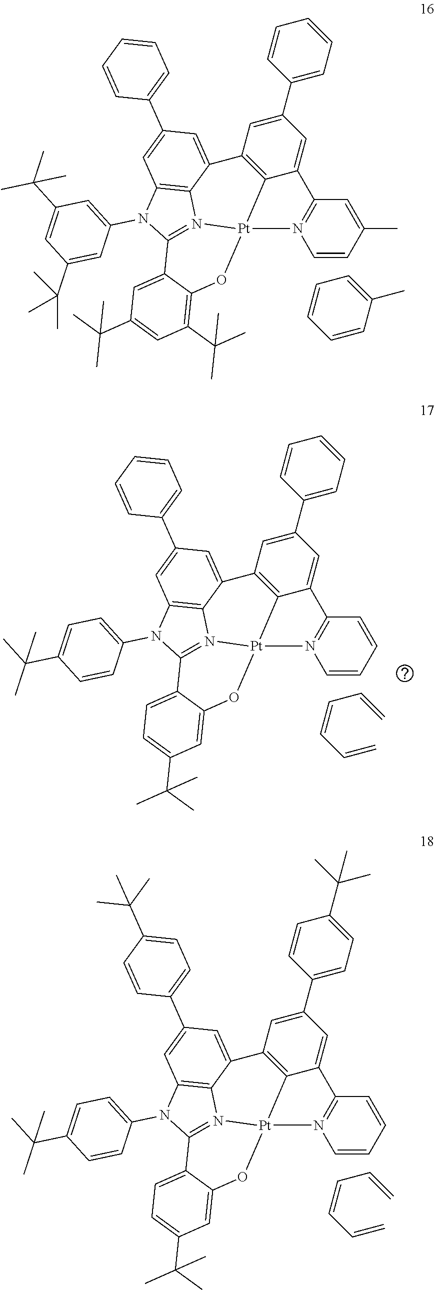

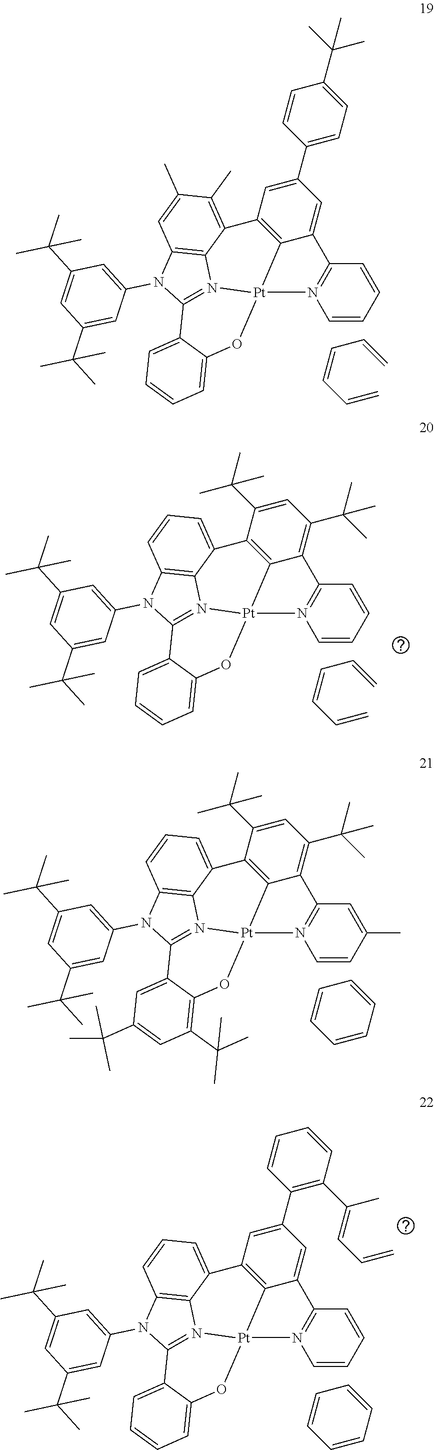

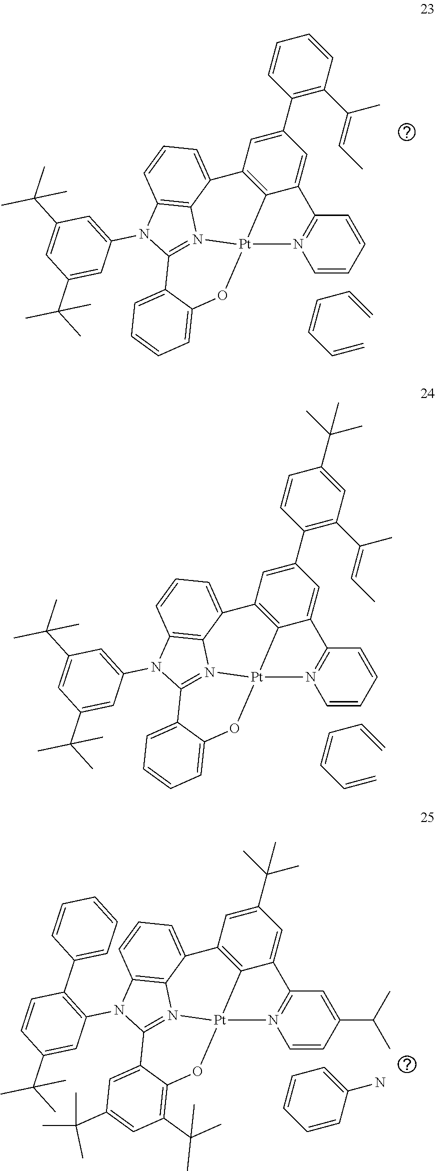

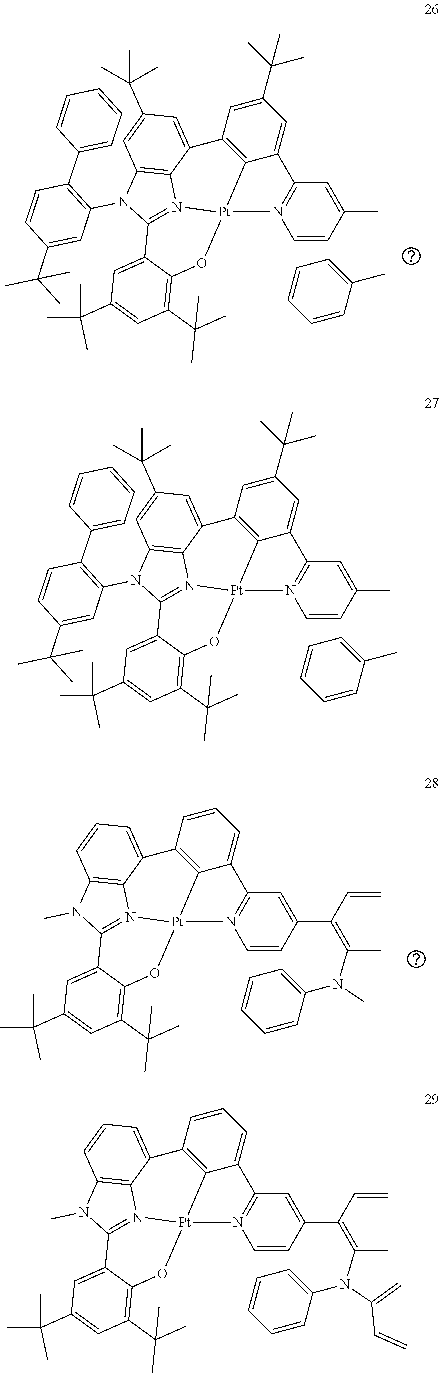

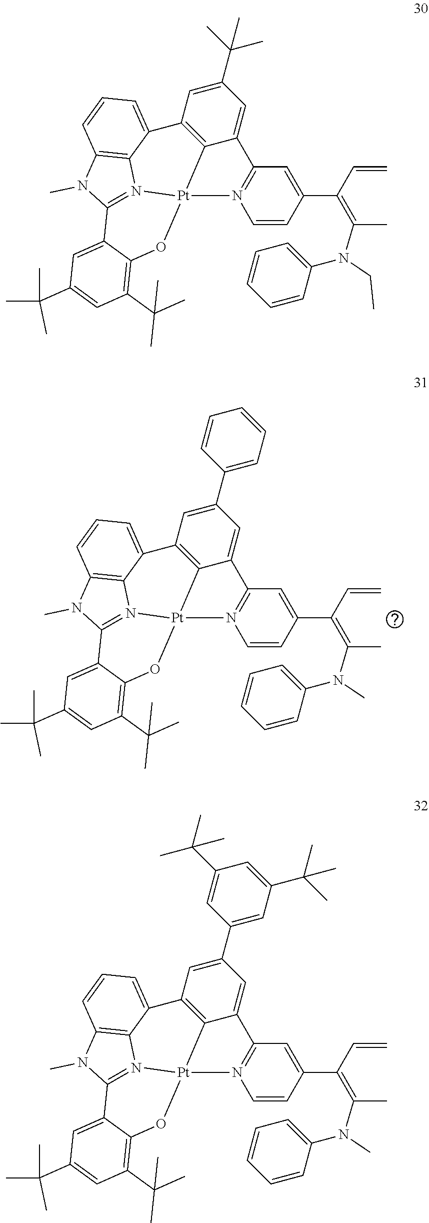

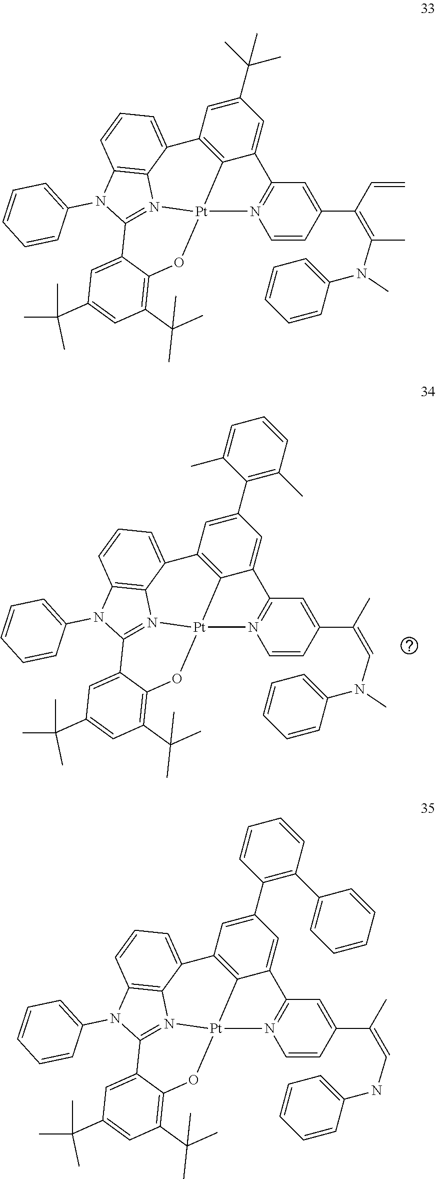

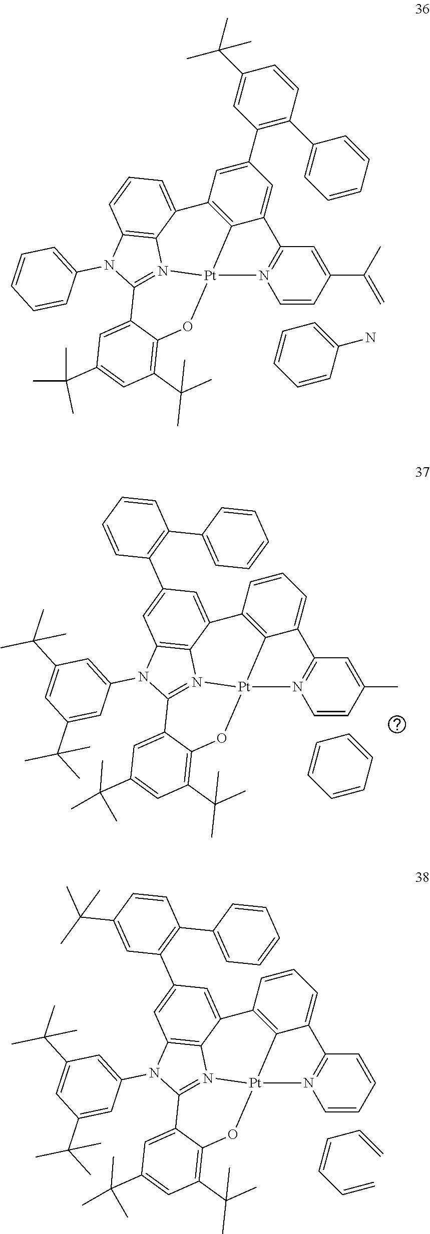

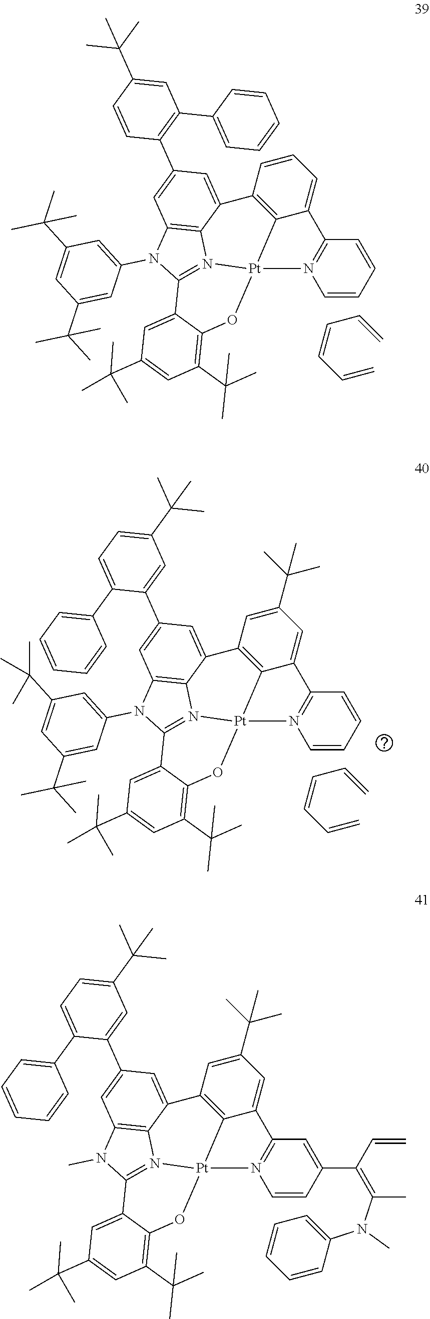

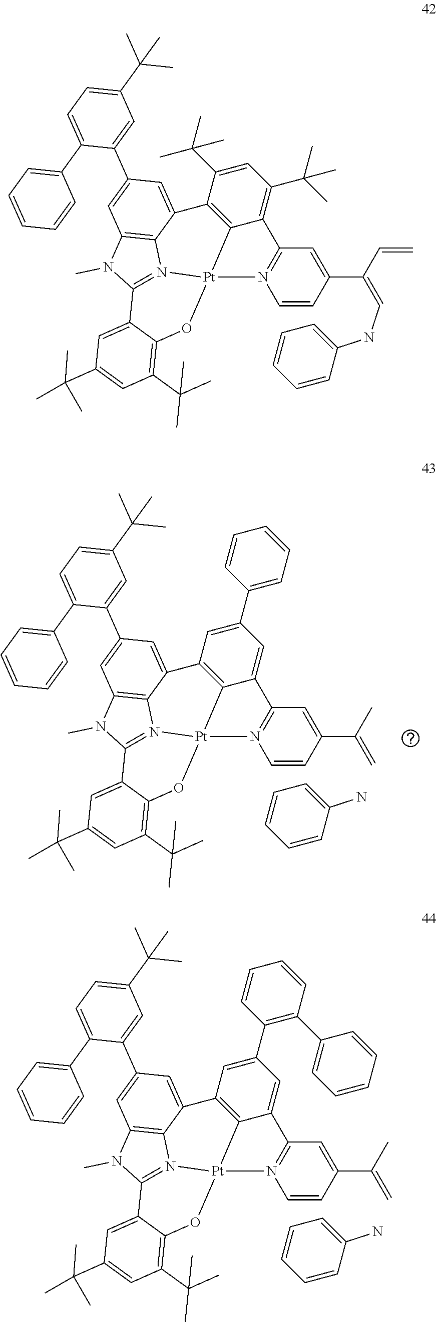

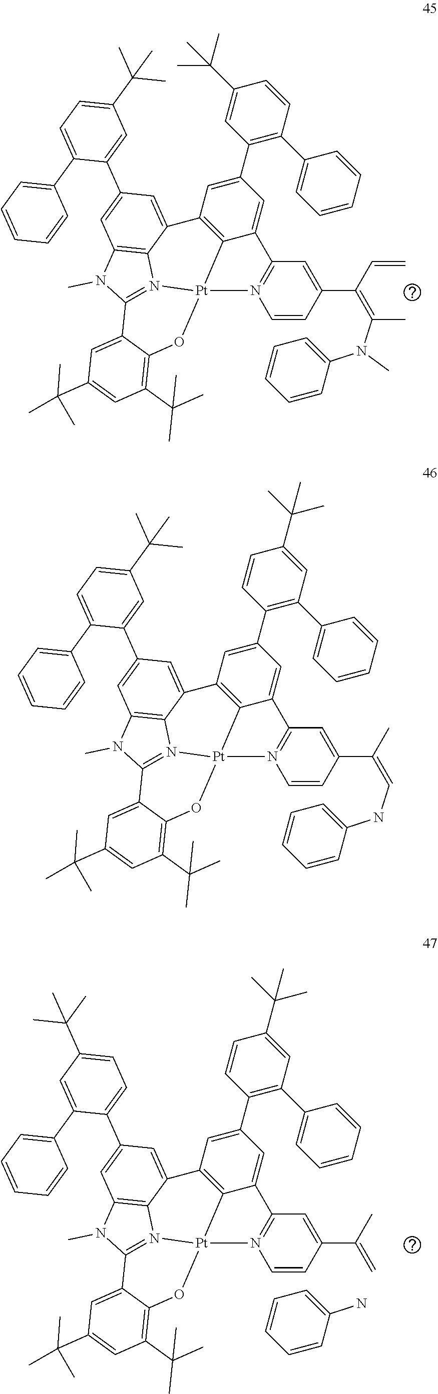

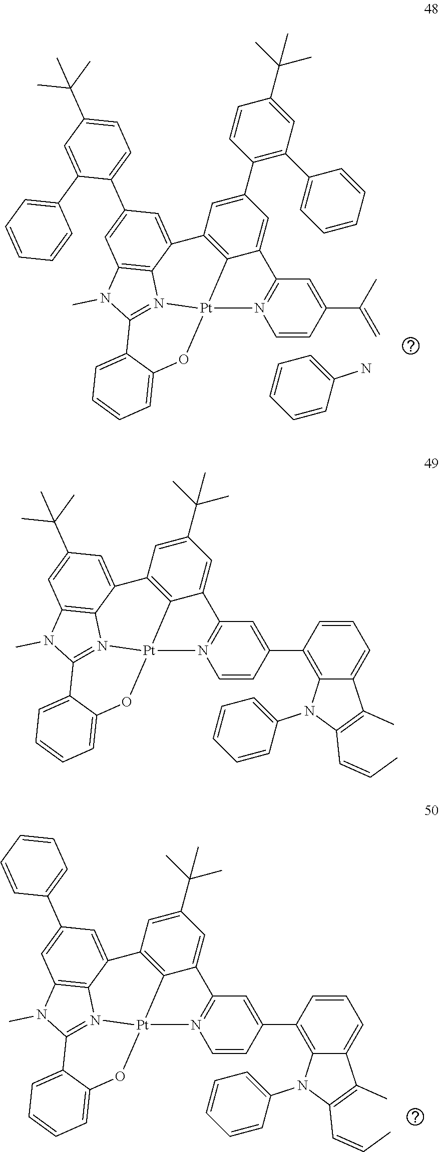

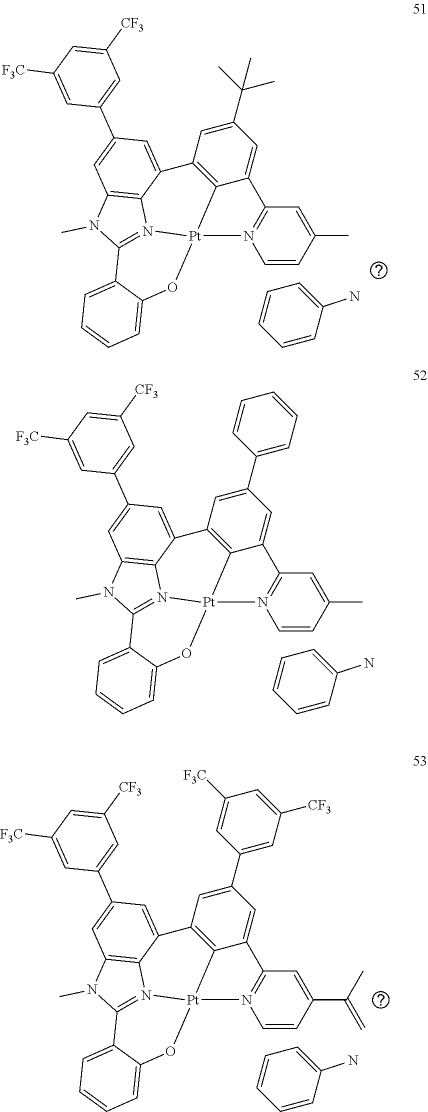

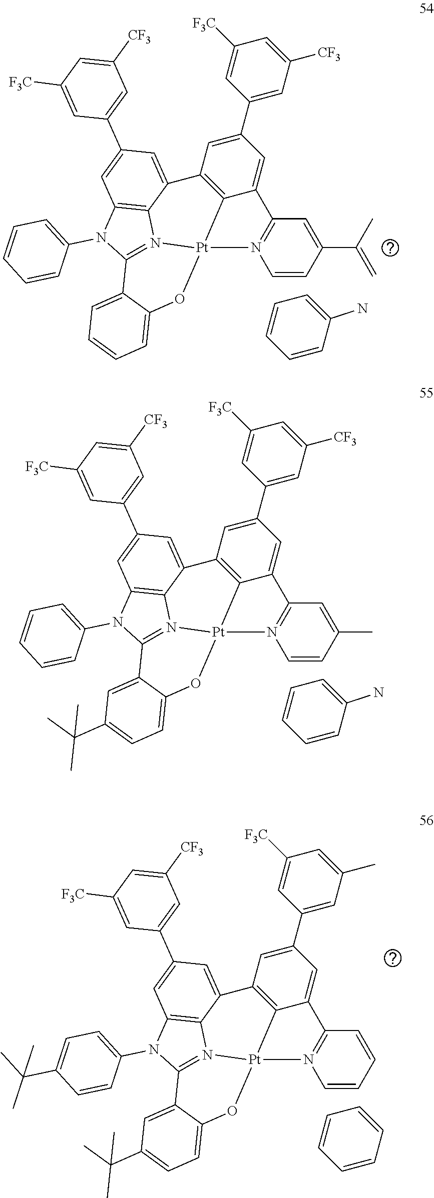

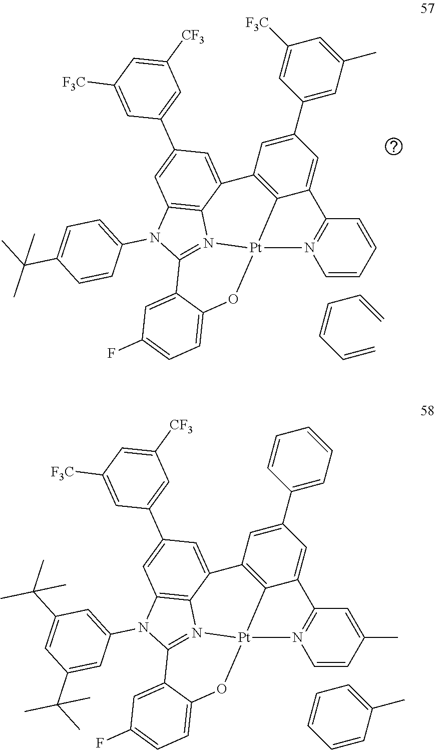

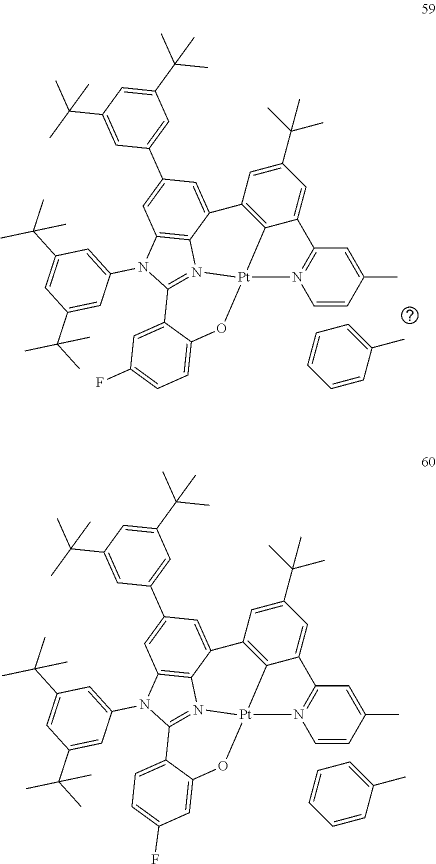

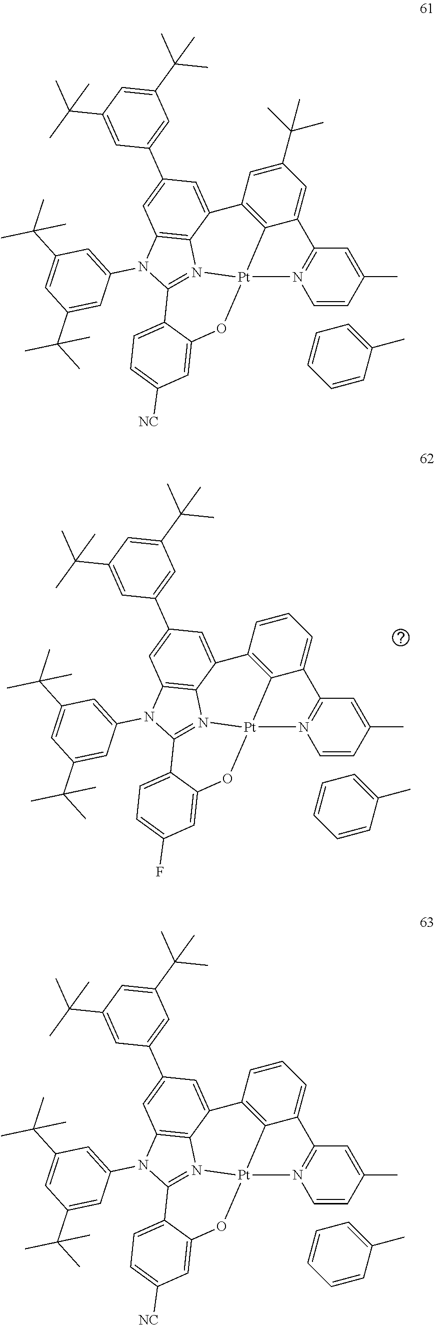

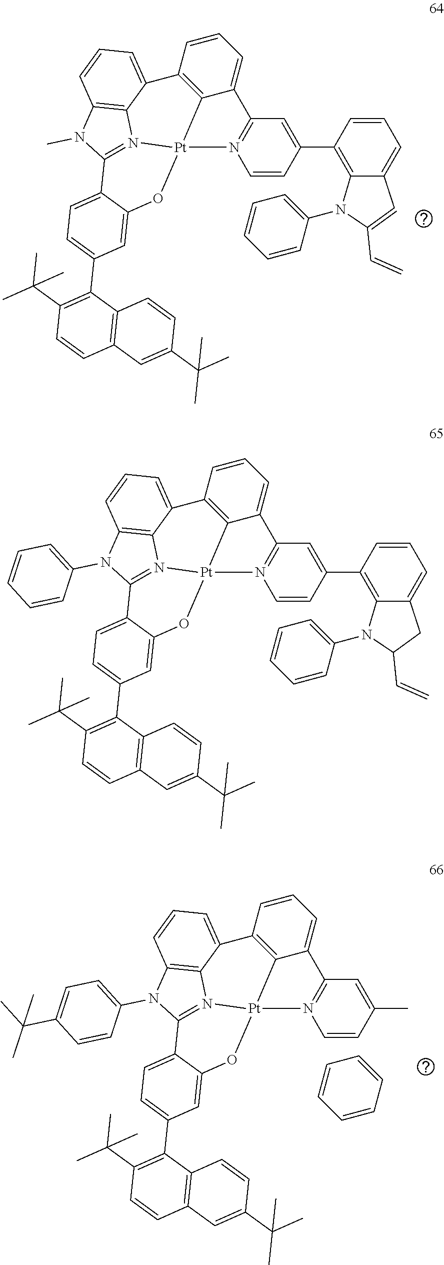

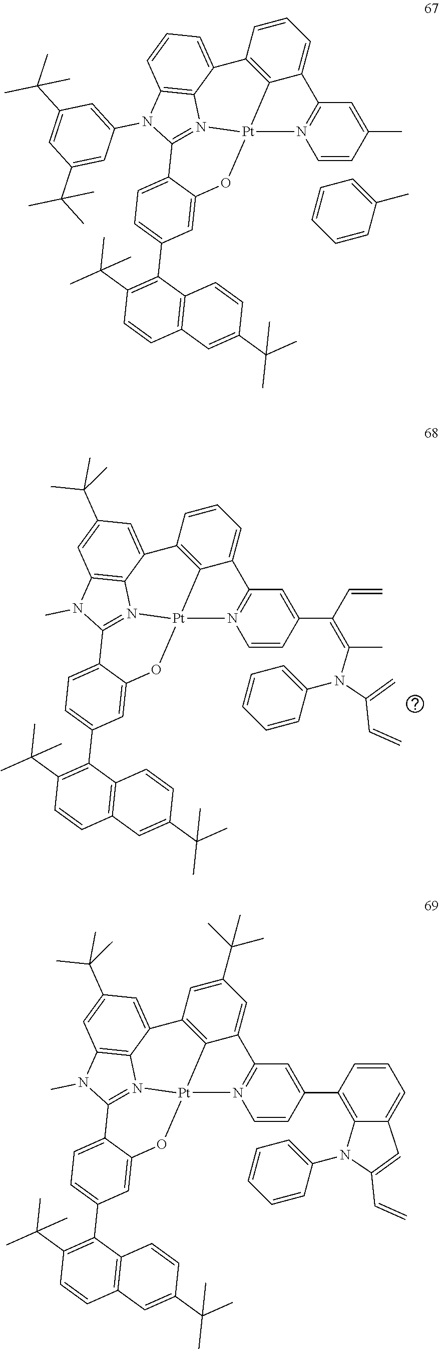

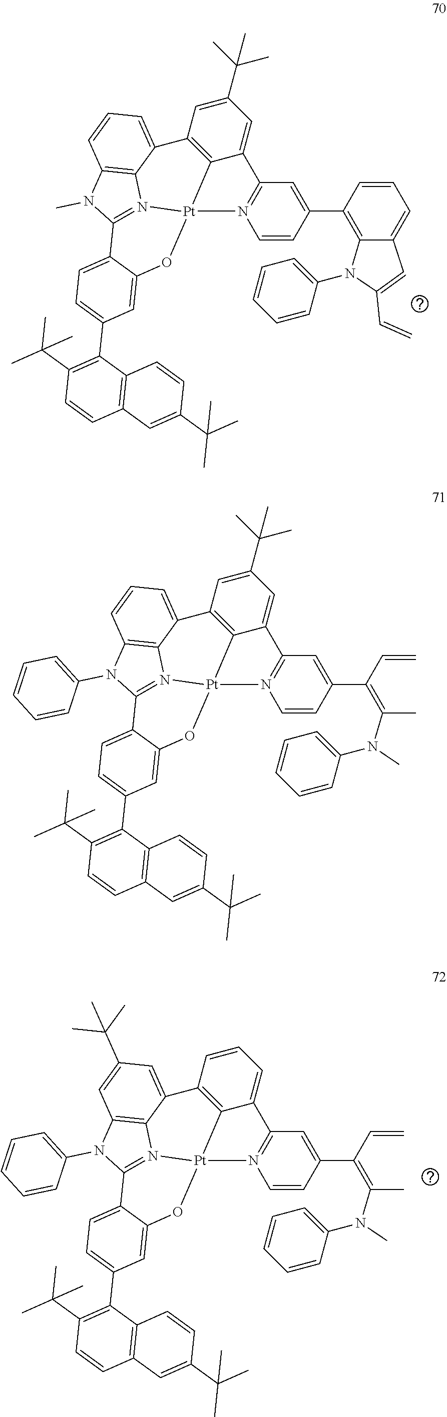

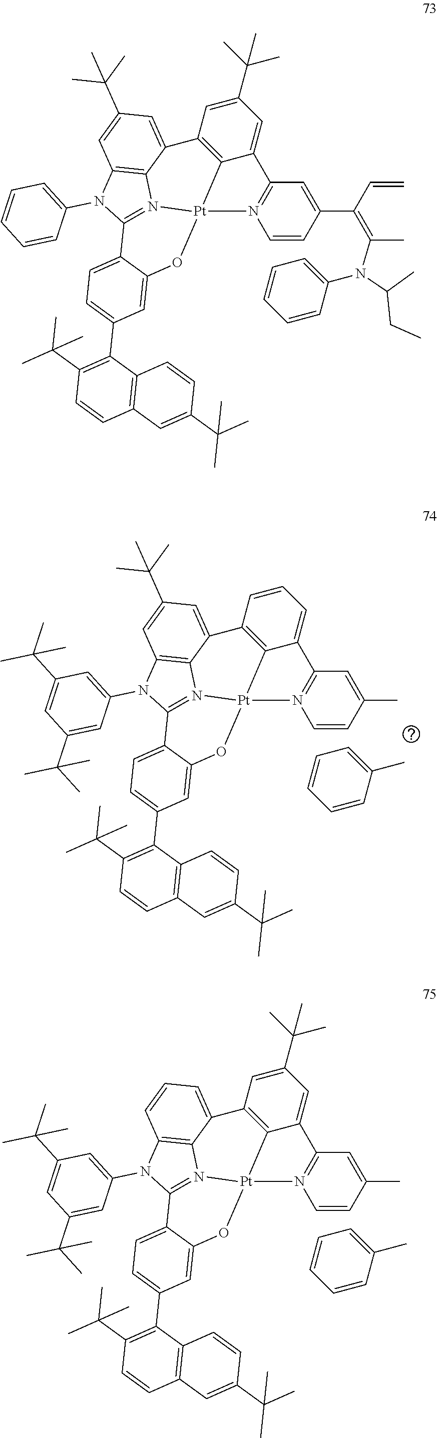

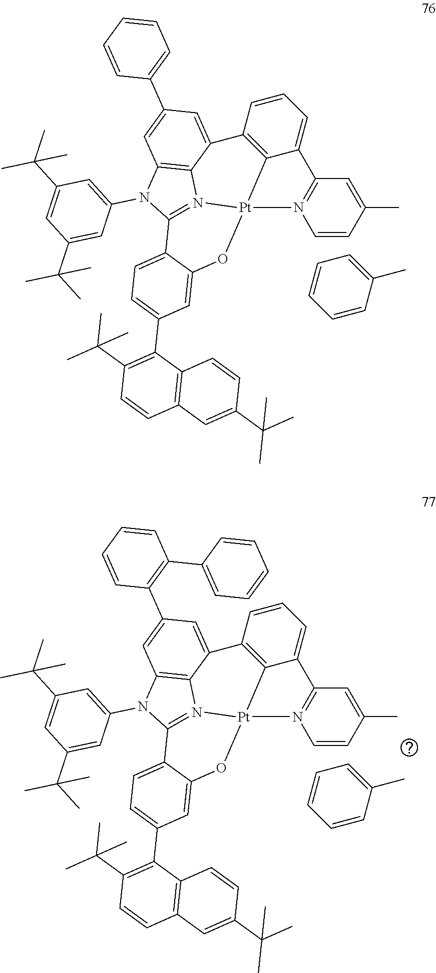

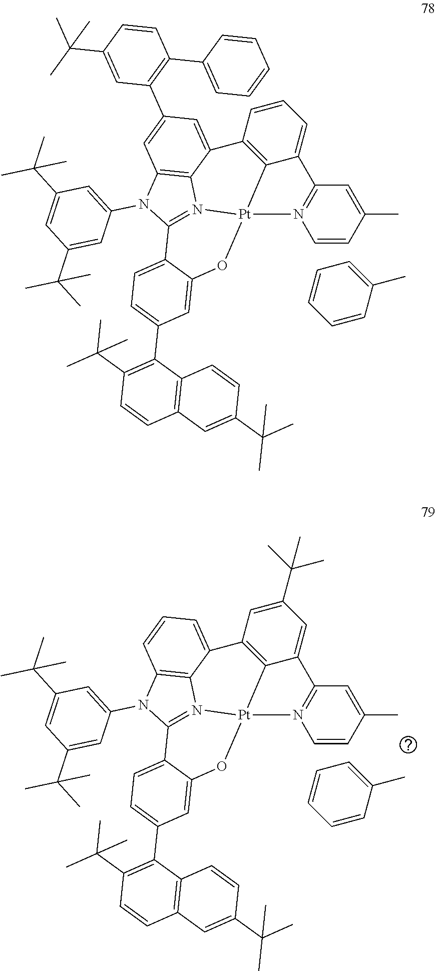

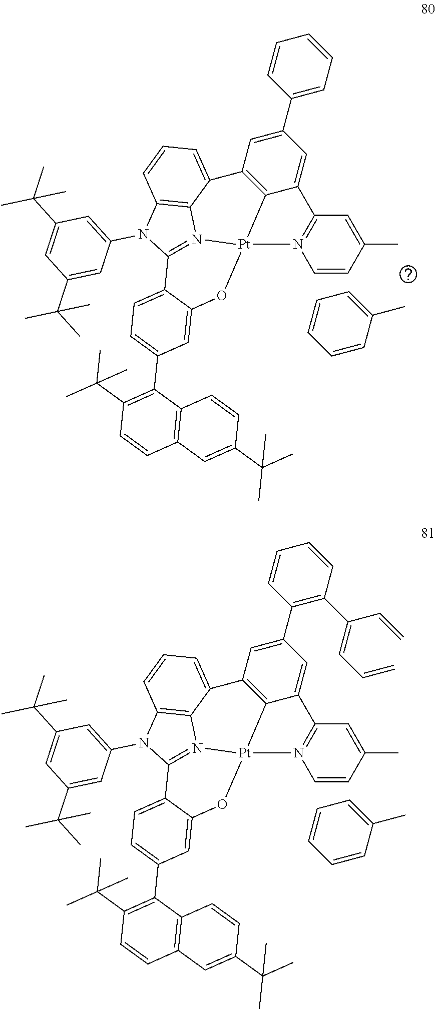

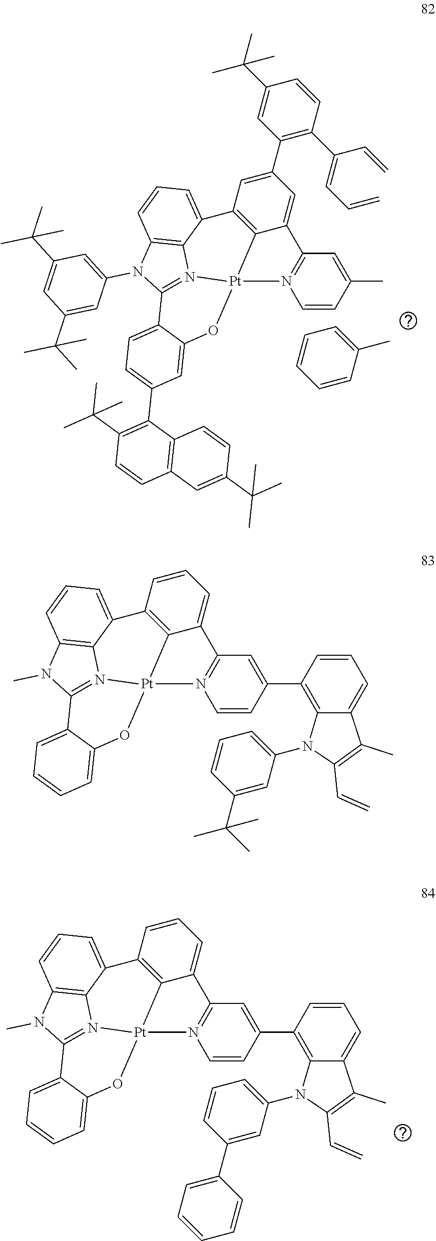

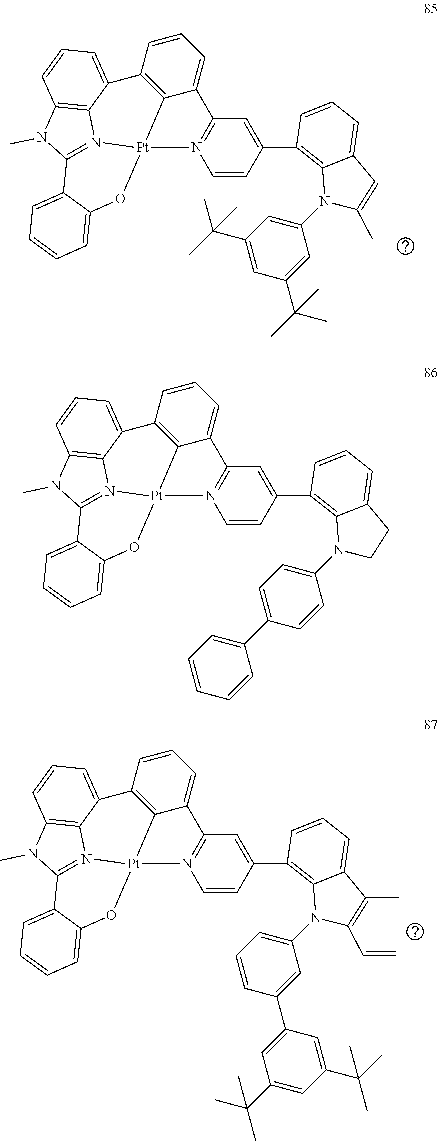

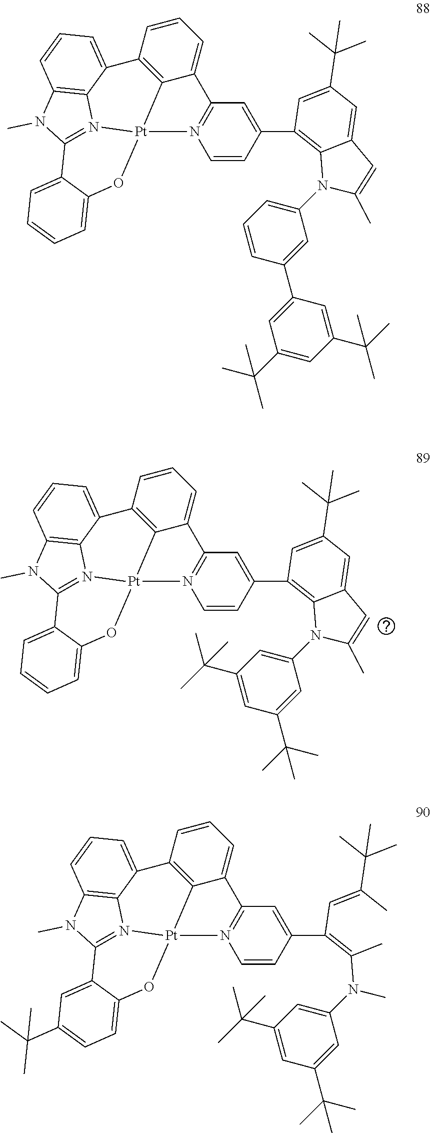

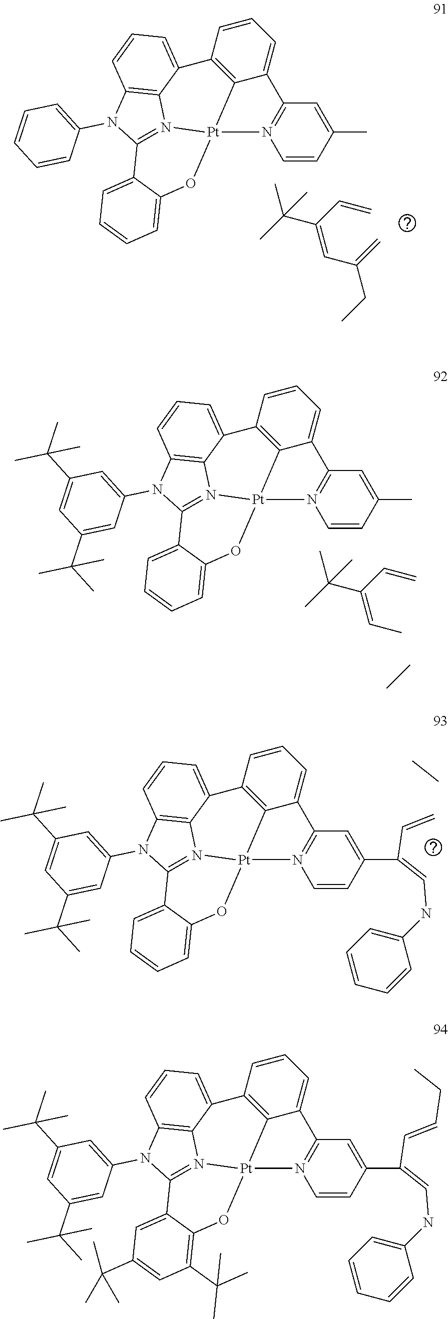

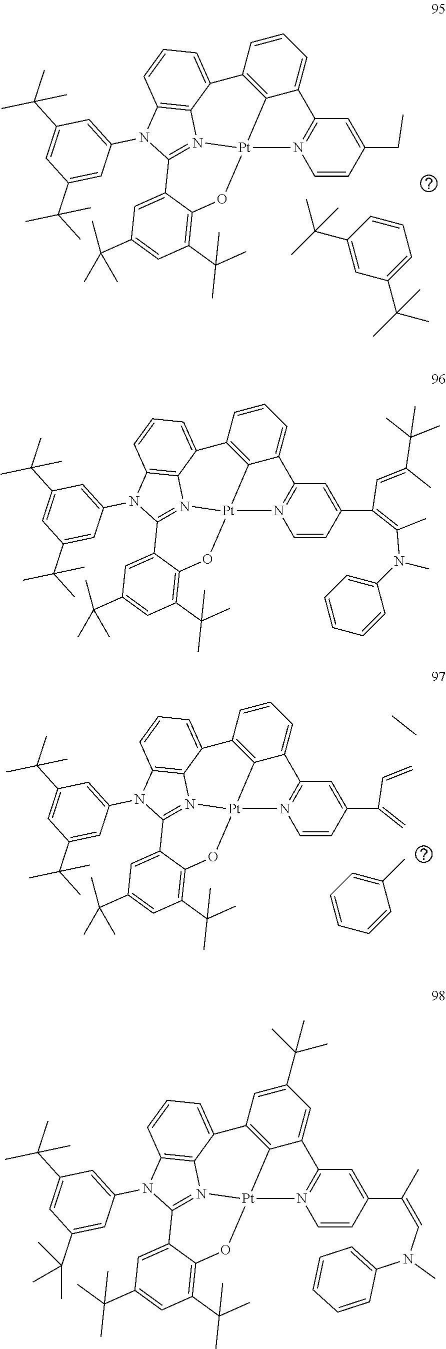

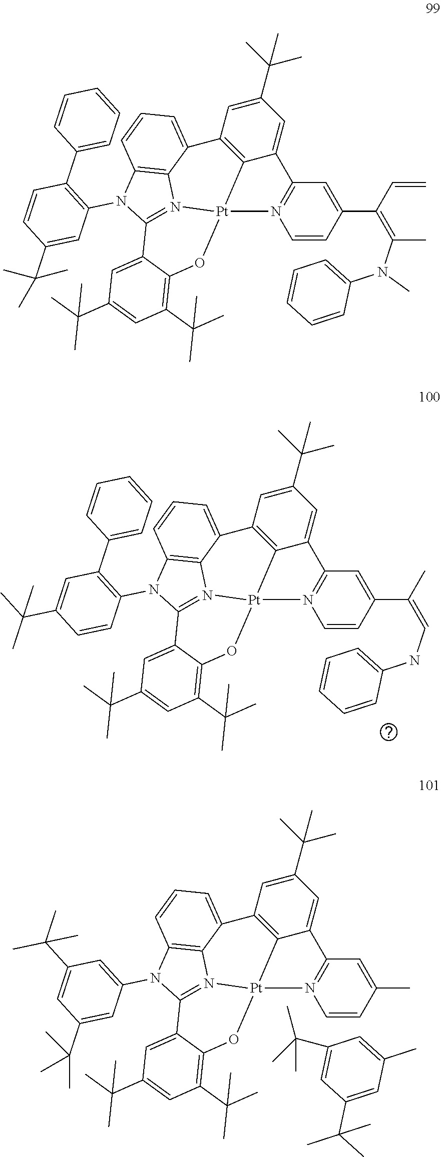

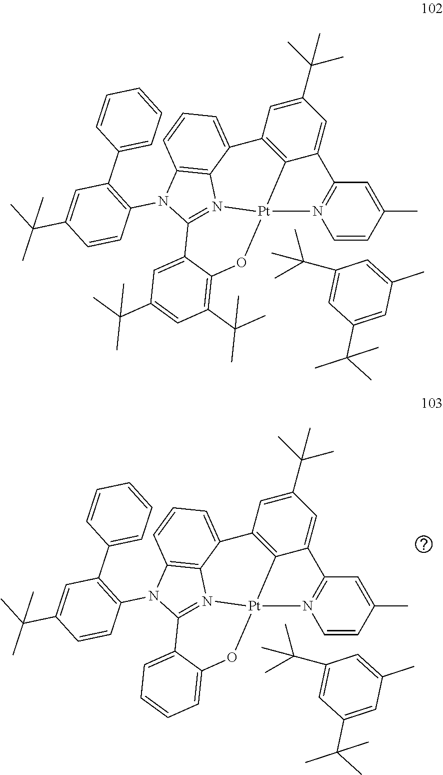

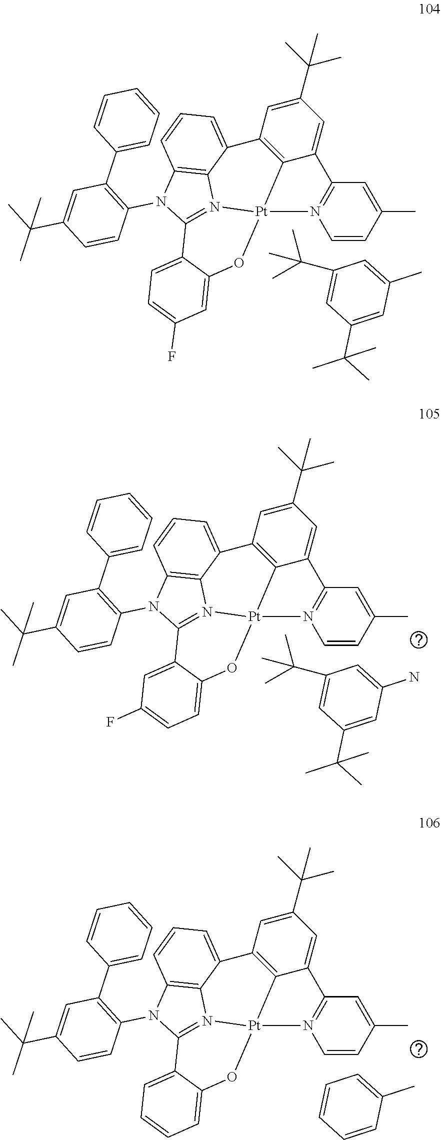

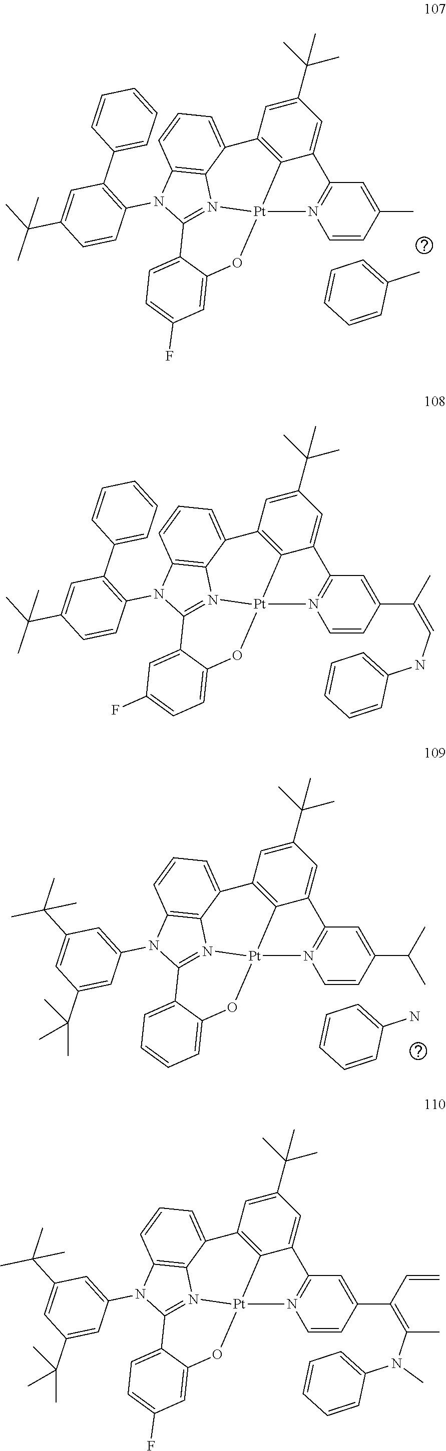

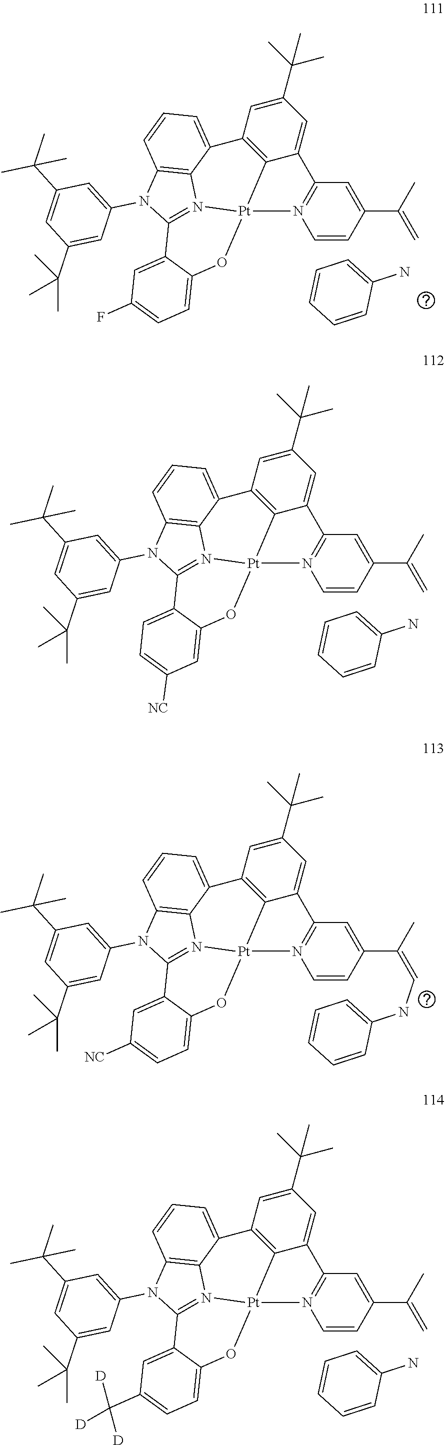

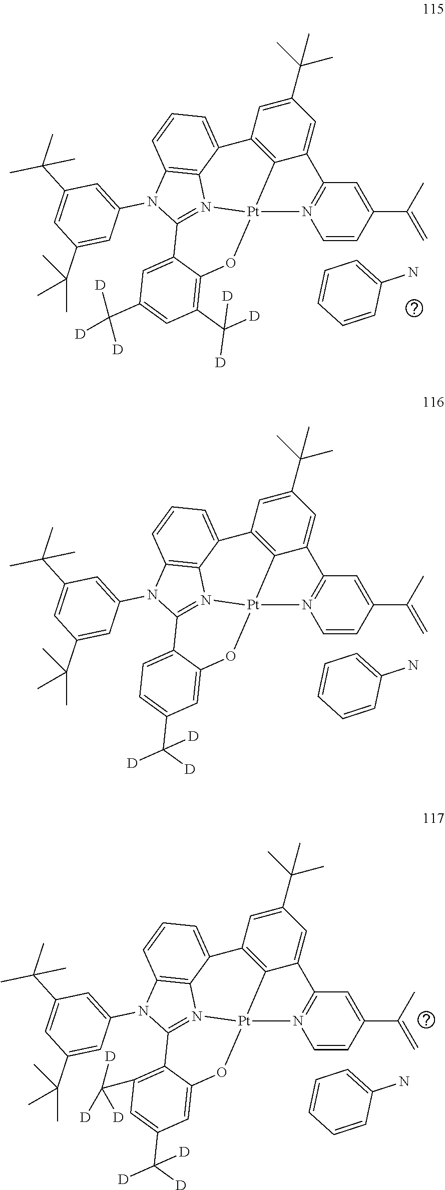

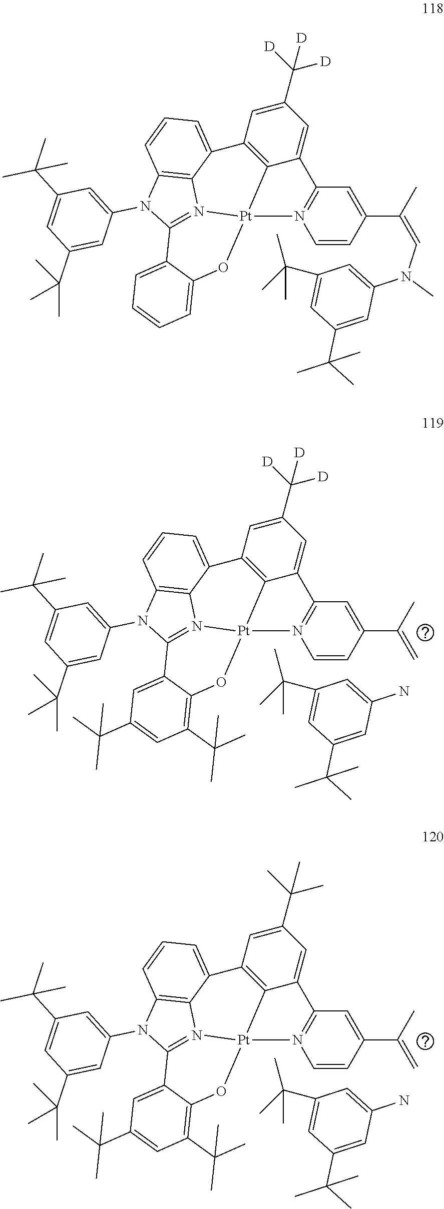

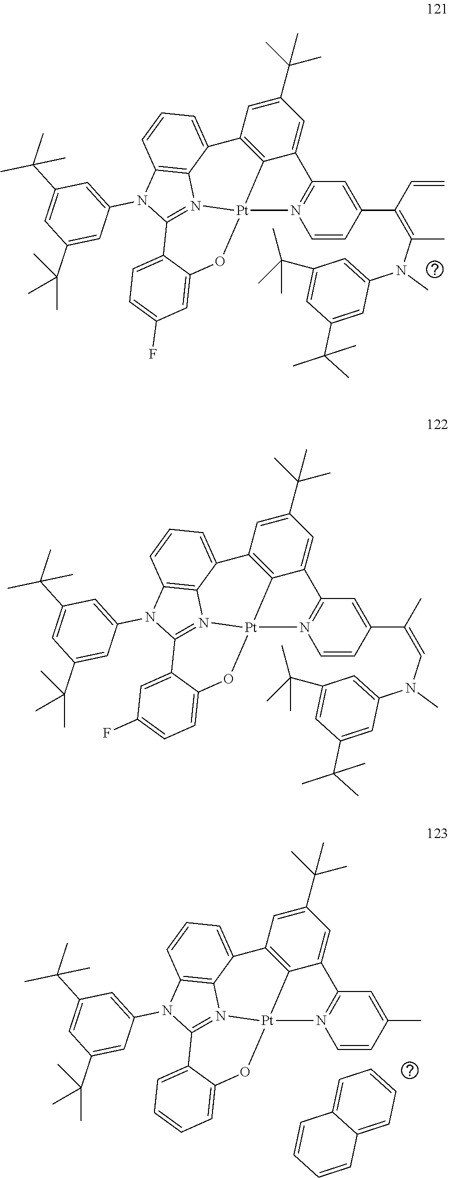

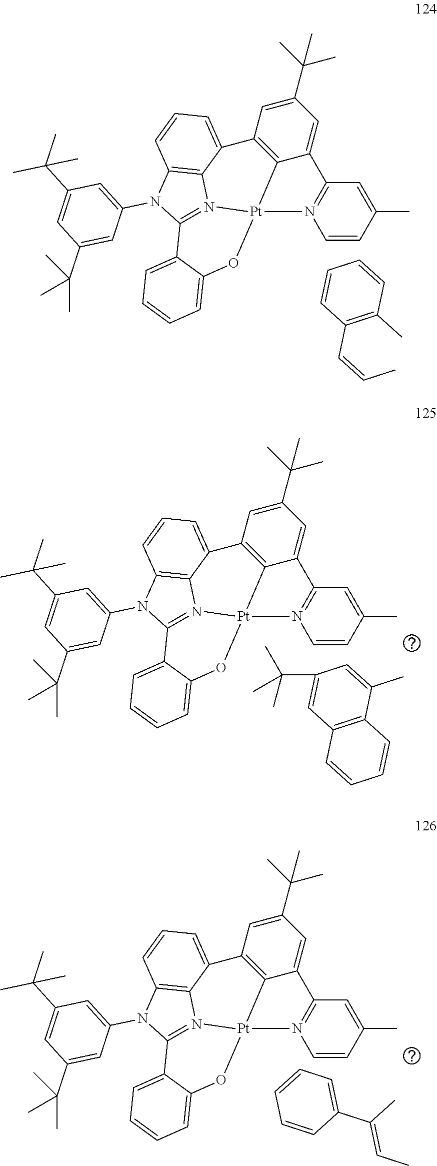

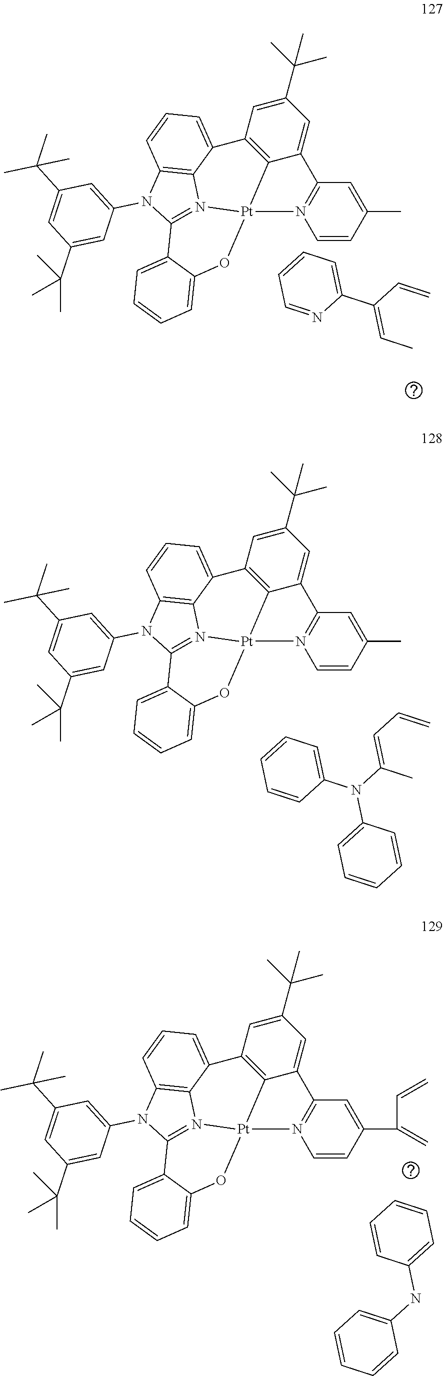

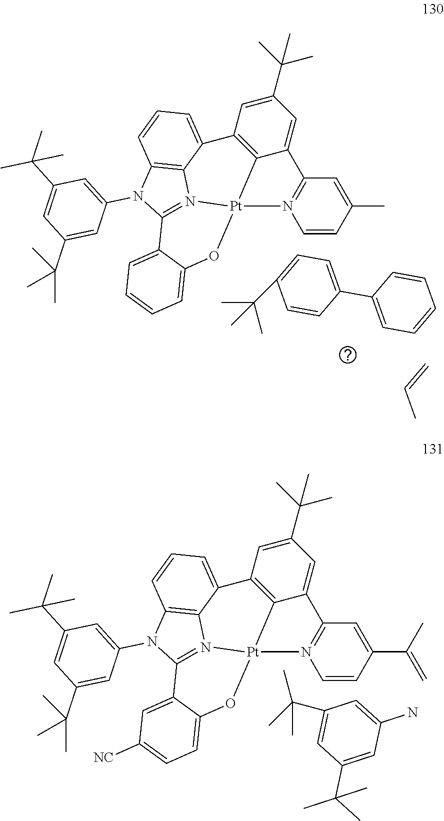

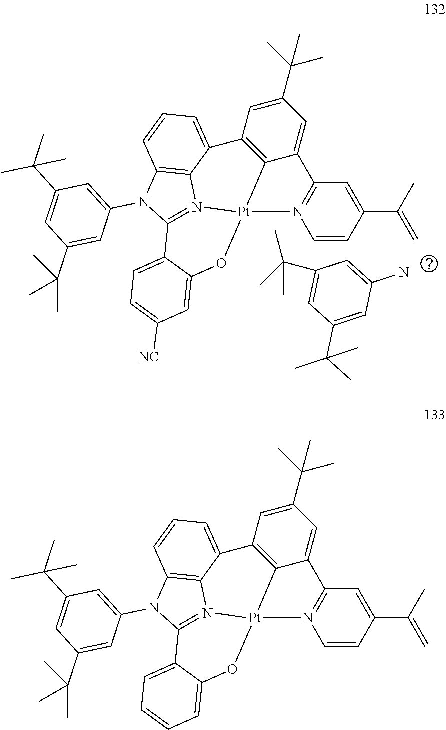

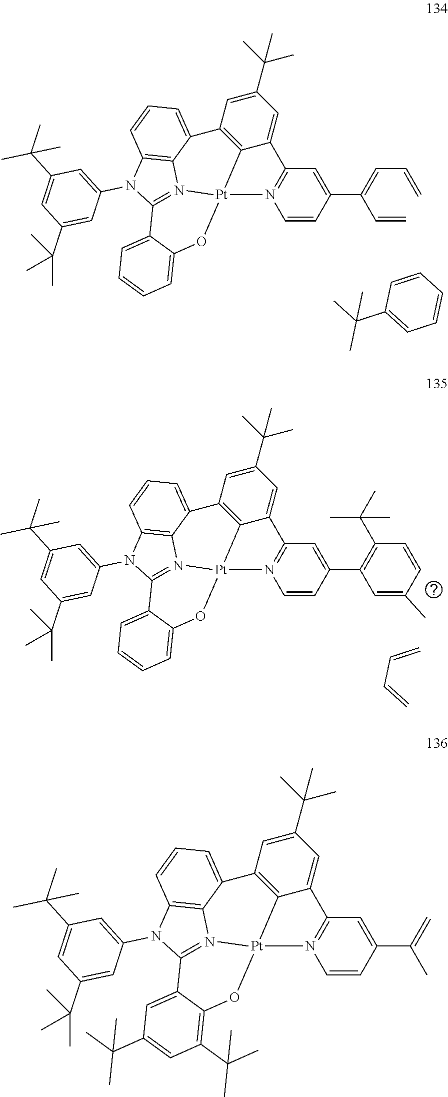

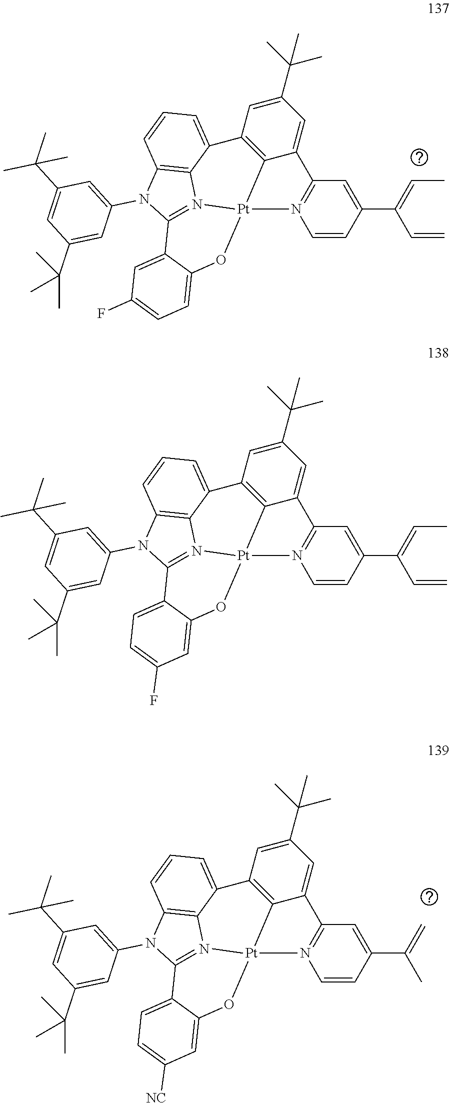

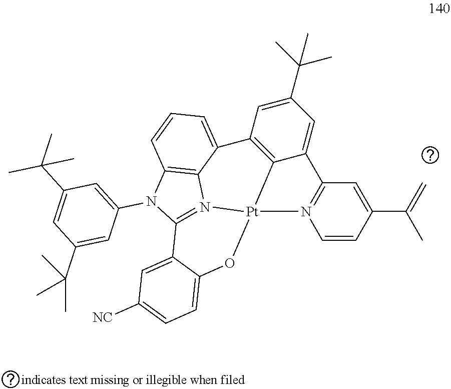

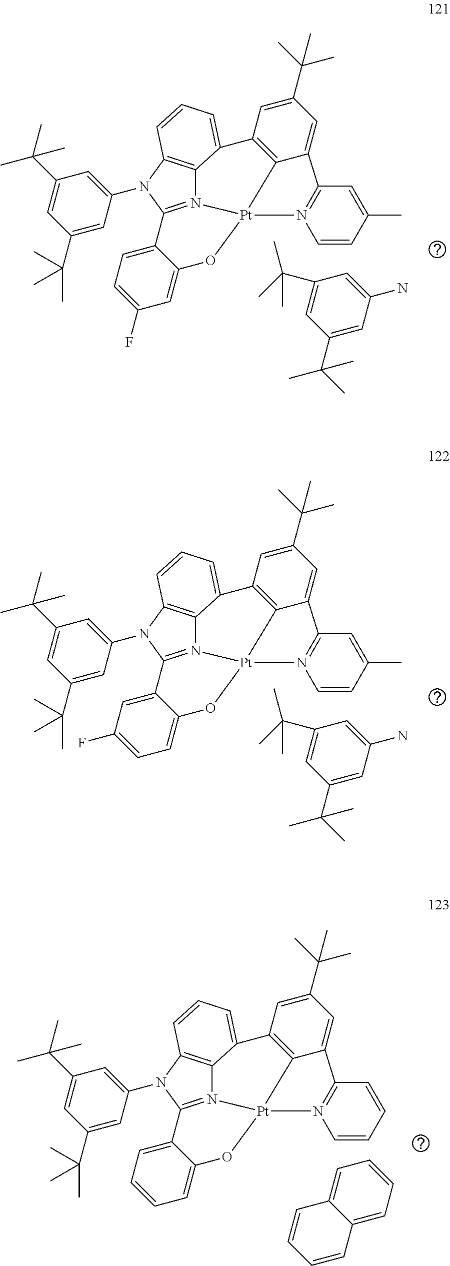

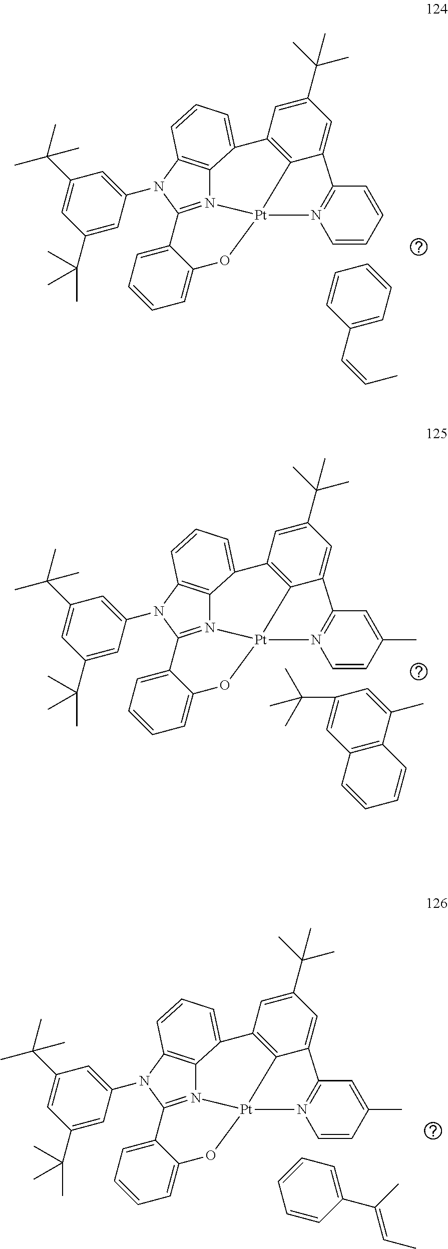

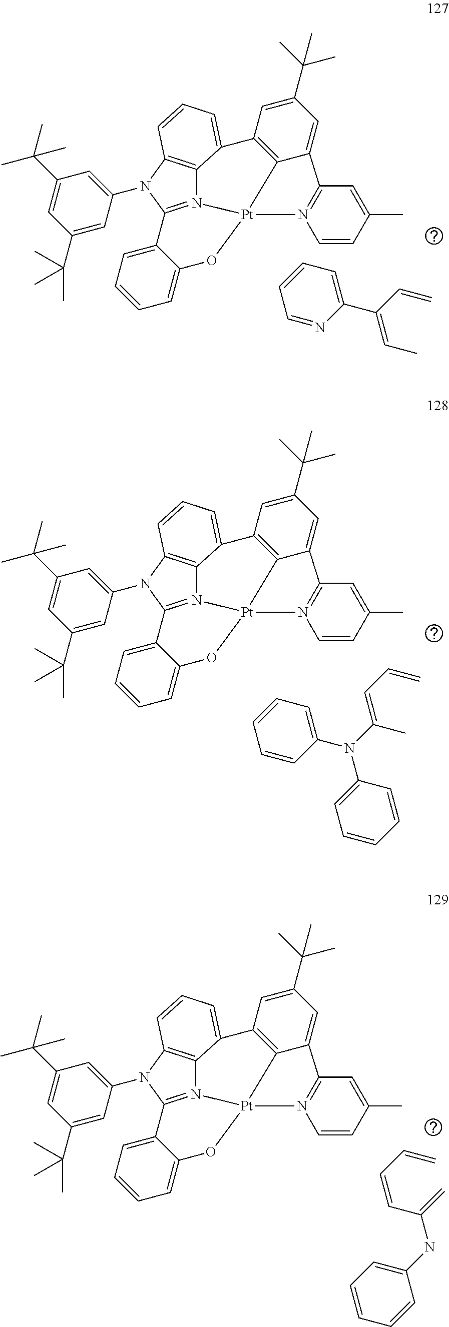

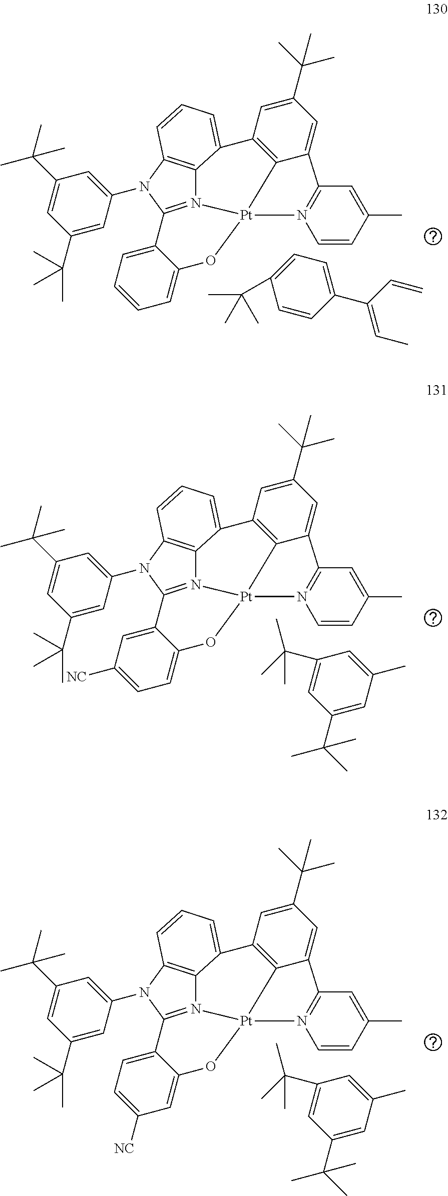

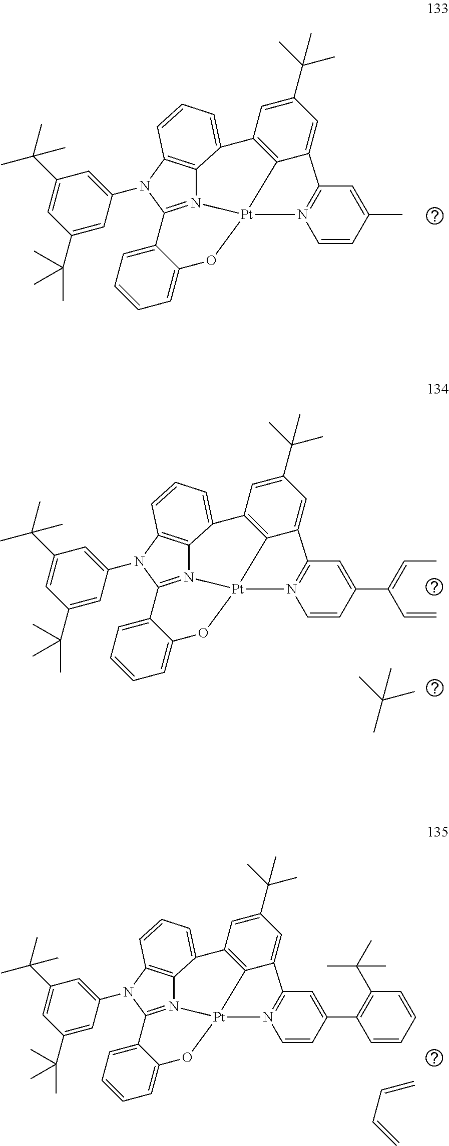

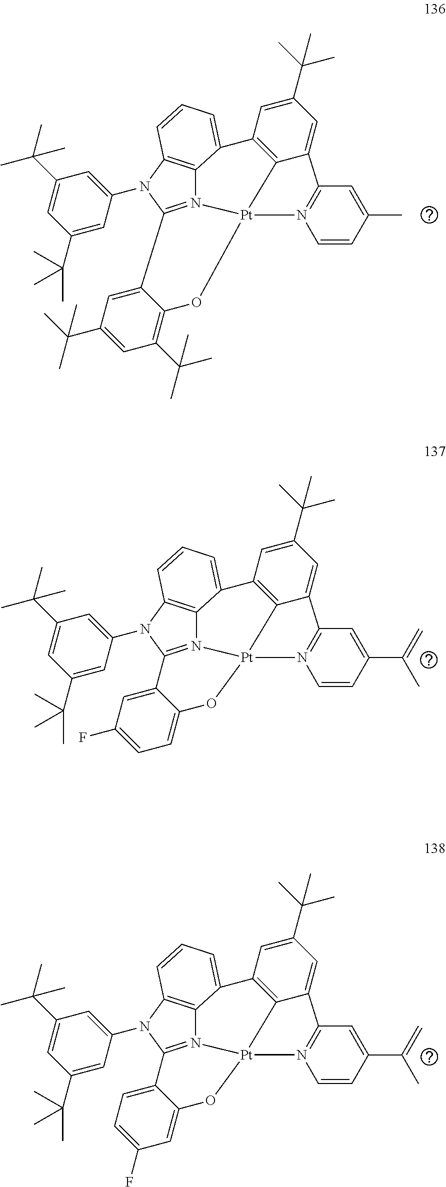

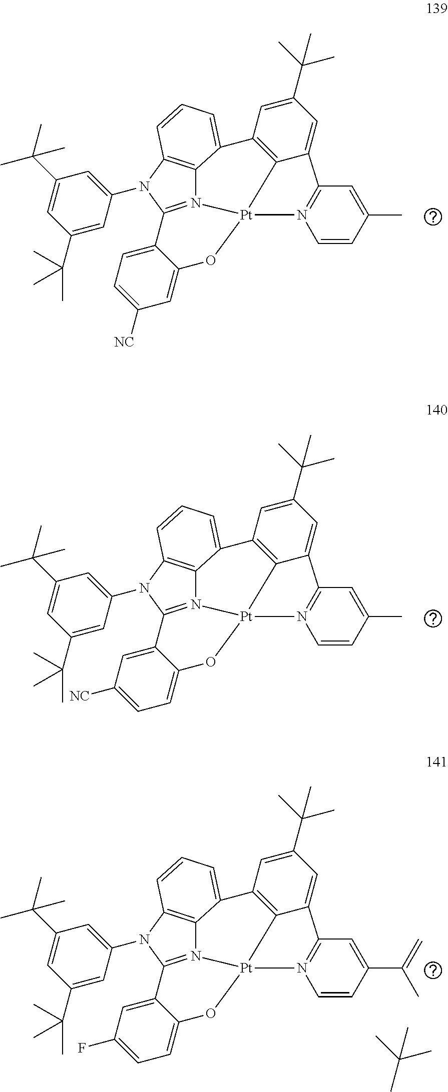

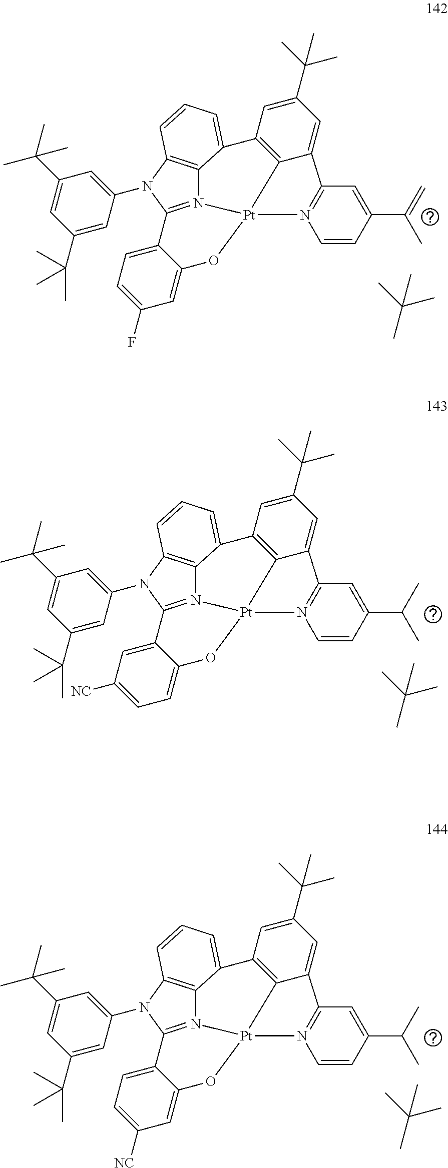

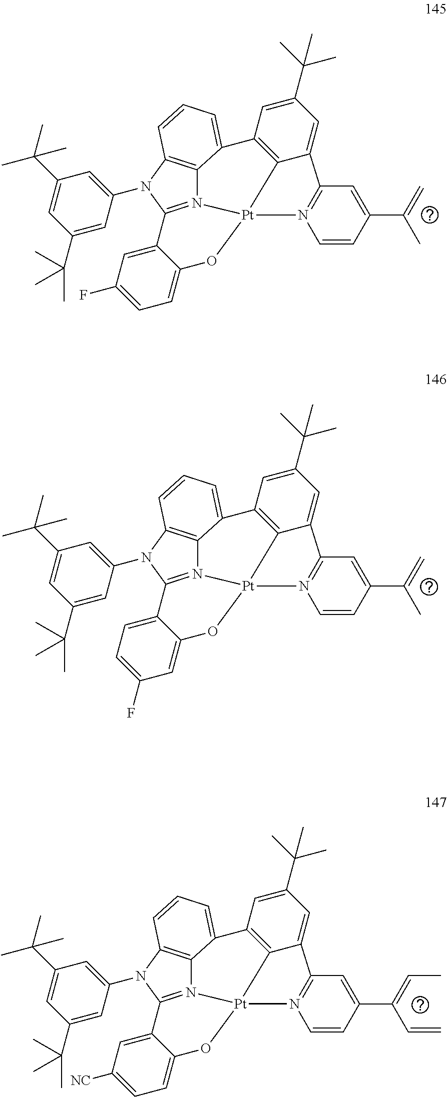

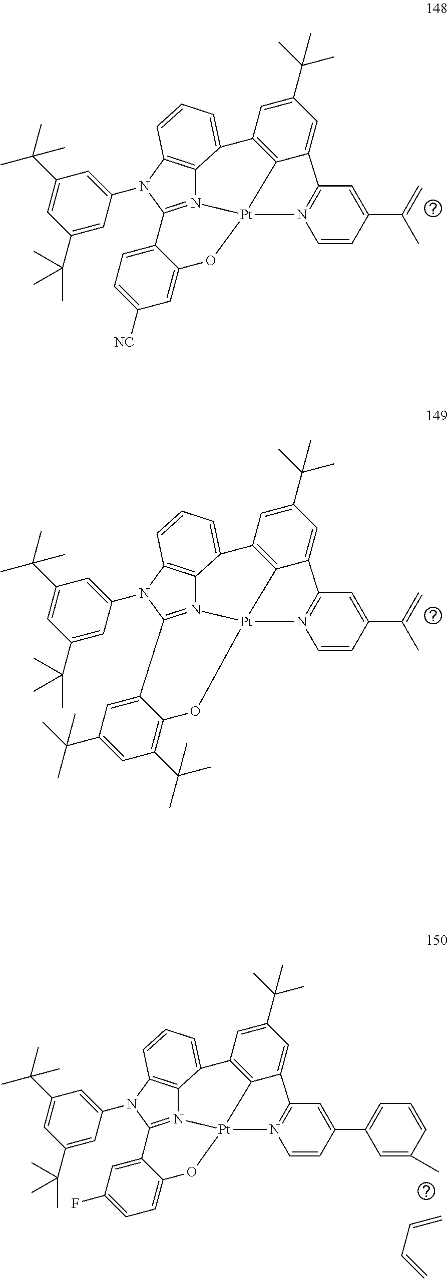

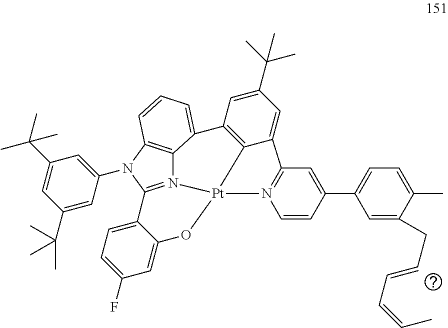

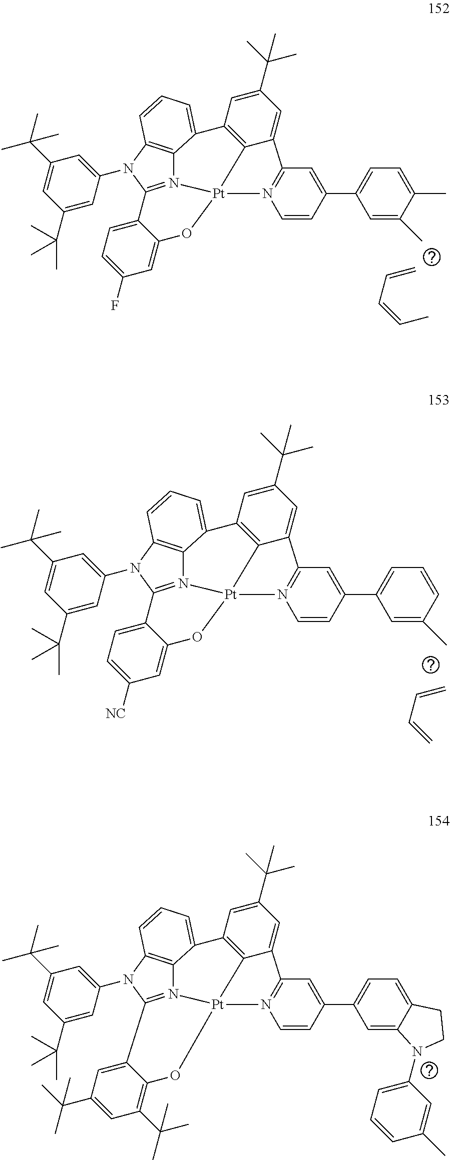

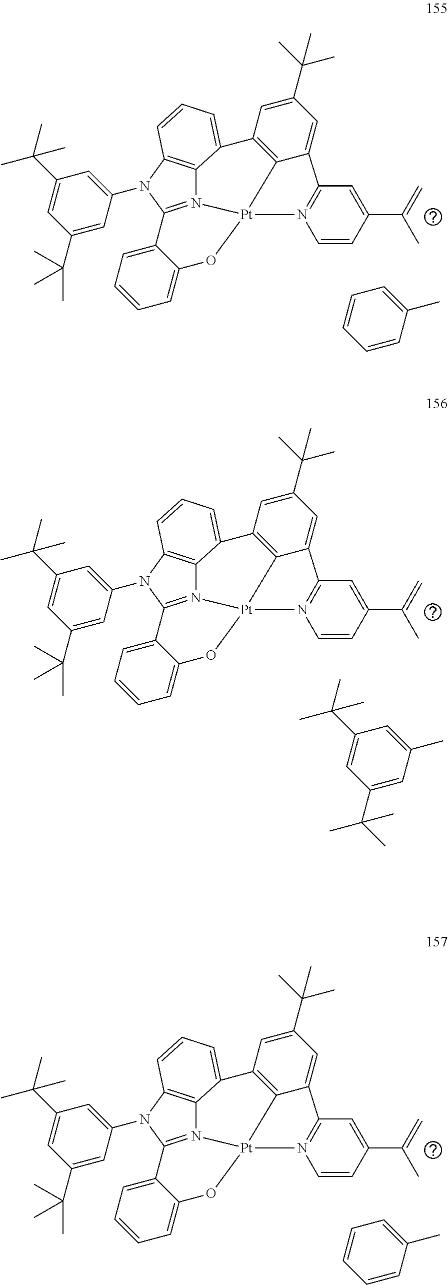

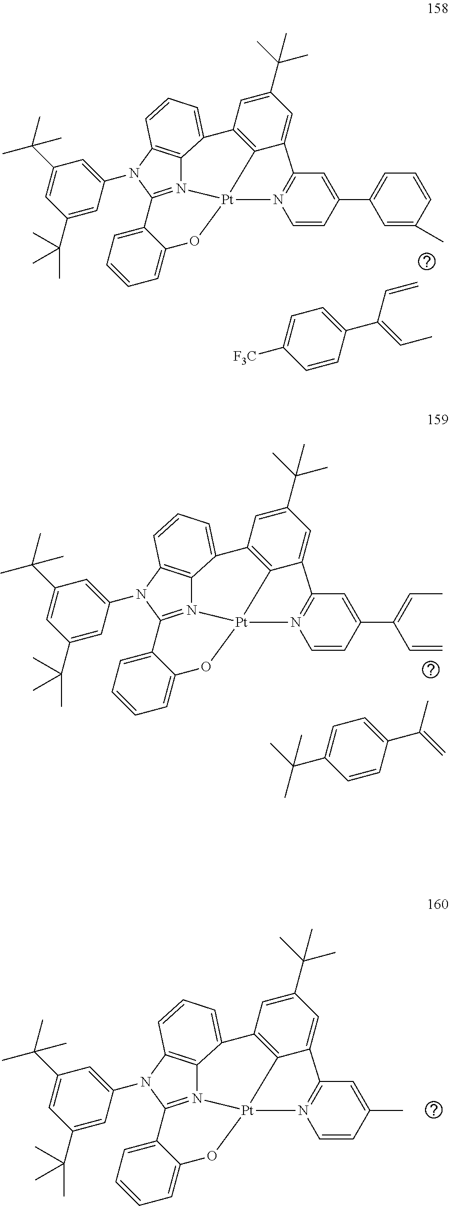

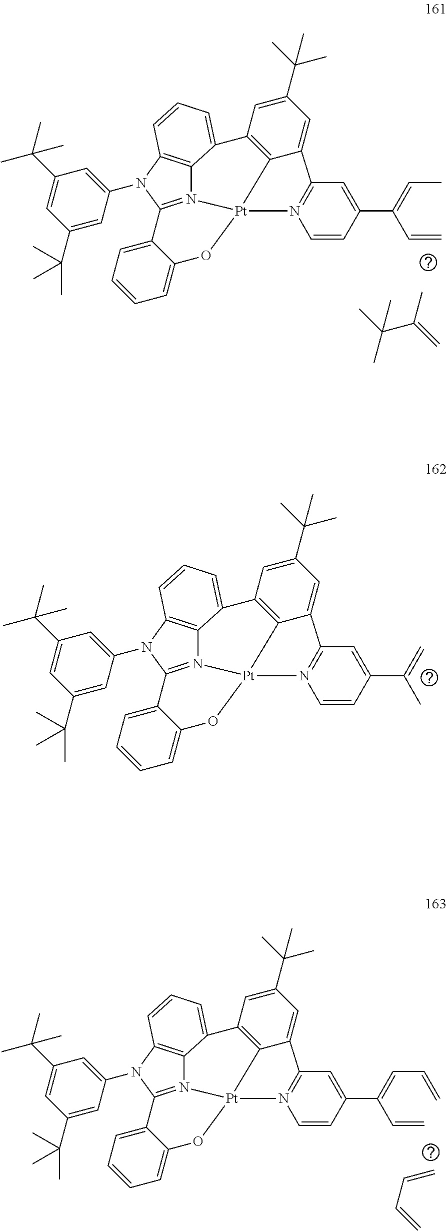

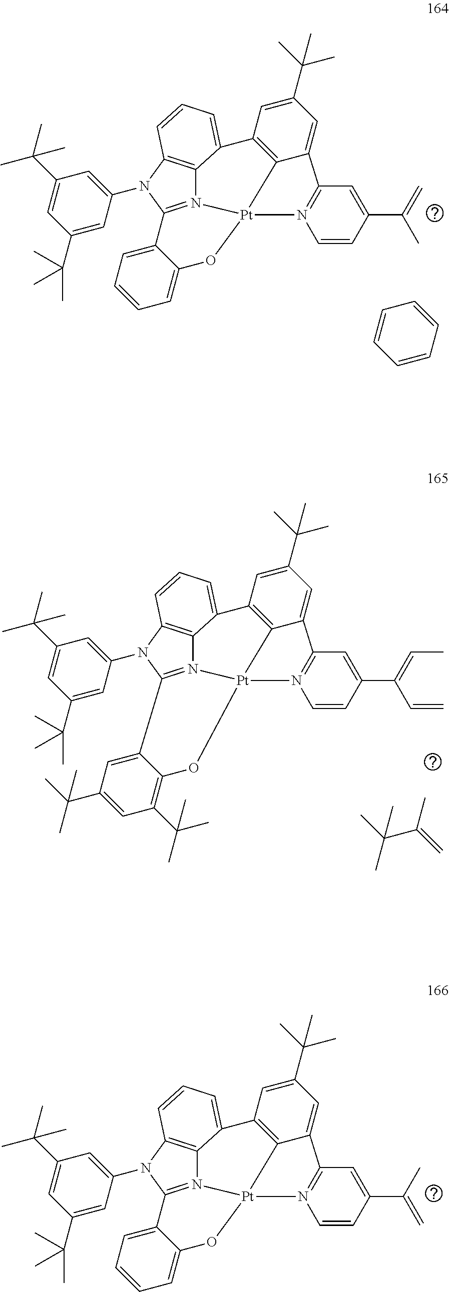

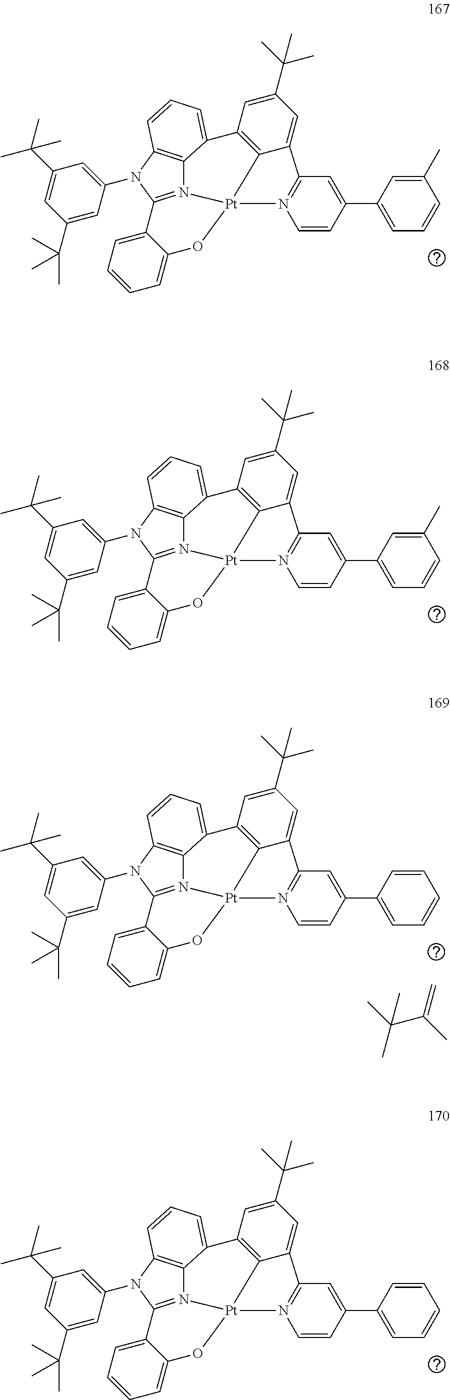

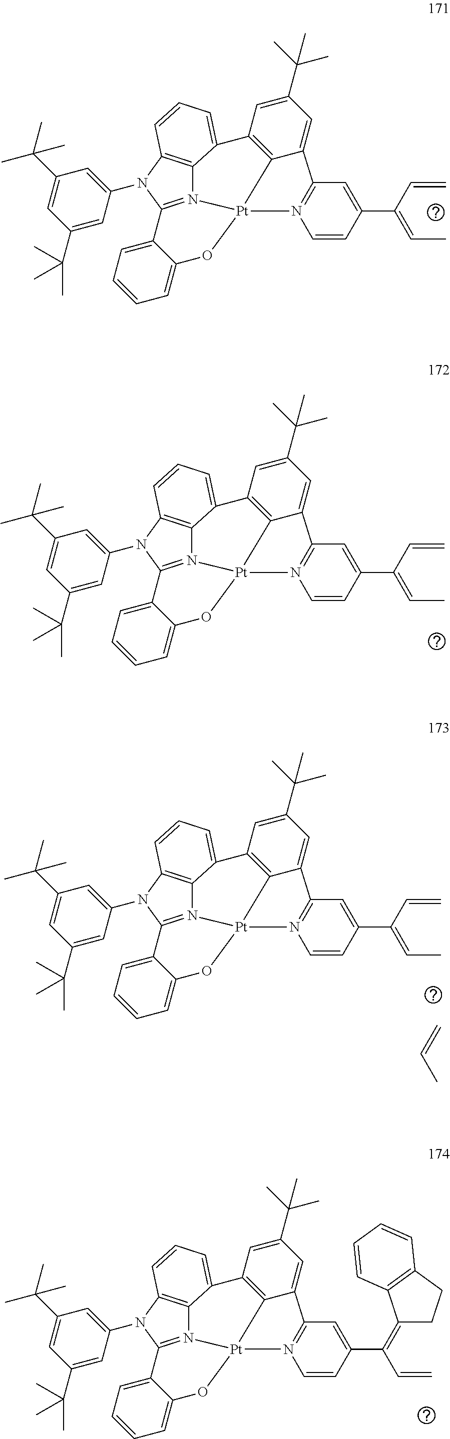

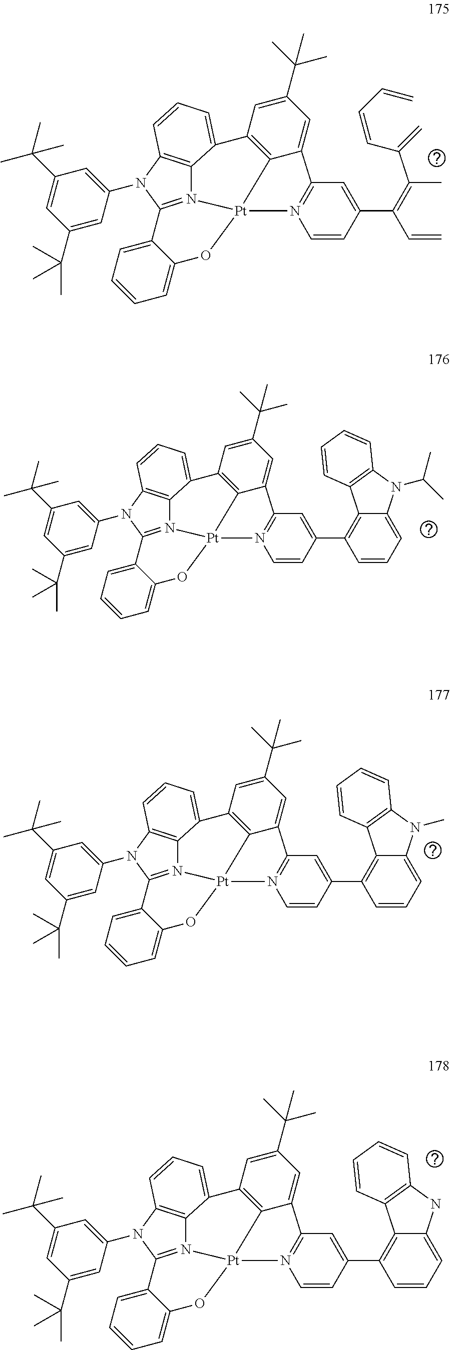

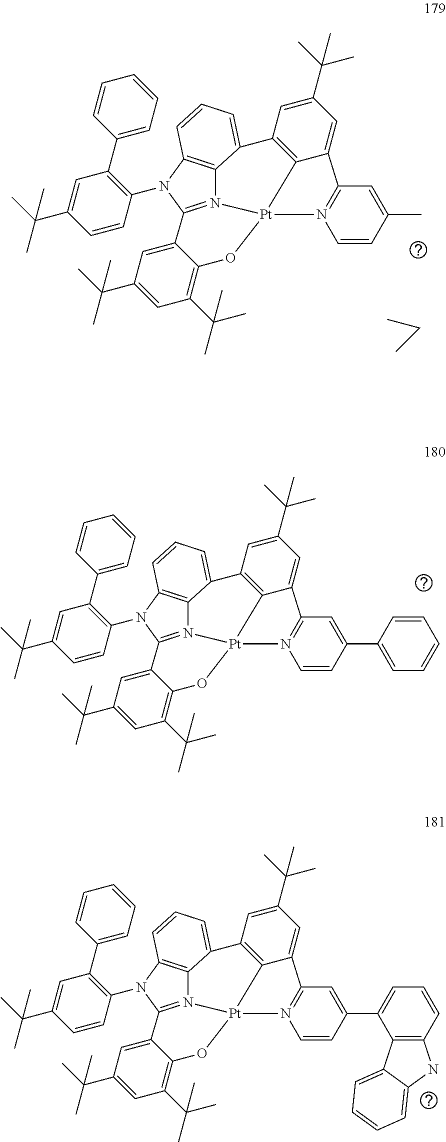

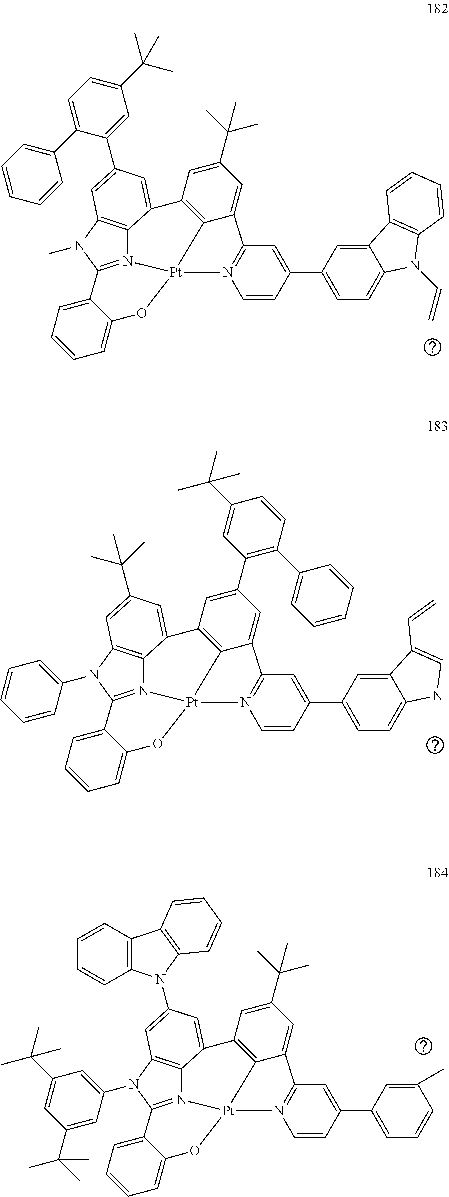

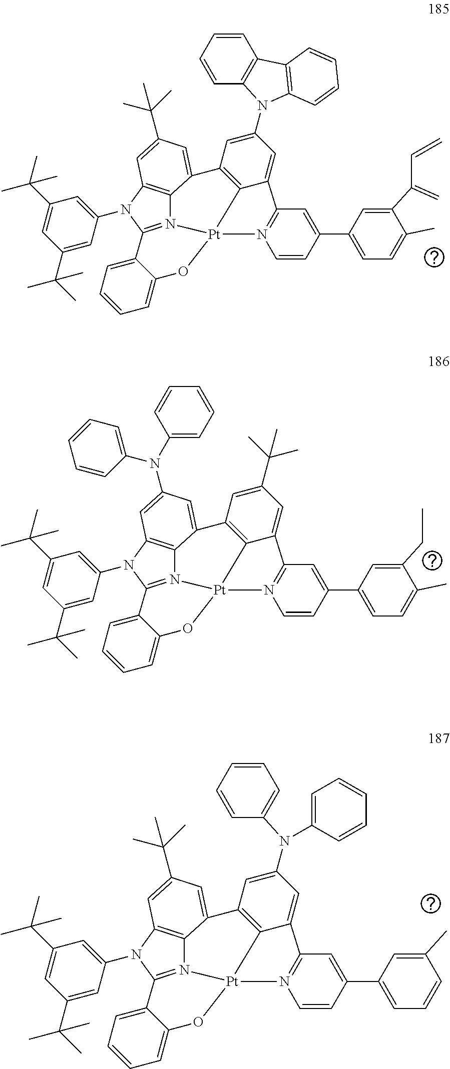

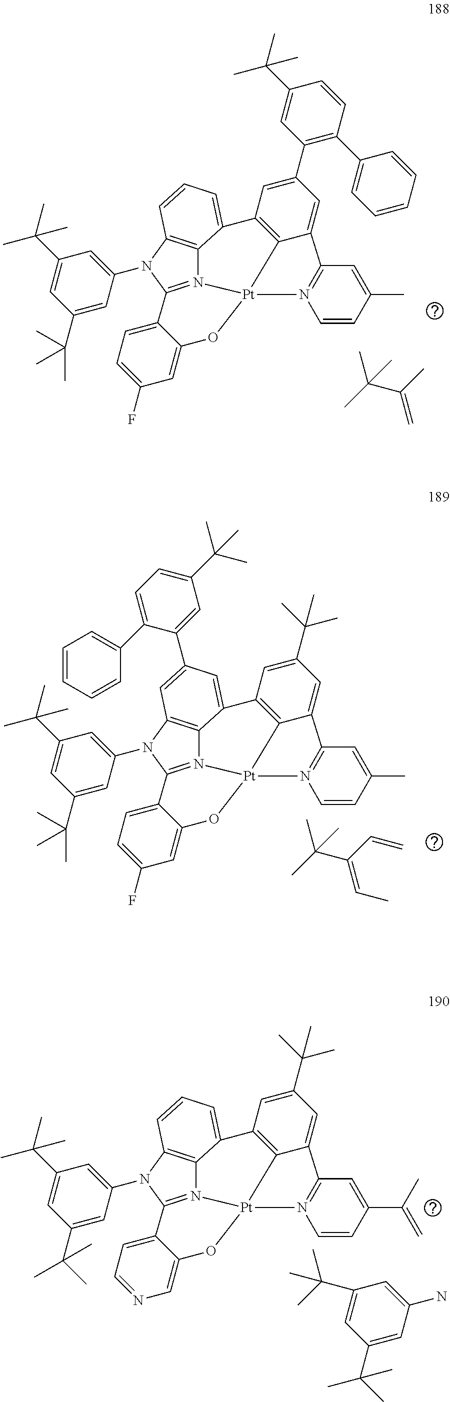

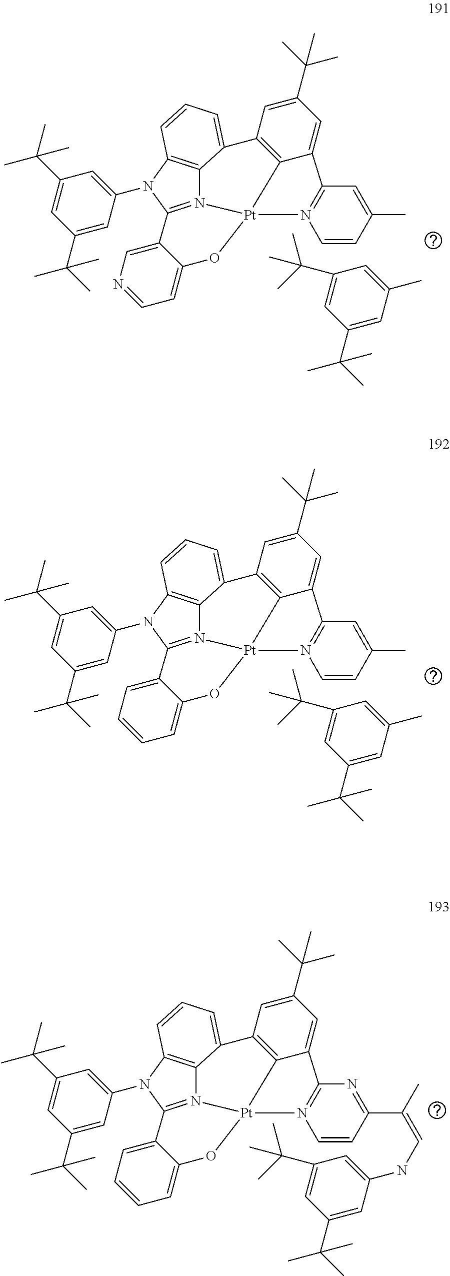

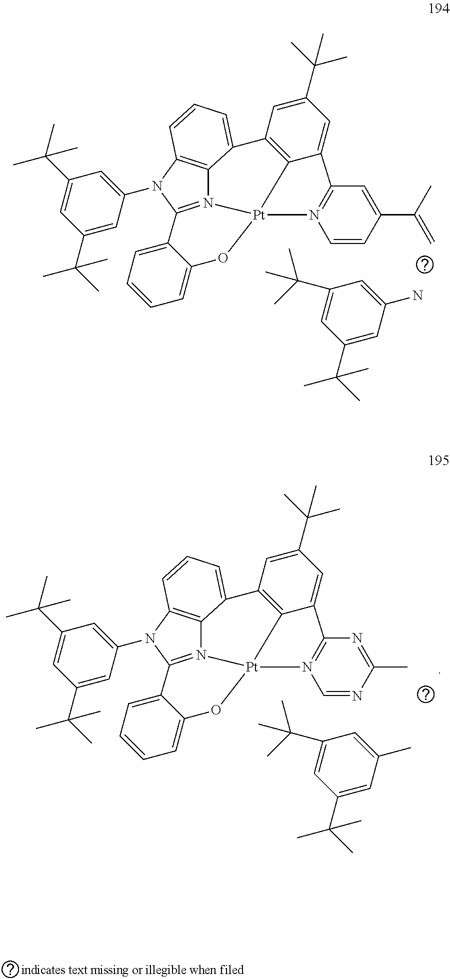

8. A ligand of the platinum complex of claim 1, having the following structural formula:

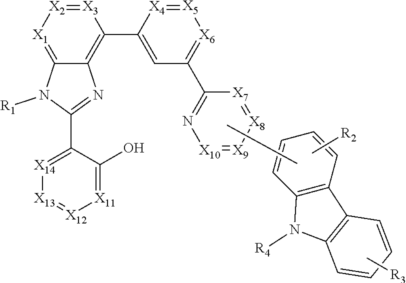

9. An organic photoelectronic device comprising the platinum complex of claim 1, wherein the photoelectronic device is an organic light-emitting diode, an organic thin film transistor, an organic photovoltaic device, a luminescent electrochemical cell, or a chemical sensor.

10. An organic light-emitting diode, comprising a cathode, an anode, and an organic layer, wherein the organic layer comprises one or more of a hole injection layer, a hole transport layer, a light-emitting layer, a hole blocking layer, an electron injection layer, or an electron transport layer, and at least one of the hole injection layer, the hole transport layer, the hole blocking layer, the electron injection layer, the light-emitting layer, or the electron transport layer comprises the platinum complex of claim 1.

11. The organic light-emitting diode of claim 10, wherein the platinum complex is used as a luminescent material in the light-emitting layer or as an electron transport material in the electron transport layer.

12. An organic light-emitting diode, comprising a cathode, an anode, and an organic layer, wherein the organic layer comprises one or more of a hole injection layer, a hole transport layer, a light-emitting layer, a hole blocking layer, an electron injection layer, or an electron transport layer, and at least one of the hole injection layer, the hole transport layer, the hole blocking layer, the electron injection layer, the light-emitting layer, or the electron transport layer comprises the platinum complex of claim 2.

13. An organic light-emitting diode, comprising a cathode, an anode, and an organic layer, wherein the organic layer comprises one or more of a hole injection layer, a hole transport layer, a light-emitting layer, a hole blocking layer, an electron injection layer, or an electron transport layer, and at least one of the hole injection layer, the hole transport layer, the hole blocking layer, the electron injection layer, the light-emitting layer, or the electron transport layer comprises the platinum complex of claim 3.

14. An organic light-emitting diode, comprising a cathode, an anode, and an organic layer, wherein the organic layer comprises one or more of a hole injection layer, a hole transport layer, a light-emitting layer, a hole blocking layer, an electron injection layer, or an electron transport layer, and at least one of the hole injection layer, the hole transport layer, the hole blocking layer, the electron injection layer, the light-emitting layer, or the electron transport layer comprises the platinum complex of claim 4.

15. An organic light-emitting diode, comprising a cathode, an anode, and an organic layer, wherein the organic layer comprises one or more of a hole injection layer, a hole transport layer, a light-emitting layer, a hole blocking layer, an electron injection layer, or an electron transport layer, and at least one of the hole injection layer, the hole transport layer, the hole blocking layer, the electron injection layer, the light-emitting layer, or the electron transport layer comprises the platinum complex of claim 5.

16. An organic light-emitting diode, comprising a cathode, an anode, and an organic layer, wherein the organic layer comprises one or more of a hole injection layer, a hole transport layer, a light-emitting layer, a hole blocking layer, an electron injection layer, or an electron transport layer, and at least one of the hole injection layer, the hole transport layer, the hole blocking layer, the electron injection layer, the light-emitting layer, or the electron transport layer comprises the platinum complex of claim 6.

17. An organic light-emitting diode, comprising a cathode, an anode, and an organic layer, wherein the organic layer comprises one or more of a hole injection layer, a hole transport layer, a light-emitting layer, a hole blocking layer, an electron injection layer, or an electron transport layer, and at least one of the hole injection layer, the hole transport layer, the hole blocking layer, the electron injection layer, the light-emitting layer, or the electron transport layer comprises the platinum complex of claim 7.
